(19) United States
(12) Patent Application Publication
(10) Pub. No.: US 2015/0250824 A1
(43) Pub. Date: Sep. 10, 2015

(54) METHODS AND COMPOSITIONS FOR EXPANSION OF STEM CELLS AND OTHER CELLS

(71) Applicant: The Research Foundation For The State University Of New York, Albany, NY (US)

(72) Inventor: Yupo MA, East Setauket, NY (US)

(21) Appl. No.: 14/638,920

(22) Filed: Mar. 4, 2015

Related U.S. Application Data
(60) Provisional application No. 61/949,769, filed on Mar. 7, 2014.

(51) Int. Cl. A61K 35/28; A61K 31/506; A61K 31/4375; A61K 31/135; C12N 5/071; A61K 31/437; A61K 31/444; A61K 31/4439; A61K 31/551; C12N 5/0735; A61K 31/19; A61K 31/352

(52) U.S. Cl. CPC A61K 35/28; A61K 31/19; A61K 31/506; A61K 31/4375; A61K 31/135; A61K 31/352; A61K 31/437; A61K 31/444; A61K 31/4439; A61K 31/551; C12N 5/0606; C12N 5/0676; A61K 2035/124

(57) ABSTRACT

Presented herein are methods of generating a multipotent or immature cell from a mature somatic cell, involving contacting a mature somatic cell with one or more small molecule compounds selected from: a histone deacetylase (HDAC) inhibitor; a glycogen synthase kinase 3 (GSK-3) inhibitor; one or more transforming growth factor-beta receptor (TGF-βR) inhibitors; one or more lysine-specific demethylase 1 (LSD1) inhibitors; a cAMP agonist; a histone lysine methyltransferase (EZH2) inhibitor; and a histone methyltransferase (HMTase) G9a inhibitor; valproic acid. Also provided are methods of generating a multipotent or immature cell from a somatic cell, by driving expression of OCT4, or an OCT4 functional homolog or derivative, under the control of a high expressing promoter. Presented herein are also methods of stem cell expansion, stem cell regeneration and differentiation, which comprise contacting stem cells with one or more small chemical compounds.

### CROSS REFERENCE TO RELATED APPLICATIONS

This application claims priority from U.S. Provisional Application No. 61/949,769 filed Mar. 7, 2014, the entire contents of which are incorporated herein by reference.

### INCORPORATION BY REFERENCE OF SEQUENCE LISTING

The Sequence Listing in the ASCII text file, named as 30665_SequenceListing.txt of 13 KB, created on Mar. 4, 2015, and submitted to the United States Patent and Trademark Office via EFS-Web, is incorporated herein by reference.

### BACKGROUND OF THE DISCLOSURE

Stem cells are undifferentiated cells that have extensive proliferation potential, can differentiate into several cell lineages, and repopulate tissues upon transplantation. Stem cells can give rise to more progenitor cells having the ability to generate a large number of mother cells that can in turn give rise to differentiated or differentiable daughter cells.

Hematopoetic stem cells (HSCs) are stem cells that are capable of differentiating into three cell lineages including myeloerythroid (red blood cells, granulocytes, monocytes), megakaryocyte (platelets) and lymphoid (T-cells, B-cells, and natural killer) cells (Robb et al. Oncogene, 2007, 6715-6723). These HSCs are used in clinical transplantation protocols to treat a variety of diseases including malignant and non-malignant disorders. Expansion of HSCs has important clinical applications since the relative inability to expand hematopoetic stem cells ex vivo imposes major limitations on the current use of HSC transplantation. There is a shortage of HSCs used for patient treatments related to bone marrow transplantation or genetic disorders (Heemskerk et al. 2005, Bone Marrow Transplantation 35, 645-652) For allogenic bone marrow transplantation, only one third of all patients who would potentially benefit from an HSC transplant will find a suitable human leukocyte antigen (HLA)-matched related donor.

Hemangioblasts are multipotent stem cells that can differentiate into both hematopoietic and blood vessel endothelial cells. Hemangioblasts express FLK1 and are first found from embryonic cultures and can be manipulated by cytokines to differentiate along either the hematopoietic or endothelial route. Hemangioblasts are in the tissue of post-natal individuals, such as in newborn infants and adults. The immunophenotypic feature of this population is FLK+, CD34−CD31−. Hemangioblasts have multiple potential applications in treatment of a variety of diseases including cardiovascular diseases, diabetes and stroke. Hemangioblasts could be used to generate hematopoietic stem/progenitor cells for treating hematologic malignancies and other disorders. Hemangioblasts could also be a potentially unlimited source of platelets and red blood cells for transfusion (Jaffrdo et al. 2005, Experimental Hematology, 33, 1029-1040)

Type 1 diabetes affects about 4-5% of the world's population and can be reversed by pancreatic islet beta cell transplantation. Human β-cells, generated in ample quantities, are a prerequisite in order to realize a wider application of β-cell replacement therapy for diabetes. However, acute shortage of organ donors, lifelong immunosuppression and chronic graft rejection currently limit greater use of this potentially curative therapy. Attempts at culturing adult human islet cells result in a loss of beta-cell functions. In addition, these cultures undergo senescence following 15 population doublings (Rutti et al 2012, PLoS One; 7(4): e3580). Finding a source of a sufficient number of functioning islet beta cells is urgently needed. A source of autologous beta cells would solve issues related to supply and graft rejection.

Somatic cells have recently been reported to reprogram into pluripotent cells, termed induced pluripotent stem (iPS) cells, using a combination of defined transcription factors (Takahashi et al, Cell 126:663-676, 2006; Okita et al, Nature 448:313-317, 2007; Takahashi eta al, Cell 131:861-872, 2007; Lewitzky et al., Curr Opin Biotechnol. 2007; 18:467-473). The reprogramming of somatic cells to iPS cells is a new area of significant potential. These cells have great therapeutic potential because they can be tailored specifically to a patient or disease. In principle, an individual suffering from a genetic, degenerative, or malignant disorder could submit a biopsy for reprogramming to an iPS cell. Following reprogramming, a prescribed course of iPS cell differentiation to a specific tissue type could be initiated that would allow one to cure a given disorder. Proof of principle experiments have been done in mouse models. For example, mice displaying a phenotype similar to human sickle cell anemia were cured of the disease through somatic cell reprogramming and directed differentiation into blood cell progenitor populations (Hanna et al, Science. 2007; 318:1920-1923). This is a clear demonstration of potential therapeutic uses for iPS cells.

iPS cells, like embryonic stem (ES) cells, have numerous challenges, including genetic instability and cancer risk. For example, activation of exogenously-introduced iPS-inducing genes may lead to the malignant transformation of iPSs (for example, when oncogenic transcription factors, such as c-Myc, are used). Transdifferentiation, a process of reprogramming a cell directly from one mature cell type to another cell type, has also been reported (Jopling et al, Nature Reviews Molecular Cell Biology, 2011, 12, 79-89). In these methods, mature cells, and not pluripotent stem cells, are produced, which reduces the risks of cancer and genetic instability of the induced cells. Unfortunately, the mature cells derived from direct reprogramming are likely insufficient for cellular therapy due to their limited capacity to self-renew and regenerate. Because of this limitation, direct reprogramming of somatic cells into multipotent or lineage-restricted stem cells is preferred because such cells could have adequate capacity of self-renewal and differential potential, yet have reduced tumorigenic potential.

Several published research accounts have reported the direct reprogramming of somatic skin cells to neural stem cells (NSCs) using a lentiviral vector expressing SOX2 or a combination of defined transcriptional factors(Kim et al, Proc Natl Acad Sci USA, 2011, 18:7838-43; Ring et al, Cell Stem Cell 11, 100-109, Jul. 6, 2012). However, the described processes require co-culturing the somatic cells with feeder cells, which carries additional risks. Finally, the efficiency of direct reprogramming of such cells is very low, resulting in insufficient numbers for clinical use. The safety issues and low efficiency of direct reprogramming are also barriers for clinical applications of these cells.

Only a study in mice shows that iPS cells can be generated from mouse somatic cells using a combination of six small-molecule compounds alone: valproic acid; CHIR99021, a glycogen synthase kinase 3 inhibitor; 616452, a transforming growth factor-beta receptor inhibitor II; FSK, a cAMP agonist; and DZNep, 3-deazaneplanocin A (Hou et al., Science, 2013, 341: 651-654). However, it remains to be determined if these compounds alone are able to function in a similar fashion in humans as they have not been reported in the generation of pluripotent stem cells in humans. Accumulated documents show that significant differences exist in the transcriptional networks and signaling pathways that control mouse and human pluripotent stem cell self-renewal and lineage development (Schnerch el al, Stem Cells 2010; 28(3):419-30).

There are clear advantages in the use of small molecules for reprogramming, but to date have not been shown that multipotent stem cells or tissue specific stem cells could also be generated by small chemical molecules alone.

There are numerous small molecules in the combination of overexpression of transcription factors involving an iPSC reprogramming process. It is likely that chemically defined reprogramming for iPS cells and multipotent stem cells involves different combination of small molecules. A method to screen these compounds and define a set of small molecules would be invaluable for providing the best protocol to generate human multipotent stem cells or immature cells for therapy.

Severe aplastic anemia: A blood disorder where the bone marrow produces insufficient new blood cells and bone space lacks hematopoietic stem cells. There are various treatments for aplastic anemia including blood transfusion, bone marrow-stimulating agents, and bone marrow transplant.

Leukopenia: A blood disorder which involves a decrease in the number of white blood cells (leukocytes) found in the blood, which places individuals at increased risk of infection. Neutropenia can be caused by impaired production of neutrophils in the bone marrow due to inadequate marrow stem, or by accelerated destruction of neutrophils.

Neutropenia and its complications are among the most common and serious adverse effects of chemotherapy, radiation therapy, and bone marrow transplantation.

Acute radiation syndrome (ARS) (also known as radiation poisoning, radiation sickness or radiation toxicity): radiation-induced neutropenia associated with ARS due to exposed to high level radiation, such as a nuclear incident.

Accumulated documents have shown that bone marrow derived progenitor cells as a tool for regeneration medicine are indeed promising in the treatment of diseases such as heart diseases. The hematopoietic stem/progenitor cells, endothelial progenitor cell (EPC) and bone marrow derived mesenchymal stem cells (MSCs) have all been shown to improve the perfusion and new vascular development within the infarct. Peripheral blood progenitor cells (PBSC) have a long history of treating cancer. Recently PBSCs have been extended to treat other diseasesor cell injury. Some PBSC trials established reasonable improvement in autoimmune diseases

Megakaryocytes are one of few cell types that undergo endomitosis, a form of cell cycle that skips the late stages of mitosis to become polyploid. Human megakaryocytes commonly reach ploidy states of 16N and can sometimes achieve states as high as 64N or greater. The mechanism of polyploidization is still not well understood, however, polyploidy is required for functional human megakaryocyte maturation. Once active, the megakaryocytes are responsible for the production of platelets that have well-characterized rolls in hemostasis, thrombosis, vascular integrity, development of the lymphatic system and the innate immune response

Thrombocytopenia affects 20-30% (25,000-30,000 per year) of infants admitted to the neonatal intensive care unit. Approximately 9% of those infants are severe and experience clinically significant bleeding (usually intracranial). Platelet transfusions are the only therapeutic option for thrombocytopenic neonates. Recent studies have shown that megakaryocytes of neonates are smaller and have lower ploidy than those of adults. Megakaryocytes achieve adult size at approximately 1 year of age. Small megakaryocytes usually produce fewer platelets than large megakaryocytes. Therefore, an inability to increase megakaryocyte size and ploidy in response to increased platelet consumption might underlie the predisposition of sick neonates to thrombocytopenia.

Human umbilical cord blood (CB) is an important stem cell source for patients who lack other suitable donors. However, slower platelet engraftment is a major drawback of CB transplantation. Platelet engraftment takes an average of approximately 70 days for CB recipients, versus 20 days for mobilized peripheral blood cells derived from adult donors.

Megakaryocyte stem/progenitor cells from neonatal and older donors in terms of the differences in their ability to produce large megakaryocytes after transplantation may contribute to the delayed platelet engraftment after CB transplantation. Identification of a megakaryocyte maturation inducer or co-transfusion of large numbers of ex vivo generated human megakaryocyte (Mk)-committed cells with high maturation potential, could provide an alternative method to shorten period of thrombocytopenia. Thrombopoietin (TPO) and derivatives have been used in the treatment of thrombocytopenia in adult patients. However, the observations of no excessive bleeding in Tpo−/− mice and the limited positive therapeutic efficacy of TPO have attracted more and more attention to TPO-independent megakaryocytopoiesis (Zheng et al, Critical Reviews in Oncology/Hematology, 65 (2008), 212-222). Studies in models using the nonhuman primate or canine have also demonstrated that standard post-transplant admiration of TPO does not accelerate platelet reconstitution following AuBMT or alloBMT, respectively, in myeloablated hosts. TPO stimulates the megakaryocyte formation in vivo, but it does not shorten its maturation time.

Differentiation therapy with small molecules is considered a powerful approach to target specific types of leukemia. One of the best examples is the use of All-trans-Retinoic acid (ATRA) in treating Acute Promyelocytic Leukemia (APL), which has significantly improved clinical outcome. In acute megakaryoblastic leukemia, polyploidization and differentiation are blocked. As a result, low ploidy megakaryoblasts become a predominant form in the leukemic blasts. Clinical studies with polyploidy inducers, such as Aurora kinase A inhibitors, have been used for a wide variety of hematologic malignancies including acute megakaryocytic leukemia, chronic myeloproliferative disorder and myelodysplastic syndromes. However, treatment with these inhibitors is often associated with severe side effects. Development of novel forms of differentiation therapy is clinically important.

Thus, there remain numerous barriers to be solved before these promising therapies are ready for use in human subjects.

### BRIEF SUMMARY OF THE DISCLOSURE

Presented herein are methods of generating a pluripotent or immature cell from a somatic cell, by driving expression of OCT4, or an OCT4 functional homolog or derivative, under the control of a high expressing promoter such as spleen focus forming virus (SFFV) and human elongation factor 1α (EF) promoter, in the somatic cell. The somatic cell can be a mature somatic cell. The somatic cell can be selected from an umbilical cord blood cell, an amniotic fluid cell, a bone marrow cell, a blood cell, a myocardial cell, a dermal or epidermal cell, a pancreatic cell, endothelial cell, liver cell or a fibroblast. The somatic cell can be a CD34+ cell or a pancreatic islet cell.

The generated cell can be a pluripotent cell such as a neural stem cell (NSC), bone stem cell, bone marrow stem cell, lung stem cell, kidney stem cell, endothelial stem cell, myocardial stem cell, muscle stem cell, mesenchymal stem cell, hepatic stem cell, pancreatic stem cell, dermal stem cell, epidermal stem cell, hemangioblast, or hematopoietic stem cell. The generated cell can be an immature cell such as an immature pancreatic beta cell, particularly an immature pancreatic beta cell that can produce insulin, and/or that can differentiate into or give rise to an insulin-producing cell.

To generate a multipotent or immature cell, the somatic cell is transduced with an integrative or episomal vector with a nucleic acid sequence encoding OCT4 or an OCT4 functional homolog or derivative. The inventors have determined that driving OCT4 expression via a highly expressing and preferably constitutive promoter is sufficient to induce reprogramming of a somatic cell into the desired multipotent or immature cell. In some embodiments, the vector is a lentiviral vector. In other embodiments, the vector is an episomal or non-integrative vector, such as an episome derived from a pCEP plasmid.

The methods disclosed herein provide for reprogramming of somatic cells in vitro or ex vivo in the absence of feeder cells. The methods can involve, for example, reprogramming a CD34+ cell, such as a cord blood CD34+ cell, in neural stem cell medium to generate a neural stem cell, or reprogramming a CD34+ cell, such as a cord blood CD34+ cell, in embryonic stem cell medium to generate a hemangioblast/hematopoietic stem cell, or reprogramming a mature pancreatic islet cell in embryonic stem cell medium to generate an immature pancreatic beta cell.

In some embodiments, a CD34+ cell is transduced with an integrative vector in media that includes a glycogen synthase kinase 3 (GSK-3) inhibitor, such as the GSK-3 inhibitor CHIR99021. In other embodiments, the transduced CD34+ cell is cultured, after transduction, in medium with 3-deazaneplanocin. The transduced somatic cell can be cultured in vitro or ex vivo for at least five days to generate a multipotent or immature cell.

In another embodiment, the reprogrammed multipotent or immature cells by the disclosure method bear a plasticity property. For example, reprogramming a CD34+ cell, such as a cord blood CD34+ cell, in embryonic stem cell (ESC) medium or mesenchymal stem cell medium (MSC) to generate FLK1+ cells and these are capable of conversion to other types of stem cells, such as neural stem cells.

This disclosure further provides methods of generating a multipotent or immature cell from a human somatic cell, involving contacting a human somatic cell with one or more compounds selected from: an HDAC inhibitor; a transforming growth factor-beta receptor (TGF-βR) inhibitor II; an activin receptor-like kinase 4 (ALK4), ALK5 or ALK7 inhibitor; a glycogen synthase kinase 3 (GSK3) inhibitor; a lysine methyltransferase EZH2 inhibitor; a histone-lysine methyltransferase (HMTase) inhibitor; an inhibitor of the histone lysine demethylase LSD1; and a histone methyltransferase G9a/GLP inhibitor.

In some embodiments, the HDAC inhibitor is valproic acid. In some embodiments, the GSK-3 inhibitor is CHIR99021 or a functional derivative thereof. In some embodiments, the TGF-βR inhibitor is 616452 or a functional derivative thereof. In some embodiments, the inhibitor of LSD1 is tranylcypromine hydrochloride or a functional derivative thereof. In some embodiments, the cAMP agonist is forskolin or a functional derivative thereof. In some embodiments, the HMTase G9a inhibitor is BIX 01294 or a functional derivative thereof. In some embodiments, the EZH2 inhibitor is 3-deazaneplanocin A or a functional derivative thereof.

In a specific embodiment, hematopoietic stem cell or progenitor cells can be expanded in vitro or ex vivo with a combination of a TGF-βR inhibitor, such as 616452 or a functional derivative thereof, and an EZH2 inhibitor, such as 3-deazaneplanocin A or a functional derivative thereof. In another specific embodiment, the human hematopoietic stem cells or progenitor cells can be expanded in vitro or ex vivo with a combination of: a GSK-3 inhibitor, such as CHIR99021 or a functional derivative thereof; a TGF-βR inhibitor, such as 616452 or a functional derivative thereof; an inhibitor of the histone lysine demethylase LSD1, such as tranylcypromine hydrochloride; a cAMP agonist, such as forskolin or a functional derivative thereof, and an EZH2 inhibitor, such as 3-deazaneplanocin A or a functional derivative thereof. In another embodiment, the human somatic cell is a CD34+ cell cultured in vitro or ex vivo with a combination of an EZH2 inhibitor, such as 3-deazaneplanocin A or a functional derivative thereof, and an HMTase G9a inhibitor, such as BIX 01294 or a functional derivative thereof.

The inventors have demonstrated that TGF-beta receptor 1 inhibitor, 616452 can enhance megakaryocyte differentiation and drastically shorten megakaryocyte maturation. In some embodiments, the disclosure provides a method of promoting hematopoietic recovery, particularly platelets in a subject, comprising administering a TGF-beta receptor 1 inhibitor, 616452 or its functional derivative thereof.

The disclosed methods promote differentiation of fetal/neonatal megakaryocyte cells. In some embodiments, the disclosed methods may promote differentiation of fetal/neonatal megakaryocyte cells into mature platelet-producing cells.

The disclosure includes methods for reducing abnormal or malignant megakaryocytes in bone marrow or blood. In some embodiments, malignant megakaryocytes are present in patients with myelodysplastic syndromes, chronic myeloproliferative disorders and acute myeloid leukemia.

This disclosure further provides a multipotent or immature cell produced by any of the disclosed methods. The multipotent or immature cell can be, for example, an induced neural stem cell (iNSC), a hemangioblast/hematopoietic stem cell, a megakaryocyte, or an immature pancreatic beta cell.

This disclosure further provides methods of treating conditions associated with a cellular deficiency in a subject, comprising administering a multipotent or immature cell generated according to the disclosed methods to a subject in need thereof. In some embodiments, the condition is myocardial infarction, congestive heart failure, stroke, ischemia, peripheral vascular disease, alcoholic liver disease, cirrhosis, a neurodegenerative disease, diabetes, cancer, arthritis, a wound, immunodeficiency, leukemia, anemia, or a genetic disorder. In preferred embodiments, the multipotent or immature cell is autologous to the subject being treated.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1B. The timeline of formation of the hemangioblast-like colonies from CB CD34⁺ cells. FIG. 1A, timeline. FIG. 1B, clones of 1-4 cells appear after 24 hours which begin to proliferate and expand. After 5 days, the colonies begin to demonstrate a high proliferative potential and reach confluence by around day 10. Cells are passaged and can be expanded thus far for 1-2 months while maintaining their characteristics.

FIGS. 2A-2D. Morphology of colonies and stain with FLK antibody. Two colonies are manually picked up (A and B). The colonies are dissociated (C) into single cells and they are FLK1 positive in more than 95% of cells (D).

FIG. 3. Time line after transduction of episome into CB CD34+ cells. Cells were passaged on day 8 and grew at a doubling rate of approximately 16 hours.

FIG. 4. Hemangioblast-like cells can differentiate into endothelial cells as identified by VE-cadherin and CD31 immunofluorescence.

FIG. 5. Hemangioblast-like cells can differentiate into endothelial/macrophage cells as identified by Ac-LDL (acetylated low density lipoprotein) uptake.

FIGS. 6A-6C. Reprogramming of CB CD34+ cell to iNSCs by lentiviral OCT4 overexpression. A. The diagram of the reprogramming procedure. B. Adherent appearance in OCT4 and control group. C. in vitro long-term expansion of CB-iNSCs.

FIGS. 7A-7B. Characterization of CB-iNSCs. A. Relative expression of neural stem cells or hematopoietic stem cell genes of CB-iNSCs to CB CD34+ cells as quantitated by real-time PCR analysis. B. Neurosphere formation of CB-iNSCs. Scale bars=50

FIGS. 8A-8E. In vitro differentiation of CB-iNSCs. A. Differentiated CB-iNSCs show neuron-like morphology with long neurites as compared to undifferentiated cells. B. Multilineage differentiation of CB-iNSCs as shown by staining of neuron (Tuj1), astrocyte (GFAP) or oligodentrocyte (CNPase) marker immunostainings. C. Synapse-like structure in differentiated CB-iNSCs. D. PCR analysis of genes related to neurotransmitter and multilineage neural cells, i: Undifferentiated CB-iNSC 1, ii: Differentiated CB-iNSC 1, iii: Differentiated CB-iNSC 3. E. Regional pattern of CB-iNSCs.

FIGS. 9A-9D. In vivo transplantation of CB-iNSCs. A-B. The GFP labeled CB-iNSCs (green) could be seen in the injection region (right striatum, marked ellipse area) at one month and three months post transplantation. C. Cell densities of migrated CB-iNSCs in contralateral (left) hemisphere at one month and three months post transplantation (P<0.01). D. Neurons differentiated from CB-iNSCs three months post transplantation.

FIG. 10. Generation of eiNSCs with an episomal vector. Adherent cell appearance after episomal transfection and in vitro culture of eiNSCs.

FIG. 11. The timeline of formation of beta cell-like colonies. About 20 small reprogrammed colonies were observed in each 5×10³ beta cell seeded well day 17 to 19 after viral infection.

FIGS. 12A-12B. Timeline for the colony derivation (top) and characteristic colonies (A-D). (A) Primary, fresh islets were dissociated with accutase and plated on a collagen I coated plate. Lentiviral vector expressing GFP under the SFFV promoter was used as a control for determination of infection efficacy. (B) By Day 7, “transformed” colonies were visible in the islet cells transduced with either SFFV-OCT4 or EF1-OCT4 lentiviruses. (C) Immunofluorescence analysis of colonies. Surface marker for Flk-1 was not observed in isolated islet cells, but after direct reprogramming with lentiviral vector expressing Oct4, all colonies became strongly positive for Flk-1. (D) Reprogrammed colonies stained with CXCR4 antibody. Immunofluorescence studies showed that endoderm marker, CXCR4 was also observed in the Flk-1 positive colonies.

FIG. 13. Comparisons of immunophenotypic effects by different compounds on hematopoietic stem/progenitor cell expansion. CD34+ cells isolated from peripheral blood of G-CSF-mobilized donors were cultured for 11 days under minimal cytokines and different compounds, C (at 10 μM), 6 (at 10 μM), F (at 10 μm) and Z (at 100 nM). Chemical C did not have much of effect on either the CD34+CD38− or CD34+CD38− population. Chemical F induced a significant expansion of CD34+CD38+ while compound 6 and Z appeared to be the most effects on the CD34+CD38− population. The CD34+CD38− population is associated with long-term HSC repopulation.

FIG. 14. A combination of compounds 6 and Z on CD34+ cells better expands and maintains CD34+CD38− populations. Immunophenotypic analysis of precursor cells in cultured CD34+ bone marrow (top panels) and human cord blood (bottom panels).

FIG. 15. Compound Z or Bix enhances retention and expansion of CD34+CD38-population. Z-1×, Z at 100 nM.

FIG. 16. Compound Z or T promotes hematopoietic precursor immunophenotype and expands CD34+CD38− population. Z-1×, Z at 100 nM; Z-0.5, Z at 50 nM.

FIG. 17. Dose-dependent activation of the SALL4 promoter by TGF βR1 kinase inhibitor II, 616452. 0.1 μg of the SALL4-Luc promoter construct was co-transfected with 0.05 μg of Renilla plasmid in HEK-293 cells and the resulting transfected cells were exposed to different concentrations of 616452. Y axis: relative luciferase.

FIGS. 18A-18D. Induction of megakaryocytic differentiation and maturation in bone marrow CD34+. (A) The left shows the control BM CD34+ cells after 10 days culture while the right is cells induced with chemical (10 μM). (D), a magnified picture of induced cells after 10 days culture. (B), a live cell stain using CD41. (C), flow cytometry results comparing the control and chemical using CD41 as the y-axis and CD34 on the x-axis after 5 days of induction with the small chemical. Between different experiments (N>3), the percentage of CD41 increased 50-100% after 5 days of culture. The presence of the chemical yields a higher percentage of CD34+.

FIGS. 19A-19C. Induction of megakaryocytic differentiation and maturation in CD34+ cells isolated from human cord blood. Chemical induction of hUCB CD34+ derived from healthy donors. (A), the left side is the control while the right side is chemically induced (10 μM). (B), a Giemsa-Wright Stain after 8 days culture depicting megakaryocytes with the multi-nucleated and lobular nature of the nuclei. Morphology also illustrates a granular nature of the cytoplasm, typical of megakaryocytes. (C), the flow results comparing the control and chemical using CD41 as the y-axis and CD34 on the x-axis after 5 days of induction with 616452.

FIGS. 20A-20B. Ploidy analysis of the MKs derived from CB CD34+ cells. (A), cells were induced with 616452 at 10 μM for 8 days (left side) or 12 days (right side). Analysis was done on the respective days using PI staining and Flow Cytometer. (B), dose dependent analysis run on CB CD34+ cells where 616452 was cultured at various concentration and analyzed for ploidy as of day. All the cells were grown with TPO in addition to the chemical.

FIGS. 21A-21H. Chemical 616452 effect of maturation on CD41 expressing megakaryocyte stem/progenitor cells. (A) and (C), control CD41+ cells induced with only DMSO; (B) and (D), CD41+ cells induced with 616452. (E) and (F) show the cluster that formed by day 8. (G) and (H), clusters labeled with CD41 to depict the morphology and the constituents of the clusters.

FIG. 22. Chemical 616452 effect of polyploidization on CD41 expressing megakaryocyte stem/progenitor cells. CB CD34+ cells were cultured with TPO, SCF, IL-3 for 7 days prior for CD41 selection by flow cytometry. Control (DMSO induced) and 616452 induced cells were grown in StemSpan containing only TPO for 8 days prior to cell cycle analysis. In control cells we observed only 6% of the cells increased their ploidy numbers with no cells reaching a ploidy of 16N or greater. Meanwhile with 616452-induced cells, we observed at least 54% of the cells had a nuclei of 4N or greater with a 4.5 fold increase in the number of 4N, 30 fold increase in 8N and the generation of cells with ploidy reaching as high as 64N within 8 days of culture.

FIGS. 23A-23B. Synergistic effect of three TGF-beta inhibitors on CB− megakaryocyte maturation. (A) CB CD34+ cells were induced with each chemical for 4 days before cells were analyzed by flow cytometry with markers, CD41 and CD34. On the top row, the order from left to right is 616454 (4), SB431452 (5), 616452 (6). On the bottom row is control cells induced with DMSO on the left and all 3 inhibitors combined on the right. (B) is the ploidy analysis of the cells after 4 and 8 days of induction with the chemical. Visually, there were no significant difference between the other inhibitors and the control while the combined inhibitors had a drastic effect as of day 4. Analysis on day 8 may be underestimated due to the formation of the clusters. Controls are cells induced with DMSO.

FIG. 24. Gene expression profile of CB CD34+ cells induced by 616452 for 2 and 4 days compared to control. There are noticeably more hematopoietic regulatory genes expressed and at higher levels on 4 days of induction compared to that of day 2. There are 18 genes total up-regulated on day 2, and by day 4, there were 38. On the right is a heat map illustrating the values of expression in terms of fold increase over controls. Green is indicative of up-regulation while red is down-regulation.

FIG. 25. Robust enhancement of platelet recovery by 616452. Fifteen 8-week old mice were given IP injections of 5-FU (250 mg/kg) on day 0. Complete blood counts (CBCs) were done on day 2, 4, 6, 9, and 12. 616452 (10 mg/kg) was introduced via IP injections on day 5, 7 and 9. The above data is a representative sample of the overall experiment. Data values from day 4 are not included.

FIG. 26. FLK1 expression of chemical treated AF cells. On day 7, AF cells became positive for FLK1 expression in both 6-chemical and 8-chemical treated groups, while no positive cells were found in a control group.

### DETAILED DESCRIPTION OF THE DISCLOSURE

This disclosure provides methods of generating a multipotent or immature cell from a human somatic cell, involving contacting a human somatic cell with one or more compounds selected from: a histone deacetylase (HDAC) inhibitor, a transforming growth factor-beta receptor (TGF-βR) inhibitor, a glycogen synthase kinase 3 (GSK) inhibitor; an activin receptor-like kinase (ALK) inhibitor (e.g., an ALK4, ALK5 and/or ALK7 inhibitor), a histone lysine methyltransferase/Enhancer of zeste homolog 2 (EZH2) inhibitor, and a histone methyltransferase (HMTase) G9a inhibitor. The methods can involve culturing the human somatic cell in vitro or ex vivo with one or more, two or more, three or more, or four or more of these compounds or their derivatives, or administering the one or more compounds to a subject to generate a multipotent or immature cell from a human somatic cell in vivo.

Based on a hypothesis-driven approach and screenings of small molecules that target cellular mechanisms that are known to influence stem cell properties, the inventors identified subsets of chemical compounds that can reprogram or dedifferentiate somatic cells to multipotent or immature cells.

Also presented herein are methods of generating a multipotent or immature cell from a somatic cell, by driving expression of OCT4, or an OCT4 functional homolog or derivative, under the control of a high expressing promoter such as spleen focus forming virus (SFFV) or elongation factor-1 (EF1) promoter. The process of generating multipotent or immature cells “skips” the embryonic state.

The generated cell can be a multipotent stem cell or immature cell such as a neural stem cell (NSC), bone stem cell, bone marrow stem cell, lung stem cell, kidney stem cell, endothelial stem cell, myocardial stem cell, muscle stem cell, mesenchymal stem cell, hepatic stem cell, pancreatic stem cell, dermal stem cell, epidermal stem cell, hemangioblast, or hematopoietic stem cell. The generated cell can be an immature cell such as an immature pancreatic beta cell, particularly an immature pancreatic beta cell that can produce insulin, and/or that can differentiate into or give rise to an insulin-producing cell.

The ability to dedifferentiate mature lineage restricted cells into more primitive versions of the same cell lineage capitalizes on cell renewal properties while minimizing the risk of malignancy. The generated cells can be rapidly propagated with a doubling time of 14 hours in vitro without senescing, or exhibiting signs of genetics instability, or loss of potential after 30 passages.

In addition, the inventors have biologically engineered hemangioblast-like cells to be large-scale endothelial cell factories to provide substantial numbers of pure endothelial cells for use in therapy. Since endothelial cells are derived from hemangioblasts, a close developmental relationship exists between these cell types. Endothelial cells' pre-existing epigenetic program allows for dedifferentiation to generate hemangioblast-like cells.

In support of this, the inventors show that: (1) endothelial cells are far more responsive to OCT4 than that of previously described fibroblasts in generation of primitive stem cells, generating ˜100% of Fetal Liver Kinase 1 (FLK1)+CD31-CD34− cells after reprogramming FLK+CD31-CD34− cells represent a rare population in the human adult body. The inventors' invented technology is able to generate unlimited, large-scale numbers of FLK+CD31-CD34-cells because these dedifferentiated cells can be robustly, indefinitely propagated in vitro. The dedifferentiated FLK+ hemangioblast cells are able to cross-lineage restriction boundary and give rise to cell types of other lineages, such as neural stem/progenitor cells. In a similar strategy, these could give rise to precursor cells for insulin-secreting beta cells, endothelial cells, bone marrow cells, fat cells, liver cells, kidney cells and lung cells.

(2) Dedifferentiated hemangioblast-like cells cultured in endothelial medium are capable of robust differentiation to abundant endothelial cells with near 100% conversion efficiency in only 5 days. As a comparison, the inventors observe that only approximately 1% iPS (induced pluripotent stem cell) intermediates can be derived from skin in 28 days. This epigenetic/developmental memory predisposes dedifferentiation-derived stem cells to differentiate more readily into their mature counterparts. This skewed differentiation potential is advantageous for cell replacement therapy.

(3) Endothelial cells derived from dedifferentiated hemangioblast-like cells bear functional properties including in vitro functional AC-LDL (Acetylated-low density lipoprotein) uptake and tubular formation in the matrigel.

(4) Dedifferentiated hemangioblast-like cells cultured in hematopoietic cell medium are capable of differentiation to hematopoietic cells expressing CD45.

There is significant therapeutic potential for the transplantation of gene-corrected, patient-specific iPS-derived hematopoietic stem cells (HSCs) in patients with various hematological disorders and hematologic malignancies. However, there have been numerous attempts to demonstrate the marrow repopulating ability of pluripotent stem cells, PSCs (hESC or iPS) derived hematopoietic progenitor cells in immunocompromised mice, and none of these studies has thus far demonstrated significant levels of multilineage marrow repopulation in serial transplants. PSC derived hematopoietic stem cells (PSC-HPCs) engraft poorly, even after intra-bone marrow administration.

Current methodologies, focusing on stimulating hemogenesis from primitive hemangioblasts which appear earliest during embryoid body (EB) differentiation, give rise only to primitive yolk sac-like hematopoietic cells (e.g. embryonic erythroblasts) rather than to the definitive hematopoietic cells derived from relative mature hemangioblasts. One of invention features the ability to generate and robustly expand hemangioblast-like cells. The hemangioblast-like cells are likely more mature than primitive hemangioblasts and may be more efficiently differentiate to different lineages of hematopoietic cells.

The term “stem cell” as used herein refers to an immature cell that is capable of differentiating into a number of final, differentiated cell types. A stem cell may divide symmetrically, to form two daughter stem cells, or asymmetrically, to form a daughter stem cell and a somatic cell. Characteristics of stem cells include loss of contact inhibition, anchorage independent growth, de novo expression of alkaline phosphatase and/or activation of Oct4. Oct4, a member of the Pou domain, class 5, transcription factors (Pou 5fl) (Genbank Accession No. 568053) is one of the mammalian POU transcription factors expressed by early embryo cells and germ cells, and is a marker for stem cells in mammals.

Stem cells may be totipotent, pluripotent, or unipotent cells. Totipotent stem cells typically have the capacity to develop into any cell type and are usually embryonic in origin. Pluripotent cells are typically capable of differentiating into several different, final differentiated cell types. Unipotent cells are typically capable of differentiating into a single cell type. Non-embryonic stem cells are usually pluripotent or unipotent. Pluripotent stem cells are considered to be “lineage-restricted”, meaning that these stem cells can give rise to a cell committed to forming a particular limited range of differentiated cell types.

The primary cell lineages are endoderm cells, which include liver, intestine, pancreas, lung, and other internal organs; ectoderm cells, which include skin, hair, and neuronal cells; and mesoderm cells, which include hematopoietic, blood, muscle, cardiovascular, and bone cells. However, the primary lineages can be further restricted, for example, hematopoietic cells can be further restricted to myeloid or lymphoid lineages.

An “immature cell” is a cell that not fully specialized or differentiated. Cellular development is a multi-stage process. A fully immature cell (such as an embryonic stem cell) may pass through a few or many stages of development to become a mature, fully differentiated cell. Immature cells can be characterized by reduced or absent expression of genes, markers, and/or activity associated with mature cells. Immature cells encompassed by the invention include any cells, including intermediate cell types, that are less specialized or differentiated than a fully mature cell of the same cell type. As an example, an immature neuron represents a cell that is more differentiated than a neural stem cell, yet less differentiated than a mature neuron, and so represents an intermediate cell between these two types. As used herein, a “generated” or “induced” immature cell refers to any cell that is less differentiated than a fully mature cell of the same cell type, and/or is less differentiated relative to the maturity of the cell from which it was generated. Thus, for example, a pancreatic beta cell would be considered immature if it showed reduced expression of genes or activities associated with a mature beta cell, such reduced insulin expression/production or reduced glucose responsiveness.

Types of immature cells include: immature beta cells; immature neurons; blast cells including hemangioblasts, osteoblasts, myeloblasts, and erythroblasts; immature T-cells; immature B-cells; immature hepatic cells; immature cardiac cells; immature renal cells; immature gallbladder cells; immature intestinal cells; immature lung cells, and immature epithelial cells.

The term “differentiation” as used herein, refers to a developmental process whereby cells become specialized for a particular function, for example, where cells acquire one or more morphological characteristics and/or functions different from that of the initial cell type. The term “differentiation” includes both lineage commitment and differentiation of a cell into a mature, fully differentiated cell. Differentiation may assessed, for example, by monitoring the presence or absence of lineage markers, using immunohistochemistry or other procedures known to a worker skilled in the art. Differentiated progeny cells derived from progenitor cells may be, but are not necessarily, related to the same germ layer or tissue as the source tissue of the progenitor cells.

An “induced cell” is a cell that is produced or generated from a somatic cell, by reprogramming the somatic cell to alter its state of differentiation to become a pluripotent, multipotent, or immature cell. The term “somatic cell” includes any cell that is not itself a gamete, germ cell, gametocyte, or undifferentiated stem cell. Somatic cells are typically more differentiated than pluripotent, multipotent, or immature cells; thus, somatic cells must be reprogrammed to de-differentiate (that is, to become less differentiated and acquire one or more characteristics of a pluripotent, multipotent, or immature cell).

As used herein, “reprogramming” refers to a genetic process whereby differentiated somatic cells are converted into de-differentiated cells having a higher potency than the cells from which they were derived. A somatic cell is “reprogrammed” (directed to de-differentiate into a pluripotent, multipotent, or immature cell) according to the methods disclosed herein by contacting the somatic cell with one or more factors that alter the somatic cell's developmental program.

Specific stem/pluripotent cells that can be induced by the disclosed methods include neural stem cells, bone marrow stem cells, lung stem cells, kidney stem cells, endothelial stem cells, myocardial stem cells, muscle stem cells, bone cells, mesenchymal stem cells, hepatic stem cells, pancreatic stem cells, dermal stem cells, epidermal stem cells, and hematopoietic stem cells.

For example, somatic cells can be reprogrammed to generate neural stem cells (NSCs). It has been thought that the subventricular zone of the lateral ventricles and the dentate gyrus of the hippocampus are the main sources of human adult NSCs, which are considered to be a reservoir of new neural cells. Adult NSCs with potential neural capacity have also been isolated from white matter and inferior prefrontal subcortex in the human brain. Several references in stem cell biology have raised promising possibilities of replacing lost/damaged or degenerative neural cells by stem cell transplantation. However, sources of NSCs, sufficient quantities, and control of the differentiations for clinical uses represent a major barrier for transplantation. Thus, the generation of NSCs from non-brain sources has great therapeutic potential for treatment of various neural disorders.

An active fragment is a fragment of a protein, such as OCT4, which is capable of directing de-differentiation of a somatic cell into a pluripotent or immature cell. An active fragment would include the active region or functional domain, for example, an active fragment of a transcription factor would contain at least one or both of a DNA-binding domain and a co-factor binding site, while an active fragment of a ligand would contain at least a receptor binding/activation domain, and an active fragment of a receptor would contain at least one or both of an intracellular signaling domain and a ligand-binding domain. A derivative of OCT4 or the active fragment thereof is the protein or active fragment thereof which includes some modification, mutation, or addition, for example, including another chemical substance (such as polyethylene glycol), or which is associated with mutation such as addition, deletion, insertion or substitution of at least one, and preferably one to several amino acids. In other words, derivatives of OCT4, and active fragments thereof, include mutants, modified forms, and modification products of OCT4, and active fragments thereof, that are capable of directing de-differentiation of a somatic cell into a pluripotent or immature cell.

Expression of OCT4 within the somatic cell directs de-differentiation of the somatic cell into a multpotent or immature cell. The pluripotent or multipotent or immature cell type ultimately induced depends on the induction media used to generate the pluripotent or immature cell, as described in greater detail below.

OCT4 (octamer-binding transcription factor 4), also known as POU5F1 (POU domain, class 5, transcription factor 1), OCT3, or OTF3, is encoded by the POU5F1 gene. OCT4 is a POU family homeodomain transcription factor and is involved in the self-renewal of undifferentiated stem cells. Human OCT4 has at least two to five splice variant isoforms. As an example, the sequence for a specific human OCT4 variant, POU domain, class 5, transcription factor 1 isoform 1, is set forth in UniProtKB/Swiss-Prot Database Accession No. Q01860.

Functional derivatives and homologs of OCT4 are further contemplated for use in the disclosed methods. As used herein, a “functional derivative” is a molecule which possesses the capacity to perform the biological function of a molecule disclosed herein. For example, a functional derivative of OCT4 is a molecule that is able to functionally substitute for OCT4, e.g., in the reprogramming of ECs to HMLPs. Functional derivatives include fragments, parts, portions, equivalents, analogs, mutants, mimetics from natural, synthetic or recombinant sources including fusion proteins. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. Additions to amino acid sequences include fusions with other peptides, polypeptides or proteins.

A variant of a molecule is meant to refer to a molecule substantially similar in structure and function to either the entire molecule, or to a fragment thereof. Thus, as the term variant is used herein, two molecules are variants of one another if they possess a similar activity even if the structure of one of the molecules is not found in the other, or if the sequence of amino acid residues is not identical. The term variant includes, for example, splice variants or isoforms of a gene. Equivalents should be understood to include reference to molecules which can act as a functional analog or agonist. Equivalents may not necessarily be derived from the subject molecule but may share certain conformational similarities. Equivalents also include peptide mimics.

A “homolog” is a protein related to a second protein by descent from a common ancestral DNA sequence. A member of the same protein family (for example, the OCT family) can be a homolog. A “functional homolog” is a related protein or fragment thereof that is capable of performing the biological activity of the desired gene, i.e, is able to functionally substitute for OCT4 in the reprogramming of somatic cells to pluripotent or immature cells. Homologs and functional homologs contemplated herein include, but are not limited to, proteins derived from different species.

An OCT4 functional derivative or homolog can have 75%, 80%, 85%, 90%, 95% or greater amino acid sequence identity to a known OCT4 amino acid sequence, or 75%, 80%, 85%, 90%, 95% or greater amino acid sequence identity to a OCT4 family member or variant thereof. An OCT4 functional derivative or homolog can have, for example, 75%, 80%, 85%, 90%, 95% or greater amino acid sequence identity to UniProtKB/Swiss-Prot Database Accession No. Q01860.

Provided herein are vectors to drive expression of OCT4 for reprogramming. The vectors enable entry and expression of OCT4 in the somatic cell. A vector can be integrative, meaning it directs OCT4 to integrate into the somatic cell genome. A vector can also be non-integrative or episomal, meaning it enables expression of OCT4 from an extrachromosomal location. In either case, the vector is typically provided as a backbone vector into which the nucleic acid sequence for OCT4 is cloned by techniques known in the art.

Integrative vectors include retrovirus, lentivirus, adenovirus, adeno-associated virus, and other vectors that, once introduced into a cell, integrate into a chromosomal location within the genome of the subject and provide stable, long-term expression of the reprogramming factor. Exemplary vectors for stem cell induction are described, for example, in Yu J, et al., Science 318:1917-20 (2007) and Hanna J, et al., Cell 133:250-64 (2008). The nucleotide sequence of OCT4 can be cloned into the vector sequence, the vector is grown in appropriate host cells, and used to reprogram the somatic cell using the methods described in greater detail below.

Non-integrative vectors include episomal vectors, as well as engineered lentivirus vector variants that are non-integrative. These vectors direct expression of OCT4 as a separate genetic element. Because these vectors do not integrate into the chromosome, the risk of integration into a gene resulting in genetic harm or inactivation is avoided. The absence of chromosomal integration means that episomal vectors are more easily lost from the somatic cell; however, once the somatic cell is reprogrammed into a pluripotent or immature cell and delivered to a subject, the induced pluripotent or immature cell will be directed to re-differentiate within the tissue of the subject, and accordingly the vector is no longer needed.

Episomal vectors can be generated from, for example, BKV (BK polyoma virus), BPV-1 (bovine papillomavirus type 1), Epstein-Barr virus (EBV)-plasmid, EBV-BAC (bacterial artificial chromosome), EBNA-1 (Epstein-Barr nuclear antigen 1), scaffold matrix attachment region (S/MAR)-plasmid, S/MAR-BAC, Minichromosome, or human artificial chromosome (HAC)-based vectors. The vector also contains a multiple cloning site for introduction of the sequence of the reprogramming factor or factors, an EBV replication origin, and an EBNA-1 nuclear antigen, to permit extrachromosomal replication and expression in mammalian cells. References for episomal reprogramming of somatic cells are described, for example, in Meng X, et al, Mol Ther. 20:408-16 (2012); Okita K, et al., Stem Cells 31:458-66 (2013); and Yu J, et al., PLoS One 6:e17557 (2011). In a specific example, the episomal vector is, or is derived from or based on, a pCEP vector, such as pCEP1, pCEP2, pCEP3, or pCEP4.

The vector for expressing OCT4 comprises a strong promoter operably linked to the OCT4 gene. The phrase “operably linked” or “under transcriptional control” as used herein means that the promoter is in the correct location and orientation in relation to a polynucleotide to control the initiation of transcription by RNA polymerase and expression of the polynucleotide. In some embodiments, the promoter is an inducible promoter that allows one to control when the reprogramming factor is expressed. Suitable examples of inducible promoters include tetracycline-regulated promoters (tet on or tet off) and steroid-regulated promoters derived from glucocorticoid or estrogen receptors. Constitutive expression of TFs can be achieved using, for example, expression vectors with a SFFV or CAG (chicken beta-actin promoter with CMV enhancer) promoter. Inducible expression of TFs can be achieved using, for example, a tetracycline responsive promoter, such as the TRE3GV (Tet-response element 3rd generation) inducible promoter (Clontech Laboratories, Mountain View, Calif.). Alternatively, the promoter operably linked to the transgene may be a promoter that is activated in specific cell types and/or at particular points in development.

Depending on the promoter used, expression of OCT4 can be constitutive (continuous expression of the factor) or inducible (capable of being turned on and off). Expression can also be transient, that is, temporary expression of OCT4 over a limited time span. Transient expression may be achieved by use of a non-integrative vector, where the vector is lost from the cell or cell population over time, or by use of an inducible promoter in an integrative or non-integrative vector that can be manipulated to cease expression of the reprogramming gene after a period of time. In one embodiment, transient expression of OCT4 is employed to generate expression for no more than three days, no more than five days, no more than 10 days, or no more than one, two, or three weeks. Preferably, OCT4 expression is constitutive.

In the disclosed methods, OCT4 expression is driven by a strong or high expressing promoter. By “strong promoter” or “high expressing promoter” is meant a promoter that drives expression of OCT4 at above physiological levels. For example, a high expressing promoter can drive OCT4 protein or mRNA expression at two-fold, five-fold, ten-fold, fifteen-fold, twenty-fold, thirty-fold, forty-fold, fifty-fold, sixty-fold, eighty-fold, or one hundred-fold or greater levels, compared to physiological levels of OCT4 in a non-transformed somatic cell of the same cell type. Alternatively, a high expressing promoter can drive OCT4 protein or mRNA expression at two-fold, five-fold, ten-fold, fifteen-fold, twenty-fold, thirty-fold, forty-fold, fifty-fold, sixty-fold, eighty-fold, or one hundred-fold or greater levels, compared to levels of OCT4 driven by a less-strong or lower-expressing promoter. Examples of strong/high-expressing promoters include spleen focus-forming virus (SFFV) promoter and human elongation factor 1α (EF1) promoter. Examples of less-strong/lower-expressing promoters include the simian virus 40 (SV40) early promoter, cytomegalovirus (CMV) immediate-early promoter, Ubiquitin C (UBC) promoter, and the phosphoglycerate kinase 1 (PGK) promoter.

In a specific example, the vector is a lentiviral or episomal vector expressing OCT4 under control of the spleen focus-forming virus (SFFV) promoter. The vector may be modified such that a promoter present in the vector is replaced with the SFFV or EF1 promoter.

Suitable vectors can contain markers to identify and/or select transformed cells. Examples of selectable markers include visual markers such as green fluorescent protein (GFP), red fluorescent protein (RFP), or fluorescein; epitope markers such as His, c-myc, GST, Flag, or HA tags; enzymatic/nutritional markers such as DHFR (dihydrofolate reductase); or antibiotic resistance markers such as neomycin, puromycin, blasticidin, or hygromycin.

The disclosed methods for reprogramming can also involve culturing the human somatic cell in vitro or ex vivo with one or more, two or more, three or more, or four or more of these compounds.

In some embodiments, inventors have demonstrated that the high levels of OCT4 expression may be replaced with a combination of up to eight small molecules. These molecules are selected based on screening small molecules that target cellular mechanisms that are known to influence stem cell properties. These small molecules (8F, V6ACZBTU) include: valproic acid (“V”); 616452, a transforming growth factor-beta receptor inhibitor II (“6”); A-83-01(“A”) an ALK4, ALK5 and ALK7 inhibitor; CHIR99021 (“C”), a glycogen synthase kinase 3 inhibitor; DZNep/3-deazaneplanocin A (“Z”), a lysine methyltransferase EZH2 (KMT6) inhibitor; BIX01294 (“B”), a histone-lysine methyltransferase (HMTase) inhibitor; tranylcypromine hydrochloride (“T”), an inhibitor of the histone lysine demethylase LSD1; UNC0638 (“U”), a histone methyltransferase inhibitor (HMT, G9a/GLP selective methyltransferase chemical probe). These compounds are collectively referred to as “V6ACZBTU” or “8F”. Compounds “V6ACZB” are collectively referred to as “6F”.

In some embodiments, human amniotic fluid (AF) cells are reprogrammed to generate FLK+ multipotent stem cells or immature cells. FLK is also known as Fetal Liver Kinase-1 (FLK-1), Vascular Endothelial Growth Factor Receptor-2 (VEGFR-2), CD309, or Kinase Insert Domain Receptor (KDR). The UniProt Accession number for FLK is P35968. FLK+ cells are considered to be vascular progenitor cells that define the vascular and hematovascular lineages, capable of differentiating into endothelial cells, pericytes, vascular smooth muscle cells, hematopoietic cells, and cardiac cells.

To AF generate FLK+ multipotent stem cells or immature cells, mature cells are exposed to either 6F or 8F compounds described above with an appropriate concentration and cultured in MSC medium (DF12, 15% FBS, 10 ng/ml bFGF). Cells treated with either 6F or 8F chemicals proliferate in a normal rate but are slightly slower than control cells without exposing to chemical compounds. On day 7, almost all of the treated cells become multipotent cells or immature cells expressing FLK1 as compared to controls, which remain negative for FLK1. After washing away the 6F or 8F chemicals and then plating cells directly into the endothelial medium, EGM (Lonza, Biologics Inc.), cells tend to lose expression of FLK1, and differentiate into endothelial cells expressing CD31 and VE-Cadherin, specific markers for endothelial cells. The differentiated endothelial cells also bear an in vitro function activity such as AC-LDL (Acetylated-low density lipoprotein) uptake.

Accordingly, methods to reprogram a somatic cell can be as follows.

Somatic cells can be obtained from a biological sample. Sources of somatic cells that can be used to generate the desired stem cell include umbilical cord blood (UBC or CB), amniotic fluid (AF), bone marrow (BM), adipose tissue, blood, plasma, epidermal tissue, placenta, or any organ or tissue. Somatic cells can originate from various tissue or organ systems, including, but not limited to, blood, nerve, muscle, skin, gut, bone, kidney, liver, pancreas, thymus, and the like. In one example, the somatic cell is cord blood (CB) cell or a pancreatic islet cell.

Due to the ability of the reprogramming methods disclosed herein to reprogram any cell to induce the desired stem cell, even a heterologous population of cells can be essentially uniformly induced to generate a population of pluripotent or immature cells. Therefore, although cord blood and other biological samples can be further purified to obtain a single somatic cell type, according to the methods presented herein they do not need to be a pure population prior to inducing the desired stem cells.

If desired, different cell types can be fractionated into subpopulations. This may be accomplished using standard techniques for cell separation including, but not limited to, enzymatic treatment; cloning and selection of specific cell types, including but not limited to selection based on morphological and/or biochemical markers; selective growth of desired cells (positive selection), selective destruction of unwanted cells (negative selection); separation based upon differential cell agglutinability in the mixed population as, for example, with soybean agglutinin; freeze-thaw procedures; differential adherence properties of the cells in the mixed population; filtration; conventional and zonal centrifugation; centrifugal elutriation (counter-streaming centrifugation); unit gravity separation; countercurrent distribution; electrophoresis; fluorescence activated cell sorting (FACS); and the like.

Identifying the characteristics of a cell population can be performed upon or following isolation of a sample or expansion of somatic cells, prior to reprogramming. Alternatively, or in addition, cell typing can be performed after reprogramming, to determine the characteristics of the iSCs generated from reprogramming. Cells can be characterized by, for example, by growth characteristics (e.g., population doubling capability, doubling time, passages to senescence), karyotype analysis (e.g., normal karyotype; maternal or neonatal lineage), flow cytometry (e.g., FACS analysis), immunohistochemistry and/or immunocytochemistry (e.g., for detection of epitopes), gene expression profiling (e.g., gene chip arrays; polymerase chain reaction (for example, reverse transcriptase PCR, real time PCR, and conventional PCR)), protein arrays, protein secretion (e.g., by plasma clotting assay or analysis of PDC-conditioned medium, for example, by Enzyme Linked ImmunoSorbent Assay (ELISA)), mixed lymphocyte reaction (e.g., as measure of stimulation of PBMCs), and/or other methods known in the art.

Isolated cells, or untreated samples such as CB, can be used to initiate cell cultures. Cells or samples are transferred to sterile tissue culture vessels either uncoated or coated with extracellular matrix or ligands such as laminin, collagen (native, denatured or crosslinked), gelatin, fibronectin, or other extracellular matrix proteins. Cells are cultured in any culture medium capable of sustaining growth of the cells such as, but not limited to, Dulbecco's modified Eagle's medium (DMEM), advanced DMEM, DMEM/MCDB 201, Eagle's basal medium, Ham's F10 medium (F10), Ham's F-12 medium (F12), DF-12 (DMEM plus Ham's F12), DMEM/F12, Hayflick's Medium, Iscove's modified Dulbecco's medium, Mesenchymal Stem Cell Growth Medium (MSCGM), RPMI 1640, STEMSPAN, and CELL-GRO-FREE. The culture medium can be supplemented with one or more components including, for example fetal bovine serum, preferably about 2-15% (v/v); equine serum; human serum; fetal calf serum; beta-mercaptoethanol, preferably about 0.001% (v/v); one or more growth factors, for example, platelet-derived growth factor (PDGF), epidermal growth factor (EGF), fibroblast growth factor (FGF), vascular endothelial growth factor (VEGF), insulin-like growth factor-1 (IGF-1), leukocyte inhibitory factor (LIF) and erythropoietin; amino acids, including L-valine; and one or more antibiotic and/or antimycotic agents to control microbial contamination, such as, for example, penicillin G, streptomycin sulfate, amphotericin B, gentamicin, and nystatin, either alone or in combination.

Stem cells may be expanded in serum-free medium comprising bovine serum albumin, human insulin, human transferrin, 2-mercaptoethanol, Iscove's modified Dulbecco's medium, and supplemented with one or more of thrombopoietin, Flt-3 ligand, stem cell factor/steel factor, IL-3, IL-6, IL-9, granulocyte colony-stimulating factor, and nerve growth factor. An example of a suitable serum-free medium for stem cell expansion is STEMSPAN medium (STEMCELL Technologies, Vancouver, BC, Canada).

The somatic cells can be cultured to expand the cell numbers, prior to reprogramming. Sufficient numbers of somatic cells may be isolated in the initial sample; however, even if an acceptable number of somatic cells is present in the initial sample, expansion of the cells in culture can provide an even greater supply of somatic cells for reprogramming. Methods of culturing and expanding somatic cells are known in the art. See, for example, Helgason et al., Basic Cell Culture Protocols, 4th Edition, Human Press Publishing, 2013; and Mitry et al, Human Cell Culture Protocols, 3rd Edition, Human Press Publishing, 2012.

Once sufficient numbers of somatic cells are generated, the somatic cells are seeded onto tissue culture plates, in the range of 5,000 to 25,000 cells per cm². In a specific example, 10,000 to 20,000 cells per cm² are seeded onto a tissue culture plate or flask that is coated with laminin, collagen, gelatin, fibronectin, or other extracellular matrix proteins.

As a first step in the reprogramming process, the somatic cells are transduced with a vector driving expression of OCT4 under the control of a high expressing promoter, for a sufficient time and under conditions that allow the induction factor to gain entry into the somatic cells and reprogram them to de-differentiate. Sufficient time can be 1 hour to 1 week, or 2, 4, 6, 8, 10, 12 hrs, or 1 to 3 days. Conditions depend in part on the vector and growth media used, as well as the type of cell desired to be generated. Exemplary conditions for reprogramming are disclosed in the following references:

Integrative vector culture conditions: see, i.e., Yu J, et al., Science 318:1917-20 (2007) and Hanna J, et al., Cell 133:250-64 (2008).

Non-integrative/episomal culture conditions: see, i.e., Meng X, et al, Mol Ther. 20:408-16 (2012); Okita K, et al., Stem Cells 31:458-66 (2013); and Yu J, et al., PLoS One 6:e17557 (2011).

Following transduction, the de-differentiated somatic cell is cultured in specialized medium and conditions designed to produce the desired progenitor or immature cell. Examples of media and growth conditions that can be utilized to produce specific cell types are as follows:

Cells are induced to lineage-restricted stem/progenitor cells under a tissue or cell type specialized medium. The method would be used to direct reprogramming with lineage specialized stem cells using the OCT4 transcriptional factor, such as neural stem cells, skin stem cells, liver stem cell, pancreatic stem cells, bone marrow stem cells, lung stem cells, heart stem cells, kidney stem cells, endothelial stem cells, and mesenchymal stem cells. As an example, CD34+ cells are transduced with OCT4 for two or three days and the transduced cells are then placed in a neural stem cell medium to induce OCT4 expressing cells into neural stem/progenitor cells. In another example, CD34+ cells are transduced with OCT4 and then placed in an embryonic stem cell medium to induce OCT4 expressing cells into hemangioblasts.

In a particular example, CD34+CB cells are reprogrammed to generate induced neural stem cells (iNSCs). Somatic cells are transduced with a vector as described above and cultured in human neural stem cells (NSC) medium: ReNcell medium (Millipore) supplemented with 20 ng/ml human FGF-2 and 20 ng/ml human EGF (PerproTech) on day 3. NSC medium can be changed daily. The cells were treated with accutase and passaged to laminin coated tissue culture plates on day 7-9. The CB induced NSC (CB-iNSC) were then passaged every 5 days when the cells reached 80-90% confluence.

In another particular example, CD34+CB cells are reprogrammed to generate induced hemangioblasts. Somatic cells are transduced with a vector as described above and cultured in embryonic stem cell (ES cell) medium, such as DMEM/DF12 supplemented with 20% KNOCK-OUT serum replacement (GIBCO), 2 mM L-glutamine, 0.1 mM non-essential amino acids, 10 ng/ml human bFGF and 100 U/ml penicillin/streptomycin.

One benefit of the methods disclosed herein is that the stem cells can be generated from culture of somatic cells in a “feeder free” system; that is, the somatic cells can be cultured to generate stem cells in the absence of a feeder cell layer.

Feeder cell layers are adherent, growth-arrested but viable cells that are cultured to form a bottom layer on which other cells are grown in a co-culture system. Feeder cell layers provide an extracellular matrix and secrete known and unknown factors into the medium. Many mammalian cell types, such as stem cells, will not survive or proliferate without physical contact with a feeder layer. As such, feeder cells, typically mouse or human fibroblasts, are often required in stem cell culture methods. However, the presence of feeder cells is a detriment to establishing clinical grade stem cells, which for use in humans must be produced without any animal cells or products. The methods provided herein allow cell reprogramming without the use of feeder cells.

This disclosure further provides a multipotent or immature cell produced by any of the disclosed methods. The pluripotent or immature cell can be, for example, an induced neural stem cell (iNSC), a hemangioblast/hematopoietic stem cell, a megakaryocyte, or an immature pancreatic beta cell.

The present invention further provides a method for repairing or regenerating a tissue or differentiated cell lineage in a subject. The method involves obtaining an iSC from a somatic cell and administering the iSC to a subject, e.g., a subject having a myocardial infarction, congestive heart failure, stroke, ischemia, peripheral vascular disease, alcoholic liver disease, cirrhosis, Parkinson's disease, Alzheimer's disease, diabetes, cancer, arthritis, an internal or external wound, immunodeficiency, anemia including aplastic anemia, or a genetic disorder, or other diseases or conditions where an increase or replacement of a particular cell type/tissue, or cellular re-differentiation is desirable.

The term neurodegenerative condition (or disorder) is an inclusive term encompassing acute and chronic conditions, disorders or diseases of the central or peripheral nervous system. A neurodegenerative condition may be age-related, or it may result from injury or trauma, or it may be related to a specific disease or disorder. Acute neurodegenerative conditions include, but are not limited to, conditions associated with neuronal cell death or compromise including cerebrovascular insufficiency, focal or diffuse brain trauma, diffuse brain damage, spinal cord injury or peripheral nerve trauma, e.g., resulting from physical or chemical burns, deep cuts or limb severance. Examples of acute neurodegenerative disorders are: cerebral ischemia or infarction including embolic occlusion and thrombotic occlusion, reperfusion following acute ischemia, perinatal hypoxic-ischemic injury, cardiac arrest, as well as intracranial hemorrhage of any type (such as epidural, subdural, subarachnoid and intracerebral), and intracranial and intravertebral lesions (such as contusion, penetration, shear, compression and laceration), as well as whiplash and shaken infant syndrome. Chronic neurodegenerative conditions include, but are not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome), and prion diseases (including, but not limited to Creutzfeldt-Jakob, Gerstmann-Straussler-Scheinker disease, Kuru and fatal familial insomnia), demyelination diseases and disorders including multiple sclerosis and hereditary diseases such as leukodystrophies.

The iSCs can be used for autologous (i.e., cells are obtained from the same subject to be treated with the reprogrammed stem cells), allogeneic (i.e., cells are obtained from another subject of the same species as the subject to be treated), or xenogeneic (i.e., cells are obtained from a subject of a different species from the subject to be treated) transplantation.

Some non-limiting examples of damage that can be repaired and reversed by the invention include surgical removal of any portion (or all) of the diseased or damaged organ or tissue, drug-induced damage, toxin-induced damage, radiation-induced damage, environmental exposure-induced damage, sonic damage, heat damage, hypoxic damage, oxidation damage, viral damage, age or senescence-related damage, inflammation-induced damage, immune cell-induced damage, for example, transplant rejection, immune complex-induced damage, and the like.

As used herein, the terms “subject” and “patient” are used interchangeably and refer to an animal, including mammals such as non-primates (e.g., cows, pigs, horses, cats, dogs, rats etc.) and primates (e.g., monkey and human).

The terms “treatment”, “treating”, and the like, as used herein include amelioration or elimination of a developed disease or condition once it has been established or alleviation of the characteristic symptoms of such disease or condition. As used herein these terms also encompass, depending on the condition of the patient, preventing the onset of a disease or condition or of symptoms associated with a disease or condition, including reducing the severity of a disease or condition or symptoms associated therewith prior to affliction with said disease or condition. Such prevention or reduction prior to affliction refers to administration of iSCs to a patient that is not at the time of administration afflicted with the disease or condition. “Preventing” also encompasses preventing the recurrence or relapse-prevention of a disease or condition or of symptoms associated therewith, for instance after a period of improvement.

The cells can be administered as a pharmaceutical/therapeutic cell composition that comprises a pharmaceutically-acceptable carrier and iSCs as described and exemplified herein. In one example, therapeutic cell compositions can comprise AF cells induced to differentiate along a neural pathway or lineage. The therapeutic cell compositions can comprise cells or cell products that stimulate cells in the patient's tissue requiring regeneration to divide, differentiate, or both. It is preferred that the therapeutic cell composition induce, facilitate, or sustain repair and/or regeneration of the damaged or diseased tissues or organs in the patient to which they are administered.

The cells can be administered to the patient by injection. For example, the cells can be injected directly into the damaged tissue of the patient, or can be injected onto the surface of the tissue, into an adjacent area, or even to a more remote area with subsequent migration to the patient's tissue requiring regeneration or repair. In some preferred aspects, the cells can home to the diseased or damaged area.

The cells can also be administered in the form of a device such as a matrix-cell complex. Matrices include biocompatible scaffolds, lattices, self-assembling structures and the like, whether bioabsorbable or not, liquid, gel, or solid. Such matrices are known in the arts of therapeutic cell treatment, surgical repair, tissue engineering, and wound healing. The cells of the invention can also be seeded onto three-dimensional matrices, such as scaffolds and implanted in vivo, where the seeded cells may proliferate on or in the framework, or help to establish replacement tissue in vivo with or without cooperation of other cells. Also contemplated are matrix-cell complexes in which the cells are growing in close association with the matrix and when used therapeutically, growth, repair, and/or regeneration of the patient's own damaged tissue is stimulated and supported, and proper angiogenesis is similarly stimulated or supported. The matrix-cell compositions can be introduced into a patient's body in any way known in the art, including but not limited to implantation, injection, surgical attachment, transplantation with other tissue, and the like.

A successful treatment could thus comprise treatment of a patient with a disease, pathology, or trauma to a body part with a therapeutic cell composition comprising iSCs, in the presence or absence of another cell type. For example, and not by way of limitation, the cells preferably at least partially integrate, multiply, or survive in the patient. In other preferred embodiments, the patient experiences benefits from the therapy, for example from the ability of the cells to support the growth of other cells, including stem cells or progenitor cells present in the damaged or diseased tissue, from the tissue in-growth or vascularization of the tissue, and from the presence of beneficial cellular factors, chemokines, cytokines and the like, but the cells do not integrate or multiply in the patient. In some aspects, the patient benefits from the therapeutic treatment with the cells, but the cells do not survive for a prolonged period in the patient. For example, in one embodiment, the cells gradually decline in number, viability or biochemical activity. In other embodiments, the decline in cells may be preceded by a period of activity, for example growth, division, or biochemical activity.

The administering is preferably in vivo by transplanting, implanting, injecting, fusing, delivering via catheter, or providing as a matrix-cell complex, or any other means known in the art for providing cell therapy.

### Reprogramming Pancreatic Cells

Further provided herein are methods to reprogram mature pancreatic islet cells to stem cells for pancreatic beta (β) cells. The methods involve differentiation or reprogramming mature pancreatic cells so that pancreatic β cells return to a more primitive developmental state of stem-like cells. In some embodiments, the resulting stem cells or immature cells by dedifferentiation or reprogramming gain unlimited self-renewal functions, which allow generating pancreatic β cells in a large scale for therapy and avoiding or reducing the risk of tumor formation.

The methods involve transduction of pancreatic cells with an integrative or episomal vector driving expression of OCT4 under control of a high-expressing promoter, such as SFFV and EF. These methods are disclosed in detail above. Following transduction, cells are cultured in a medium appropriate for the growth of pancreatic cells, and/or in medium appropriate for the growth of embryonic stem cells.

Insulin-producing, glucose sensitive β cells are characterized in part by expression of PDX1 (pancreatic and duodenal homeobox 1), a nuclear protein involved in development of the pancreas that plays a role in glucose-dependent regulation of insulin gene expression. PDX1 expression is correlated with expression of the cell surface marker CD24; thus CD24 identifies PDX1-positive beta cells. Other markers for beta cell progenitors include Hlxb9, Sox9 and Nkx6-1. Markers for maturing beta cells are C-peptide and proinsulin or insulin.

In a specific example, primary β cells are transduced with OCT4 lentiviruses followed by culturing in beta cell medium, then culturing in 1:1 mix of beta cell medium and hES cell medium (DMEM/F12 supplemented with 20% knockout serum replacement, 1 mM L-glutamine, 0.1 mM nonessential amino acids, 0.1 mM 2-mercaptoethanol, and 8 ng/ml recombinant human fibroblast growth factor-basic), and finally culturing with hES cell medium. Reprogrammed colonies can be observed two to three weeks (14 to 21 days) after viral infection. By three to four weeks (21 to 30 days) after transduction, CD24+ pancreatic progenitors are generated. These CD24+ pancreatic progenitors are negative for specific markers for ES cells. CD24+ pancreatic progenitor cells can then be expanded, for example, in matrigel coated plates in 1:1 mix of hES cell medium and MEF (mouse embryonic fibroblasts) conditioned medium. MEF conditioned medium supports the growth of isolated clones.

In another specific example, fresh, un-passaged islet beta cells are transduced with OCT4 lentiviruses followed by culturing in beta cell medium as described above. Approximately 10 reprogramming colonies are observed day 7 post-transduction in 1×10³ β-cells. The clones are observed only in the cells transduced with lentiviruses expressing OCT4 under either the SFFV or EF promoter. There is no any clone seen when OCT4 expression is controlled under a week expressing promoter, CMV promoter. It apparently shortens the time it takes to form a clone when fresh, unmassaged islet cells are used. All reprogrammed clones became strongly positive for FLK1 10 days after transduction. CXCR4 expression is also seen in the FLK1 positive clones. The FLK1 and CXCR4 positive cells derived from reprogramming are associated with endoderm derivatives, which can differentiate to beta cells.

The expanded cells can be further differentiated to functional islet beta cells using protocols as disclosed, for example, in WO/2011/109837, the contents of which are incorporated by reference herein.

### Expansion of Stem Cells or Progenitor Cells Using Small Molecules

Based on small molecules targeting cellular mechanisms that are known to affect stem cell functions, inventors have screened these molecules and a small molecule is selected if it meets the criteria: 1) CD34+ cells isolated from bone marrow or cord blood are cultured in the HSC medium with a small molecule and after 5 day culture cell number is increased by 20-, 30 or 100-fold; 2) expanded cells are composed of a substantial portion of cells expressing markers, CD34+CD38- or CD34+CD38+.

This disclosure further provides methods of expansion of a stem cell or immature cell by contacting with one or more compounds selected from: a transforming growth factor-beta receptor (TGF-βR) inhibitor, a lysine-specific demethylase 1 (LSD1) inhibitor, a cAMP agonist, a histone lysine methyltransferase (enhancer of zeste homolog-2/EZH2) inhibitor, and a histone methyltransferase (HMTase) G9a inhibitor. The methods can involve culturing the human stem cell or immature cell in vitro or ex vivo with one or more, two or more, three or more, or four or more of these compounds. In some embodiments, the stem or immature cell is a CD34+ cell.

More particularly, chemical small molecules in an appropriate concentration cultured in association with hematopoietic CD34+ cells drive these cell proliferation or expansion and retain significant fractions of hematopoietic stem cells and progenitor cells. In some embodiments, hematopoietic CD34+ cells contain short and long-term engraftment stem cells for bone marrow transplantation. These are isolated from one of sources: human bone marrow, peripheral blood of G-CSF-mobilized donors, human core blood, fetal liver and placenta. Hematopoietic short and long-term engraftment cells are tightly associated with markers, CD34+CD38+ and CD34+CD38−, respectively.

According to the method of the present invention, the inventors have determined which compound is more favorable expansion of a stem cell or progenitor cell in an appropriate medium. In some embodiments, inventors have screened numerous small molecules modulating different cellular mechanisms. Only a very small subset of modulators appear to expand or retain a significant proportion of hematopoietic CD34+CD38− population ex vivo, and the CD34+CD38− population is associated with long-term engraftment of bone marrow stem cell transplantation.

In a specific embodiment, CD34+ cells isolated from peripheral blood of G-CSF-mobilized donors are cultured for 11 days under minimal cytokines (SCF, TPO, and Flt-3 ligand) and different compounds, C, 6, F and Z. Chemical C (GSK3 inhibitor XVI) alone rapidly proliferates bone marrow CD34 cells leading to their maturation at a significantly rapid rate compared to control, but does not have much of effect on either CD34+CD38- or CD34+CD38-population. Chemical F (a cAMP agonist) can induce a significant proportion of CD34+CD38+ population while chemical 6 and Z each individually show the best effect enhancing both CD34+CD38- and CD34+ and CD38+ positive cells.

In an additional embodiment, the inventors have determined the effect of a combination of chemical compound 6 and Z on the CD34+ cells isolated from bone marrows. After a 6 day culture under chemicals 6Z, the population of CD34+CD38− rises to approximately 28-fold of that the input compared to the 10-fold increase of the un-treated cells. In addition, compounds-treated cells better retain CD34+CD38− markers (˜41% in the treated cells vs ˜4% in the untreated cells) and result in a higher percentage of the CD34+CD38− retention than that of input cells. A similar observation is seen when chemicals 6 and Z are used to treat human umbilical cord blood (CB). Culture of human CB CD34+ cells for 6 days with chemicals 6 and Z results in a 28-fold increase in CD34+CD38− cells compared with input cells while this population seen in untreated cells is only increased by 16 fold.

Chemical small molecules screens have shown that Bix (BIX01294) at 1 uM retained a large portion of stem/progenitor cells with a marker, CD34+CD38− cells as compared to that of the control when this molecule is added to CB CD34+ cells cultured for 5 days in HSC media containing SCF, TPO, and Flt-3 ligand at 100 ng/ml each. Z at 1× (100 nM) or Bix at 1 uM retained a large portion of stem/progenitor cells with a marker, CD34+CD38− cells as compared to that of the control. When combined both together, there was a synergistic effect on remaining a significant proportion of CD34+CD38-population of nearly 62% after 5 day culture. Trihydrochloride hydrate is a histone lysine methyltranferase inhibitor while Z (DZNep), 3-Deazaneplanocin A is a lysine mehtyltransferase EZH2 inhibitor.

LSD1, a histone lysine demethylase, has been shown to play a role in the repressive effects of SALL4 on expansion of hematopoietic stem/progenitor cells (Aquila et al, Blood, 2011, 118:576-85). The inventors screened the effect of LSD1 inhibitors on the expansion of hematopoietic precursor cells. CB CD34+ cells are cultured for 5 days in HSC media containing SCF, TPO, and Flt-3 ligand at 100 ng/ml each. Tranylcypromine hydrochloride (T), LSD1 inhibitor V at 10 uM retained a significant fraction of CD34+CD38− cells and expanded this population, as compared to that of the control. When compared to the input, T treatment resulted in an approximately 60 and 45-fold increase in CD34+CD38− cells and total cell counts, respectively after 5 day culture. Other LSD1 inhibitors, I, II, and III appeared to have no or little effect on the expansion of hematopoietic stem/progenitor cells.

The inventors have also determined the dose for BIX, Z and UNC on the expansion of hematopoietic stem/progenitor cells. There is no significant toxicity at concentrations of 0.25×. UNC (UNC0638) and BIX are quite similar in their impact of expansion of hematopoietic stem/progenitor cells. There is a synergistic effect on expansion of hematopoietic stem/progenitor cells when combining UNC or BIX with Z. UNC is UNC0638 hydrate, a histone methyltransferase inhibitor (HMT) and selective inhibitor of G9a and GLP histone lysine methyltransferases.

Therapy for aplastic anemia, marrow failure, leucopenia and ARS would be possible by the use of small molecules as disclosed herein to enhance the growth of marrow cells or expand hematopoietic stem cells in situ. For example, inventors have demonstrated that administration of 616452 enhances the blood and marrow recovery from leukopenia, particularly thrombocytopenia resulting from the chemotherapy.

Genomic editing of ex vivo or in vitro cells is further contemplated. Genomic editing involves inserting, replacing, or removing DNA from a genome using artificially engineered nucleases to create specific double-stranded breaks at desired locations in the genome and alter the genome as desired. The cell's endogenous mechanisms then repair the induced break by natural processes of homologous recombination and nonhomologous end-joining. There are currently four families of engineered nucleases being used: zinc finger nucleases, transcription activator-like effector nucleases, the CRISPR/Cas system (Shalem et al., Science 343:84-87 (2014)), and engineered meganuclease re-engineered homing endonucleases.

This disclosure further provides a multipotent or immature cell produced by any of the disclosed methods. The pluripotent or immature cell can be, for example, an induced neural stem cell (iNSC), a hemangioblast/hematopoietic stem cell, a megakaryocyte, or an immature pancreatic beta cell.

The present invention further provides a method for repairing or regenerating a tissue or differentiated cell lineage in a subject. The method involves obtaining an iSC from a somatic cell and administering the iSC to a subject, e.g., a subject having a myocardial infarction, congestive heart failure, stroke, ischemia, peripheral vascular disease, alcoholic liver disease, cirrhosis, Parkinson's disease, Alzheimer's disease, diabetes, cancer, arthritis, an internal or external wound, immunodeficiency, anemia including aplastic anemia, or a genetic disorder, or other diseases or conditions where an increase or replacement of a particular cell type/tissue, or cellular re-differentiation is desirable.

The term neurodegenerative condition (or disorder) is an inclusive term encompassing acute and chronic conditions, disorders or diseases of the central or peripheral nervous system. A neurodegenerative condition may be age-related, or it may result from injury or trauma, or it may be related to a specific disease or disorder. Acute neurodegenerative conditions include, but are not limited to, conditions associated with neuronal cell death or compromise including cerebrovascular insufficiency, focal or diffuse brain trauma, diffuse brain damage, spinal cord injury or peripheral nerve trauma, e.g., resulting from physical or chemical burns, deep cuts or limb severance. Examples of acute neurodegenerative disorders are: cerebral ischemia or infarction including embolic occlusion and thrombotic occlusion, reperfusion following acute ischemia, perinatal hypoxic-ischemic injury, cardiac arrest, as well as intracranial hemorrhage of any type (such as epidural, subdural, subarachnoid and intracerebral), and intracranial and intravertebral lesions (such as contusion, penetration, shear, compression and laceration), as well as whiplash and shaken infant syndrome. Chronic neurodegenerative conditions include, but are not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome), and prion diseases (including, but not limited to Creutzfeldt-Jakob, Gerstmann-Straussler-Scheinker disease, Kuru and fatal familial insomnia), demyelination diseases and disorders including multiple sclerosis and hereditary diseases such as leukodystrophies.

Any cells generated by the disclosed methods can be used for autologous (i.e., cells are obtained from the same subject to be treated with the reprogrammed stem cells), allogeneic (i.e., cells are obtained from another subject of the same species as the subject to be treated), or xenogeneic (i.e., cells are obtained from a subject of a different species from the subject to be treated) transplantation.

Some non-limiting examples of damage that can be repaired and reversed by the invention include surgical removal of any portion (or all) of the diseased or damaged organ or tissue, drug-induced damage, toxin-induced damage, radiation-induced damage, environmental exposure-induced damage, sonic damage, heat damage, hypoxic damage, oxidation damage, viral damage, age or senescence-related damage, inflammation-induced damage, immune cell-induced damage, for example, transplant rejection and the like.

As used herein, the terms “subject” and “patient” are used interchangeably and refer to an animal, including mammals such as non-primates (e.g., cows, pigs, horses, cats, dogs, rats etc.) and primates (e.g., monkey and human).

The terms “treatment”, “treating”, and the like, as used herein include amelioration or elimination of a developed disease or condition once it has been established or alleviation of the characteristic symptoms of such disease or condition. As used herein these terms also encompass, depending on the condition of the patient, preventing the onset of a disease or condition or of symptoms associated with a disease or condition, including reducing the severity of a disease or condition or symptoms associated therewith prior to affliction with said disease or condition. Such prevention or reduction prior to affliction refers to administration of iSCs to a patient that is not at the time of administration afflicted with the disease or condition. “Preventing” also encompasses preventing the recurrence or relapse-prevention of a disease or condition or of symptoms associated therewith, for instance after a period of improvement.

The cells can be administered as a pharmaceutical/therapeutic cell composition that comprises a pharmaceutically-acceptable carrier and iSCs as described and exemplified herein. In one example, therapeutic cell compositions can comprise AF cells induced to differentiate along a neural pathway or lineage. The therapeutic cell compositions can comprise cells or cell products that stimulate cells in the patient's tissue requiring regeneration to divide, differentiate, or both. It is preferred that the therapeutic cell composition induce, facilitate, or sustain repair and/or regeneration of the damaged or diseased tissues or organs in the patient to which they are administered.

The cells can be administered to the patient by injection. For example, the cells can be injected directly into the damaged tissue of the patient, or can be injected onto the surface of the tissue, into an adjacent area, or even to a more remote area with subsequent migration to the patient's tissue requiring regeneration or repair. In some preferred aspects, the cells can home to the diseased or damaged area.

The cells can also be administered in the form of a device such as a matrix-cell complex. Matrices include biocompatible scaffolds, lattices, self-assembling structures and the like, whether bioabsorbable or not, liquid, gel, or solid. Such matrices are known in the arts of therapeutic cell treatment, surgical repair, tissue engineering, and wound healing. The cells of the invention can also be seeded onto three-dimensional matrices, such as scaffolds and implanted in vivo, where the seeded cells may proliferate on or in the framework, or help to establish replacement tissue in vivo with or without cooperation of other cells. Also contemplated are matrix-cell complexes in which the cells are growing in close association with the matrix and when used therapeutically, growth, repair, and/or regeneration of the patient's own damaged tissue is stimulated and supported, and proper angiogenesis is similarly stimulated or supported. The matrix-cell compositions can be introduced into a patient's body in any way known in the art, including but not limited to implantation, injection, surgical attachment, transplantation with other tissue, and the like.

A successful treatment could thus comprise treatment of a patient with a disease, pathology, or trauma to a body part with a therapeutic cell composition comprising iSCs, in the presence or absence of another cell type. For example, and not by way of limitation, the cells preferably at least partially integrate, multiply, or survive in the patient. In other preferred embodiments, the patient experiences benefits from the therapy, for example from the ability of the cells to support the growth of other cells, including stem cells or progenitor cells present in the damaged or diseased tissue, from the tissue in-growth or vascularization of the tissue, and from the presence of beneficial cellular factors, chemokines, cytokines and the like, but the cells do not integrate or multiply in the patient. In some aspects, the patient benefits from the therapeutic treatment with the cells, but the cells do not survive for a prolonged period in the patient. For example, in one embodiment, the cells gradually decline in number, viability or biochemical activity. In other embodiments, the decline in cells may be preceded by a period of activity, for example growth, division, or biochemical activity.

The administering is preferably in vivo by transplanting, implanting, injecting, fusing, delivering via catheter, or providing as a matrix-cell complex, or any other means known in the art for providing cell therapy.

The inventors have determined that multipotent or immature cells can be generated by contacting human somatic cells with at least one glycogen synthase kinase 3 (GSK-3) inhibitor, alone or in combination with additional molecules disclosed herein.

In some embodiments, the GSK-3 inhibitor is CHIR99021 or a functional derivative thereof. CHIR99021 (also referred to herein as “C”) is 6-[[2-[[4-(2,4-dichlorophenyl)-5-(5-methyl-1H-imidazol-2-yl)-2 pyrimidinyl]amino]ethyl]amino]-3-pyridinecarbonitrile, with the structure:

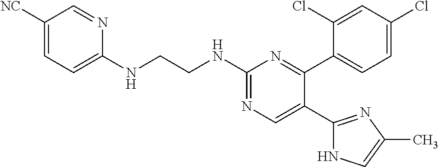 [SMILES: Cc1c[nH]c(-c2cnc(NCCNc3ccc(C#N)cn3)nc2-c2ccc(Cl)cc2Cl)n1]

The inventors have further determined that multipotent or immature cells can be generated by contacting human somatic cells with at least one transforming growth factor-beta receptor (TGF-βR) inhibitor, alone or in combination with additional molecules disclosed herein. TGF-β receptors include Type I receptors, such as ALK1 (activin receptor-like kinase-1), ALK2, ALK7, TGF-βR1/ALK5 (activin receptor-like kinase-5), Activin A type 1B receptor (ACVR1B)/ALK4, Bone morphogenetic protein receptor, type 1A (BMPR1A)/ALK3, and BMPR1B/ALK6. TGF-β receptors also include Type II receptors, such as TGF-βR2, ACVR2, ACVR2B, BMPR2, and anti-Mullerian hormone receptor, type II (AMHR2). Transforming growth factor-beta receptor1 kinase inhibitors (TGF-βR inhibitors) include inhibitors I, II, III, IV, V, VI and VII. These inhibitors bear different chemical structures.

An example of a TGF-βR inhibitor I is [3-(Pyridin-2-yl)-4-(4-quinonyl)]-1H-pyrazole. An example of a TGF-βR inhibitor II is 2-(3-(6-Methylpyridin-2-yl)-1H-pyrazol-4-yl)-1,5-naphthyridine. An example of a TGF-βR inhibitor III is 2-(5-Benzo[1,3]dioxol-4-yl-2-tert-butyl-1H-imidazol-4-yl)-6-methylpyridine. An example of a TGF-βR inhibitor IV is 3-(6-Methylpyridin-2-yl)-4-(4-quinolyl)-1-phenylthiocarbamoyl-1H-pyrazole. An example of a TGF-βR inhibitor V is 2-(5-Chloro-2-fluorophenyl)pteridin-4-yl)pyridin-4-yl amine. An example of a TGF-βR inhibitor VII is 1-(2-((6,7-Dimethoxy-4-quinolyl)oxy)-(4,5-dimethylphenyl)-1-ethanone.

In some embodiments, the TGF-βR inhibitor is an inhibitor of ALK 5 such as “616452” or a functional derivative of 616452 thereof. 616452 (also referred to herein as “6”) is 2-[3-(6-methyl-2-pyridinyl)-1H-pyrazol-4-yl]-1,5-naphthyridine, with the structure:

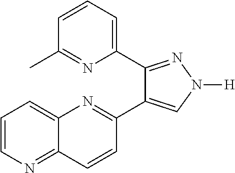 [SMILES: Cc1cccc(-c2n[nH]cc2-c2ccc3ncccc3n2)n1]

In another embodiment, the TGF-βR inhibitor is the ALK inhibitor identified as “616454” (also referred to herein as “4”) or a functional derivative thereof. “616454” is 3-(6-Methylpyridin-2-yl)-4-(4-quinolyl)-1-phenylthiocarbamoyl-1H-pyrazole, with the structure:

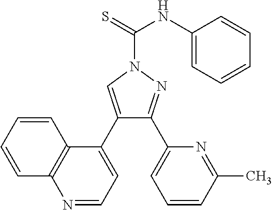 [SMILES: Cc1cccc(-c2nn(C(=S)Nc3ccccc3)cc2-c2ccnc3ccccc23)n1]

In another embodiment, the TGF-βR inhibitor is the ALK inhibitor identified as “SB 431452” (also referred to herein as “5”) or a functional derivative thereof. SB 431452 is 4-[4-(1,3-benzodioxol-5-yl)-5-(2-pyridinyl)-1H-imidazol-2-yl]benzamide, with the structure:

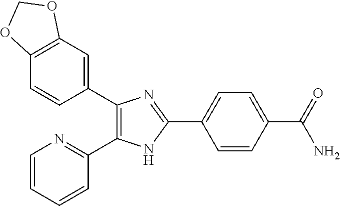 [SMILES: NC(=O)c1ccc(-c2nc(-c3ccc4c(c3)OCO4)c(-c3ccccn3)[nH]2)cc1]

In another embodiment, the TGF-βR inhibitor is the ALK4, ALK5, and ALK7 inhibitor “A-83-01” (“A”), or a functional derivative thereof. A-83-01 is 3-(6-Methyl-2-pyridinyl)-N-phenyl-4-(4-quinolinyl)-1H-pyrazole-1-carbothioamide with the structure:

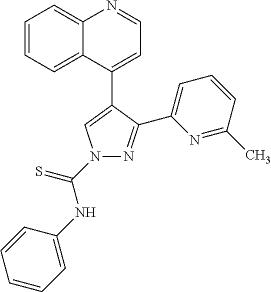 [SMILES: Cc1cccc(-c2nn(C(=S)Nc3ccccc3)cc2-c2ccnc3ccccc23)n1]

The inventors have further determined that multipotent or immature cells can be generated by contacting human somatic cells with at least one lysine-specific demethylase 1 (LSD1) inhibitor, alone or in combination with additional molecules disclosed herein.

In some embodiments, the inhibitor of LSD1 is tranylcypromine hydrochloride (LSD1 inhibitor V) or a functional derivative thereof. Tranylcypromine hydrochloride (also referred to herein as “T”) is (+)-trans-2-Phenylcyclopropylamine hydrochloride, with the structure:

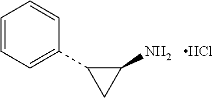

In some embodiments, LSD1 inhibitor IV is a cell-permeable tranylcypromine analog that acts as a potent, irreversible inhibitor of lysine specific demethylase 1 or a functional derivative thereof. LSD1 inhibitor IV (also referred to herein as “T4”) is 2-(1R,2S)-2-(4-(Benzyloxy)phenyl)cyclopropylamino)-1-(4-methylpiperazin-1-yl)ethanone with the structure:

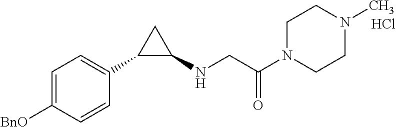 [SMILES: CN1CCN(C(=O)CN[C@@H]2C[C@H]2c2ccc(OCc3ccccc3)cc2)CC1 ; Cl]

The inventors have further determined that multipotent or immature cells can be generated by contacting human somatic cells with at least one histone lysine methyltransferase (EZH2) inhibitor, alone or in combination with additional molecules disclosed herein.

In some embodiments, the EZH2 (enhancer of zeste homolog 2) inhibitor is 3-deazaneplanocin A or a functional derivative thereof. 3-deazaneplanocin A/DZNep (also referred to herein as “Z”) is 5R-(4-amino-1H-imidazo[4,5-c]pyridin-1-yl)-3-(hydroxymethyl)-3-cyclopentene-1S,2R-diol, with the structure:

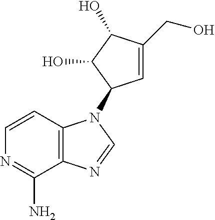 [SMILES: Nc1nccc2c1ncn2[C@@H]1C=C(CO)[C@@H](O)[C@H]1O]

The inventors have further determined that multipotent or immature cells can be generated by contacting human somatic cells with at least one histone methyltransferase (HMTase) G9a inhibitor, alone or in combination with additional molecules disclosed herein.

In some embodiments, the HMTase G9a inhibitor is BIX 01294 or a functional derivative thereof. Bix 01294 (also referred to herein as “Bix”) is 2-(Hexahydro-4-methyl-1H-1,4-diazepin-1-yl)-6,7-dimethoxy-N-[1-(phenylmethyl)-4-piperidinyl]-4-quinazolinamine trihydrochloride hydrate, with the structure:

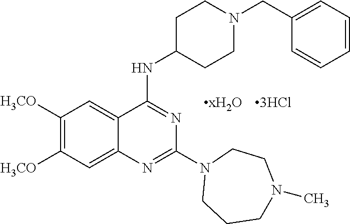 [SMILES: COc1cc2nc(N3CCCN(C)CC3)nc(NC3CCN(Cc4ccccc4)CC3)c2cc1OC]

G9a/KMT1C and GLP/G9a-like protein/Eu-HMTase1/KMT1D are members of the Suv39h subgroup of SET domain-containing molecules. In some embodiments, an inhibitor of G9a and GLP histone lysine methyltransferases, UNC 0638 (also referred to herein as “UNC”) is 2-Cyclohexyl-6-methoxy-N-[1-(1-methylethyl)-4-piperidinyl]-7-[3-(1-pyrrolidinyl)propoxy]-4-quinazolinamine, with the structure:

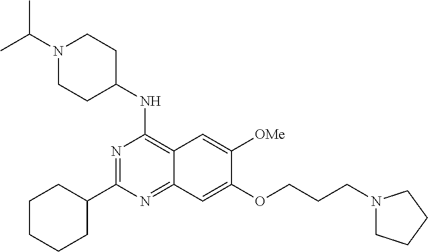 [SMILES: COc1cc2c(NC3CCN(C(C)C)CC3)nc(C3CCCCC3)nc2cc1OCCCN1CCCC1]

The inventors have further determined that multipotent or immature cells can be generated by contacting human somatic cells with at least one histone deacetylase (HDAC) inhibitor, alone or in combination with additional molecules disclosed herein.

In some embodiments, the HDAC inhibitor is valproic acid. Valproic acid (“V”) is sodium 2-propylpentanoate, with the structure:

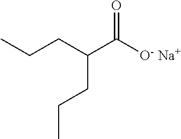

Multipotent or immature cells can further be generated by contacting human somatic cells with at least one JAK (Janus kinase) inhibitor, alone or in combination with additional molecules disclosed herein.

The inventors have further determined that multipotent or immature cells can be generated by contacting human somatic cells with at least one cAMP agonist, alone or in combination with additional molecules disclosed herein.

In some embodiments, the cAMP agonist is forskolin or a functional derivative thereof. Forskolin/FSK (“F”) is 7β-acetoxy-8,13-epoxy-1α,6β,9α-trihydroxylabd-14-en-11-one, with the structure:

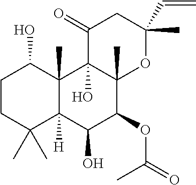

In a specific embodiment, the human somatic cell is a CD34+ cell cultured in vitro or ex vivo with a combination of a TGF-βR inhibitor, such as 616452 or a functional derivative thereof, and an EZH2 inhibitor, such as 3-deazaneplanocin A (DZNep) or a functional derivative thereof. The inventors have discovered that a TGF-βR inhibitor II and a histone methyltransferase inhibitor, DZNep, form a synergistic combination to expand hematopoietic stem/progenitor cell populations.

In another specific embodiment, the human somatic cell is a CD34+ cell cultured in vitro or ex vivo with a combination of: a GSK-3 inhibitor, such as CHIR99021 or a functional derivative thereof; a TGF-βR inhibitor, such as 616452 or a functional derivative thereof; a cAMP agonist, such as forskolin or a functional derivative thereof, and an EZH2 inhibitor, such as 3-deazaneplanocin A or a functional derivative thereof.

In another embodiment, the human somatic cell is a CD34+ cell cultured in vitro or ex vivo with a combination of an EZH2 inhibitor, such as 3-deazaneplanocin A or a functional derivative thereof, and an HMTase G9a inhibitor, such as BIX 01294 or a functional derivative thereof.

In another embodiment, the human somatic cell is a CD34+ cell cultured in vitro or ex vivo in embryonic stem cell medium. In a specific embodiment, the CD34+ cell is cultured with a TGF-βR1 inhibitor, such as 616452 or a functional derivative thereof. In a further embodiment, a CD34+ cell is cultured with a combination of two or all three TGF-βR inhibitors selected from: 616454 or a functional derivative thereof; SB 431452 or a functional derivative thereof; and 616452 or a functional derivative thereof.

The compounds would also be useful in the medical treatment of diseases that involve hematopoietic stem/progenitor cells. The disclosed method may also be used to regeneration of bone marrow cells in subject after administration of compounds described above.

In a specific embodiment, administration of 616452 with an appropriate dose in a subject in vivo dramatically enhances white blood cell recovery particularly platelets.

The barrier to expanded use of CB is limited numbers of hematopoietic stem/progenitor cells per cord at harvest. As cell dose has been shown to be a major determinant of engraftment and survival after CB transplantation, low stem cell numbers represents the most significant barrier to successful CB stem cell transplantation.

The ability to expand ex vivo, prior to transplantation, the stem cell components of a single cord blood unit will greatly increase the viability of this treatment modality. Infusing patients with larger numbers of stem cells as opposed the limited cells available in an unexpanded cord blood unit, should greatly increase the likelihood of successful engraftment.

A single cord blood unit as a source of cells for transplantation is given only to children weighing less than 30 kg; multiple units must be given to adults to achieve successful engraftment. An expansion of high quality cells of even 5- to 10-fold prior to transplantation would eliminate the need for multiple cord blood units, and improve engraftment rates in older children and adults.

The expansion of non-hematopoietic adult stem cells or precursor cells, including stem cells or precursor cells isolated from organs such as brain, heart, liver, pancreas, kidney, lung, etc., has important clinical applications, particularly as an external source of cells for tissue or organ repairing or replacement.

This invention provides a method for expanding a stem/progenitor cell population ex vivo, the method comprising the identification of small molecules with the expansion enhancement activity in an amount effective to expand the stem/progenitor cell population ex vivo. In a specific embodiment, the stem/progenitor cells are hematopoietic cells.

The expanded cells would be used for treatment or prophylaxis of diseases, disorders, or abnormalities in a subject requiring a stem cell or an expanded stem cell derived therefrom.

### Enhancement of Megakaryocyte Maturation and Platelet Production by Small Molecules

The inventors herein demonstrate successfully shortened megakaryocyte maturation and enhancement of platelet production under suitable conditions in the presence of one or more small chemical molecules. In some embodiments, the methods may include contacting hematopoietic stem/progenitor cells or megakaryocyte cells, and an effective amount of a compound.

The disclosure features methods for inducing polyploidization and increase the size of megakaryocytes, and promoting differentiation of megakaryocytes to platelets. The disclosed methods would be used for treating bone marrow and blood disorders, such as adult or neonatal thrombocytopenia, myelodysplastic syndromes, chronic myeloproliferative disorders and acute myeloid leukemia. The methods would also be unitized for enhancing the platelet recovery after cord blood stem cell transplantation or chemotherapy.

In some embodiments, inventors have demonstrated that a small chemical molecule stimulates or modulates the SALL4 expression. In a specific embodiment, the small molecule is 616452, TGF βR1 kinase inhibitor II. SALL4 has been disclosed to be a robust stimulator for the expansion of hematopoietic stem cells and progenitor cells (US/2010/376122).

In some embodiments, inventors have also demonstrated 616452, as a megakaryocyte (MK) inducer in bone marrow CD34+ cells. CD34+ cells isolated from bone marrow or mobilized peripheral blood in the presence of 616452 at 10 μM is cultured with thrombopoietin (TPO), stem cell factor (SCF, also known as Kit-ligand, the ligand for the c-Kit receptor), and IL-3. In such a condition for 4 days, a significant number of large megakaryocytes appeared while the control induced with the compound solvent, only appears to have one or two large megakaryocytes. Live cell staining and immunofluorescence revealed all the large cells are positive for CD41, a marker unique to megakaryocytes.

In a specific embodiment, CD34+ cells isolated from human umbilical CB is cultured in HSC media induced with 616452 with the control induced with DMSO, the compound solvent. The appearance of larger cells characteristic of developing megakaryocytes appears after 4 days of culture. The quantity and size of cells increase over time. By 8 days, the culture dishes are predominately composed of megakaryocytes and the formation of megakaryocytic clusters begins. A Giemsa-Wright stain of these cells confirms the morphology of megakaryocytes.

In another embodiment, within one week of culture for CB CD34+ cells, megakaryocyte progenitor cells in an appropriate condition described above, the chemical, 616452 is able to increase a 10 to 200-fold of polyploidization, the process required for megakaryocyte maturation with exhibiting the accumulation of DNA content, to 64N or greater. In some embodiments, CB CD34+ cells are from pre-term or neonates and megakaryocyte (MK) ploidy correlates with their maturation and platelet production.

The disclosure provides methods of promoting or shortening fetal or neonatal megakaryocyte cell maturation. In some embodiments, retardation of megakaryocyte maturation is one of main causes of poor megakaryocyte engraftment after cord blood transplantation, and neonatal thrombocytopenia. The disclosed methods may be utilized for resolving these issues.

The disclosure further provides method of simulating hematopoietic recovery or regeneration, particularly platelets by administering 616452 in an appropriate dose to a subject with deficiencies of hematopoietic cells due to a stress such as chemotherapy, radiation, aging and disorders.

The inventors used C57BL/6J mice to administrate with SFU, a chemotherapy agent (150 mg/kg, intraperitoneal injection). Peripheral blood (100 ul each) from injected mice is collected for assessment of the peripheral blood count using a hematology analyzer. When while blood cell count, particularly a platelet count, is very low at day 6, mice are administrated with a small chemical molecule, 616452 (10 mg/kg) every other day for a total of three doses. The next analysis reveals that many of the mice show increases in platelet counts while the control mice, injected with DMSO/PBS solution, continued to decrease in platelet counts. By day 12, the control mice begin to recover, while the chemical-induced mice become thrombocytosis, which platelet counts are through the roof, with almost twice as many platelets as normal mice. In addition, chemotherapy treated mice after 616442 injection show significant improvement or recovery of the white blood cell count as well.

The disclosed methods described above can benefit the following clinical situations:
(1) other drugs such as Neurogenic® or Neulasta® have been used for supporting the levels of neutrophils for patients with leukopenia. 616452 can provide additional benefits by simultaneous administration of Neupogen® or Neulasta® or other their functional derivatives. Such benefits to patients with leukopenia can be achieved by administration of 616452 alone as well.
(2) Thrombocytopenia affects 20-30% (25,000-30,000 per year) of infants admitted to the neonatal intensive care unit. Approximately 9% of those infants are severe and experience clinically significant bleeding (usually intracranial). Platelet transfusions are the only therapeutic option for thrombocytopenic neonates. Recent studies have shown that megakaryocytes of neonates are smaller and have lower ploidy than those of adults. Administration of 616452 to neonates with thrombocytopenia can provide benefits to increased megakaryocyte size and ploidy in response to increased platelet consumption might underlie the predisposition of sick neonates to thrombocytopenia.
(3) Human umbilical cord blood is an important stem cell source for patients who lack other suitable donors. However, slower platelet engraftment is a major drawback of CB transplantation. Platelet engraftment takes an average of approximately 50 days for CB recipients, versus 20 days for mobilized peripheral blood cells derived from adult donors. TPO stimulates the megakaryocyte formation in vivo, but it does not shorten its maturation time. Post-transplant admiration of 616452 can accelerate platelet reconstitution following bone marrow transplantation, autologous or allogeneic bone marrow transplantation, respectively, in myeloablated hosts.
(4) Differentiation therapy has emerged as a powerful way to target specific hematologic malignancies. Administration of 616452 can benefit patients with acute myeloid leukemias, myelodysplastic syndromes and chronic myeloproliferative disorders by inducing terminal differentiation of megakaryocytes.

Inventors have screened other TGFβ inhibitors, such as 616454, SB431542 and LY364947. It appears that these compounds have little or no effect on the increased number of large megakaryocytes from CB or shortened their maturation. CB CD34+ cells are cultured for 5 days in HSC media containing SCF, TPO, and Flt-3 ligand at 100 ng/ml each.

The disclosed methods may be utilized for treating bone marrow disorders. Exemplary bone marrow disorders include acute megakaryocytic leukemia, myelodysplastic syndromes, chronic myeloid leukemia, multiple myeloma, and chronic myeloproliferative disorders. Delayed platelet engraftment is a major complication of umbilical cord blood transplantation. Megakaryocytes derived from umbilical cord blood in vitro are smaller than megakaryocytes derived from bone marrow (BM) or mobilized peripheral blood from adults. Small megakaryocyte size may contribute to delayed platelet engraftment. Expansion of these under the influence of compound 6 before transplant may offer promise for improved platelet recovery.

The disclosed methods could be used to co-treat with JAK2 or JAK3 inhibitors in order to obtain the better treatment effects. Examples of JAK2 and/or JAK3 inhibitors include Cucurbitacin, Lestaurtinib, AG 490, TCS 21311, SD 1008, ZM 39923 hydrochloride, CP 690550 citrate, NSC 33994, ZM 449829, WHI-P 154, and 1,2,3,4,5,6-Hexabromocyclohexane.

The invention of the present application also relates to the proliferation and/or differentiation of hematopoietic precursor cells derived from the umbilical cord blood or marrow into megakaryocytes. The disclosed methods could also be used to improve the platelet recovery when umbilical cord blood is treated with transforming growth factor-beta receptor inhibitors, particularly transforming growth factor-beta receptor inhibitor II.

The disclosed methods could also be used to treat with one or more other agents simultaneously. These could include a granulocyte colony-stimulating factor (G-CSF), and agents related to the increase of megakaryocyte ploidy, such as nicotinamide (NIC; vitamin B3), Src inhibitor (3Z)—N,N-Dimethyl-2-oxo-3-(4,5,6,7-tetrahydro-1H-indol-2-ylmethylidene)-2,3-dihydro-1H-indole-5-sulfonamide (SI; Su6656); aurora B inhibitor N-(4-(6-methoxy-7-(3-morpholinopropoxy)quinazolin-4-ylamino)phenyl)benzamide (ABI; ZM447439) and Rho-Rock inhibitor (RRI).

The disclosed methods include contacting megakaryocyte cells and an effective amount of a compound, which enhances megakaryocyte progenitor cell expansion or megakaryocyte differentiation, promoting polyploidization and drastically shortening megakaryocyte maturation. In a specific embodiment, a compound is “616452” or a functional derivative thereof. 616452 (also referred to herein as “6”) is 2-[3-(6-methyl-2-pyridinyl)-1H-pyrazol-4-yl]-1,5-naphthyridine with a Formula below:

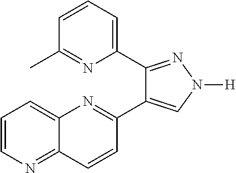 [SMILES: Cc1cccc(-c2n[nH]cc2-c2ccc3ncccc3n2)n1]

As used herein, the terms “subject” and “patient” are used interchangeably and refer to an animal, including mammals such as non-primates (e.g., cows, pigs, horses, cats, dogs, rats etc.) and primates (e.g., monkey and human).

The terms “treatment”, “treating”, and the like, as used herein include amelioration or elimination of a developed disease or condition once it has been established or alleviation of the characteristic symptoms of such disease or condition. As used herein these terms also encompass, depending on the condition of the patient, preventing the onset of a disease or condition or of symptoms associated with a disease or condition, including reducing the severity of a disease or condition or symptoms associated therewith prior to affliction with said disease or condition. Such prevention or reduction prior to affliction refers to administration of iSCs to a patient that is not at the time of administration afflicted with the disease or condition. “Preventing” also encompasses preventing the recurrence or relapse-prevention of a disease or condition or of symptoms associated therewith, for instance after a period of improvement.

The cells can be administered as a pharmaceutical/therapeutic cell composition that comprises a pharmaceutically-acceptable carrier and iSCs as described and exemplified herein. In one example, therapeutic cell compositions can comprise AF cells induced to differentiate along a neural pathway or lineage. The therapeutic cell compositions can comprise cells or cell products that stimulate cells in the patient's tissue requiring regeneration to divide, differentiate, or both. It is preferred that the therapeutic cell composition induce, facilitate, or sustain repair and/or regeneration of the damaged or diseased tissues or organs in the patient to which they are administered.

The cells can be administered to the patient by injection. For example, the cells can be injected directly into the damaged tissue of the patient, or can be injected onto the surface of the tissue, into an adjacent area, or even to a more remote area with subsequent migration to the patient's tissue requiring regeneration or repair. In some preferred aspects, the cells can home to the diseased or damaged area.

The cells can also be administered in the form of a device such as a matrix-cell complex. Matrices include biocompatible scaffolds, lattices, self-assembling structures and the like, whether bioabsorbable or not, liquid, gel, or solid. Such matrices are known in the arts of therapeutic cell treatment, surgical repair, tissue engineering, and wound healing. The cells of the invention can also be seeded onto three-dimensional matrices, such as scaffolds and implanted in vivo, where the seeded cells may proliferate on or in the framework, or help to establish replacement tissue in vivo with or without cooperation of other cells. Also contemplated are matrix-cell complexes in which the cells are growing in close association with the matrix and when used therapeutically, growth, repair, and/or regeneration of the patient's own damaged tissue is stimulated and supported, and proper angiogenesis is similarly stimulated or supported. The matrix-cell compositions can be introduced into a patient's body in any way known in the art, including but not limited to implantation, injection, surgical attachment, transplantation with other tissue, and the like.

A successful treatment could thus comprise treatment of a patient with a disease, pathology, or trauma to a body part with a therapeutic cell composition comprising iSCs, in the presence or absence of another cell type. For example, and not by way of limitation, the cells preferably at least partially integrate, multiply, or survive in the patient. In other preferred embodiments, the patient experiences benefits from the therapy, for example from the ability of the cells to support the growth of other cells, including stem cells or progenitor cells present in the damaged or diseased tissue, from the tissue in-growth or vascularization of the tissue, and from the presence of beneficial cellular factors, chemokines, cytokines and the like, but the cells do not integrate or multiply in the patient. In some aspects, the patient benefits from the therapeutic treatment with the cells, but the cells do not survive for a prolonged period in the patient. For example, in one embodiment, the cells gradually decline in number, viability or biochemical activity. In other embodiments, the decline in cells may be preceded by a period of activity, for example growth, division, or biochemical activity.

The administering is preferably in vivo by transplanting, implanting, injecting, fusing, delivering via catheter, or providing as a matrix-cell complex, or any other means known in the art for providing cell therapy.

The present disclosure is further illustrated by the following non-limiting examples. The contents of all references cited herein are incorporated by reference in their entirety.

### EXAMPLES

### Cell Cultures

CD34+ Cell Isolation.

CD34+ cells were isolated from cord blood or from peripheral blood of G-CSF-mobilized adult donors and cultured as follows. Mononuclear cells (MNCs) were isolated using a simple red blood cell lysis (15 minutes at room temperature using BD PharmLyse) or using Ficoll-Paque density gradient centrifugation (Jaatinen and Laine, Current Protocols Stem Cell Biol. 1:2A.1.1-2A.1.4. (2007)). The cells were then incubated with MACS CD34+ Microbead kit (Miltenyi Biotec, Auburn, Calif.) and run through a magnetic column resulting in selection for CD34+ cells. On average the percentage of CD34+ cells obtained from any given isolation had a purity range of 90-95% CD34+ cells. Human cortex neural stem cell (hcx NSC) cells were purchased from Millipore.

CD34+ cells were cultured in STEMSPAN medium (STEMCELL Technologies, Vancouver, BC, Canada) supplemented with 10% FBS, 100 ng/ml hSCF (human stem cell factor), 100 ng/ml hTPO (human thrombopoietin), 100 ng/ml hFLT-3 ligand (Human Fms-related Tyrosine Kinase 3) (Peprotech, Rocky Hill, N.J., USA) and 100 U/ml penicillin/streptomycin (Gibco). Human cortex Neural Progenitor Cell line (hcx NSC) was maintained in ReNcell medium: ReNcell NSC Maintenance medium (Millipore) supplemented with 20 ng/ml human EGF, 20 ng/ml human bFGF (Peprotech) and 100 U/ml penicillin/streptomycin. For expansion of cells targeted for cell sorting, we used the same media containing a different cytokine cocktail of 100 ng/ml SCF, TPO and 10 ng/ml IL-3.

Promoters for lentiviral and episomal plasmids were replaced with the spleen focus forming virus (SFFV) promoter sequence (SEQ ID NO: 1). Promoters for lentiviral and episomal plasmids were replaced with the EF1 promoter sequence (SEQ ID NO: 2).

### Reprogramming CB CD34 Cells to Hemangioblasts Via Lentivirus

Lentiviruses carrying the human OCT4 gene or a control GFP gene, each under control of the spleen focus forming virus (SFFV) promoter, were packaged and produced using 293FT cell line. The viruses were concentrated by centrifugation and stored at −80 C. Lentiviruses were added to cells at a MOI of 2-5 in the presence of 8 μg/ml polybrene (Millipore) for 4 to 6 hours (Day 0). Then, transduced cells were washed with warm PBS and seeded at a density of approximately 20,000/mm² in tissue culture plates that were pre-coated with 10 μg/ml fibronectin. The next day (Day 1), half of the growth medium was removed and replaced with an equal amount of embryonic stem cell (ES cell) medium (DMEM/DF12 supplemented with 20% KNOCK-OUT serum replacement (GIBCO), 2 mM L-glutamine, 0.1 mM non-essential amino acids, 10 ng/ml human bFGF and 100 U/ml penicillin/streptomycin). On day 2, the medium was changed to ES cell medium completely and refreshed every 2 days. On day 7, the adherent cells reached confluency and were replated at a density of 50,000/mm² after Accutase (Millipore) digestion.

### Reprogramming CB CD34+ Cells to Hemangioblasts Using an Episomal Plasmid

1-5×10⁶ CD34+ cells were transduced with EBNV1-based pCEP4-OCT4 expression plasmid by electroporation using Human CD34 Cell Nucleofector® Kit (Lonza, Koln, Germany). The transduced cells were incubated with ES cell medium under hypoxia (5% O₂) and were allowed to recover for 1 day before following the lentivirus reprogramming strategy as previously mentioned.

Reprogramming of CD34+ Cells to NSCs with a Lentivirus Vector

Lentiviruses carrying the human OCT4 gene or a control GFP gene, each under control of the spleen focus forming virus (SFFV) promoter, were packaged and produced using 293FT cell line (Aguila et al, 2011, Blood, 576-586). The viruses were concentrated by centrifugation and stored at −80° C. Lentiviruses were added to cultured CD34+ cells at a MOI of 2-5 in the presence of 8 μg/ml polybrene (Millipore) for 4 to 6 hours (Day 0). Then, transduced cells were washed by warm PBS and seeded at a density of 20,000/mm² in tissue culture plates that were pre-coated with 10 μg/ml fibronectin. Media during the transduction and initial seeding was in hematopoietic stem cell medium consisting of primarily: StemSpan, 10% FBS, 1×P/S, 100 ng/ml of SCF, TPO and Flt3-Ligand. The next day (Day 1), half of the growth medium was replaced with DMEM/F12 medium (DMEM/DF12 with 15% FBS, 10 ng/ml human bFGF and 100 U/ml penicillin/streptomycin). On day 2, the medium was changed to DMEM/F12 medium completely and refreshed every 2 days. On day 7, the adherent cells reached confluence and were replated at a density of 50,000/mm² after Accutase (Millipore) digestion to dissociate cells. When the cells reached confluence again, all the cells were dissociated into single cells and plated at a density of 50,000/mm² on laminin (20 μg/ml) coated plates in ReNcell medium (Millipore). The reprogrammed cells (cord blood induced neural stem cells, CB-iNSCs, or adult CD34+ induced neural stem cells, CD34-iNSCs) were passaged every 3-4 days with a doubling time of approximately 24 hours. ReNcell medium were refreshed every 2-3 days. For neurosphere formation, the CB-iNSCs were seeded in low attachment (non-coated) petri dishes at a density of 200,000/ml in ReNcell medium. During the reprogramming procedure, 3 μM CHIR99021 (Stemgent) was added to the culture medium starting from the time of lentiviral transduction until the second passage in ReNcell medium. CHIR99021 was used at 3 uM as stated in the above paragraph. It was dissolved in DMSO at a stock concentration of 3 mM (1000×). The stock was added fresh into the medium at every media change.

### Transduction of CD34+ Cells by Episome Infection

For reprogramming of CD34+ cells to iNSCs by an OCT4 episomal vector, the CD34+ cells were transduced with an EBNV1-based pCEP4-OCT4 expression plasmid in which the CMV promoter is replaced by the SFFV promoter. Cells were transduced by electroporation using the Human CD34 Cell NUCLEOFECTOR® Kit (Lonza, Koln, Germany). Post-transduction, cells were incubated with hypoxia (5% 02), following the lentivirus reprogramming strategy described above. Dzep (3-deazaneplanocin, Sigma) was added to the solution after the first passage in ReNcell medium to maintain the cells for a longer period before senescence/quiescence.

### Lentiviral Production and Reprogramming of Human Islet β-Cells

Lentiviruses for OCT4 transduction of beta cells were produced independently after co-transfecting the 293T cell line in 150 mm dishes with lentiviral vectors expressing OCT4 controlled by either the SFFV or EF1 promoter. Viral supernatant was concentrated and filtered through a 0.20 μm filter (Millipore). One day before infection, primary beta cells, T0199 (Applied Biological Materials, Inc) were seeded in 24-well tissue culture plates on collagen at a density of 5×10³ cells/well and transduced with OCT4 lentiviruses in the presence of 8 ng/ml polybrene and Prigrow I medium supplemented with fetal bovine serum to a final concentration of 10% and Penicillin/Streptomycin (PIMs) (Applied Biological Materials Inc). After incubating for 6 hours, media was replaced with PIMs for first 24 hours, then replaced with 1:1 mix of PIMs and hES cell medium (DMEM/F12 supplemented with 20% knockout serum replacement, 1 mM L-glutamine, 0.1 mM nonessential amino acids, 0.1 mM 2-mercaptoethanol, and 8 ng/ml recombinant human fibroblast growth factor-basic) for the next 24 hours, and finally replaced with hES cell medium.

About 20 small reprogrammed colonies were observed in each 5×10³ beta cell seeded well day 17 to 19 after viral infection. By day 28-30 after transduction, a specific surface marker for pancreatic progenitors, PDX1, became positive. These cells were also negative for specific markers for ES cells (TRA-1-60). By day 30-33 post transduction colonies were picked up and cells were rapidly expanded in matrigel coated plates in 1:1 mix of hES cell medium and MEF (mouse embryonic fibroblasts) conditioned medium. MEF conditioned medium greatly supports the growth of isolated clones.

### Isolation of Cell Types for Further Differentiation

Cells were isolated for nestin (NSCs) and FLK-1 (hemangioblasts) before further differentiation. For cell sorting, cells were centrifuged for 5 minutes at 200 G at 4° C. and resuspended with 25 μl of CD41-FITC antibody for 30 minutes on ice. Cells are then washed gently with PBS and centrifuged for 5 minutes at 200 G at 4° C. Cells are then suspended at a concentration of 10⁶ cells/ml of PBS containing 2% FBS. Cells are sorted via FACS Aria.

Differentiation of iNSC

For random differentiation and maturation into three neural cell lineages, CD34+-iNSC cells were cultured in PLO/Laminin coated glass coverslips in ReNcell medium without bFGF and EGF for 14 days, or with addition of BDNF, GDNF, and Forskolin in the medium. For specific differentiation, CD34+-iNSC cells were induced by addition of 20 ng/ml BDNF and 20 ng/ml GDNF (PerproTech).

Hemangioblast differentiation is done by addition of 8-Br-cAMP (100 uM) and TGF-Beta inhibitor SB 431542 (10 uM) was used to enhance the efficiency of reprogramming into endothelial. Hemangioblasts were generated without these two compounds, but during the differentiation into endothelial cells, these two molecules were used to increase the efficiency of reprogramming.

### Small Molecule Reprogramming

CD34+ cells were induced using 616452 at a concentration of 10 μM dissolved in DMSO for 4 days before the appearance of large megakaryocytes. 616452 (also called RepSox), was purchased separately from Biovision and Millipore. The chemical is highly unstable above room temperature and in the presence of light and therefore needs to be changed every two to three days. A fresh aliquot of the chemical was used at every media change and the final concentration in media is 10 μM. CD34+ cells showed no significant change until after 4 days of induction with the chemical, at which point large megakaryocyte-like cells began to appear.

### Flow Cytometry Analysis

Cells were centrifuged for 5 minutes at 200 G at 4° C. Cells were resuspended with CD34, 38 and/or 41 conjugated antibodies for 30 minutes on ice. Cells were then washed with 2 ml of PBS and centrifuged for 5 minutes at 200 G at 4° C. The final pellet was resuspended using 300 μl of 2% formalin.

### Real-Time PCR

Total RNA was extracted by ALLPREP DNA/RNA Mini Kit (Qiagen) and cDNA was synthesized using QuantiTect Rev. Transcription Kit (Qiagen). PCR amplification was conducted with PLATINUM PCR Supermix, High fidelity (Life Technologies). Quantitative PCR (qPCR) was run on 7300 Real-Time PCR System (Applied Biosystems) with Power SYBR Green PCR Master Mix (Life Technologies). All qPCR were conducted in triplicate. The expression results of real-time PCR were presented by log₂^{fold} according to the Δ(ΔCt) which is normalized to ACTB expression. Detailed information on all primers is provided in Table 1.

TABLE 1
Primers used for PCR and Quantitative PCR

| | | | Size |
|---|---|---|---|
| Gene Name | Sense Primer (5′>3′) | Anti-sense Primer (5′>3′) | (bp) |
| Nestin (NES) | AAGACTTCCCTCAGCTTT | GGAGCAAAGATCCAAGAC | 85 |
| | (SEQ ID NO: 3) | (SEQ ID NO: 4) | |
| SOX2 | CGAGTGGAAACTTTTGTC | CAGCGTGTACTTATCCTT | 151 |
| | (SEQ ID NO: 5) | (SEQ ID NO: 6) | |
| Musashi | GACTCGAACGAAGAAGAT | ATGTCCTCACTCTCAAAC | 171 |
| 1(MSI1) | (SEQ ID NO: 7) | (SEQ ID NO: 8) | |
| SOX1 | GTAGTTGTTACCGCTCTT | GAAATGCTCAGATACATAAAGT | 126 |
| | (SEQ ID NO: 9) | (SEQ ID NO: 10) | |
| PAX6 | TGAAGCAAGAATACAGGTAT | GGAATTGGTTGGTAGACA | 150 |
| | (SEQ ID NO: 11) | (SEQ ID NO: 12) | |
| CD34 | TCCCACTAAACCCTATACA | CTCTGATGCCTGAACATT | 116 |
| | (SEQ ID NO: 13) | (SEQ ID NO: 14) | |
| CD38 | ATGTGATGCTCAATGGAT | AGTCTCTGGAATCTTCTCT | 144 |
| | (SEQ ID NO: 15) | (SEQ ID NO: 16) | |
| CD45 | GCTTAAACTCTTGGCATTT | TTTGAGGTTTGGTGACTT | 136 |
| | (SEQ ID NO: 17) | (SEQ ID NO: 18) | |
| OCT4 | GGTTCTATTTGGGAAGGTA | ATACTGGTTCGCTTTCTC | 195 |
| | (SEQ ID NO: 19) | (SEQ ID NO: 20) | |
| GBX2 | GGCAAGGGAAAGACGAGTCA | GGGTCTTCCTCCTTGTGAGC | 133 |
| | (SEQ ID NO: 21) | (SEQ ID NO: 22) | |
| HOXA2 | ACCCAGTGCAAGGAAAACCA | ACCTGGCAAACTGGGTGAAA | 435 |
| | (SEQ ID NO: 23) | (SEQ ID NO: 24) | |
| HOXB6 | GACCCGCTGAGACATTACCC | TGTTGCACGAATTCATCCGC | 316 |
| | (SEQ ID NO: 25) | (SEQ ID NO: 26) | |
| HOXB2 | CGCCAGGATTCACCTTTCCT | TTCCTCGGAAAAAGGGACCG | 126 |
| | (SEQ ID NO: 27) | (SEQ ID NO: 28) | |
| FOXG1 | GGCAAGGGCAACTACTGGAT | CTGAGTCAACACGGAGCTGT | 294 |
| | (SEQ ID NO: 29) | (SEQ ID NO: 30) | |
| PAX2 | TGTGACTGGTCGTGACATGG | CTAGTGGCGGTCATAGGCAG | 271 |
| | (SEQ ID NO: 31) | (SEQ ID NO: 32) | |
| EN1 | ACAGCAGCCGGAACCTAAAA | CCTTTTTGCAGCCGAAGTCC | 350 |
| | (SEQ ID NO: 33) | (SEQ ID NO: 34) | |
| VGLUT 1 | TCTCCTTCCTGGTCCTAGCC | TGCACCAGGGAGGCAATTAG | 223 |
| | (SEQ ID NO: 35) | (SEQ ID NO: 36) | |
| GABA | CGCTCAGTGGTTGTAGCAGA | AGCTGTTGCATAAGCCACCT | 327 |
| | (SEQ ID NO: 37) | (SEQ ID NO: 38) | |
| SYN | GCAGTTTGGTCATTGGGCTG | TTTGGCATCGATGAAGGGCT | 338 |
| | (SEQ ID NO: 39) | (SEQ ID NO: 40) | |
| Tuj 1 | CTGGCCATCCAGAGCAAGAA | CGTACATCTCGCCCTCTTCC | 322 |
| | (SEQ ID NO: 41) | (SEQ ID NO: 42) | |
| NF | CAGATCCAGTACGCGCAGAT | CGGCATGCTTCGATTTCCAG | 260 |
| | (SEQ ID NO: 43) | (SEQ ID NO: 44) | |
| MAP2 | GCACACTCACATCCACCTGA | CCTTGCAGACACCTCCTCTG | 210 |
| | (SEQ ID NO: 45) | (SEQ ID NO: 46) | |
| S100b | TGCAGCCTAGTAGGAGCTGA | CCTCCGGGTTAGGGTCTACA | 257 |
| | (SEQ ID NO: 47) | (SEQ ID NO: 48) | |
| MBP | GGATCACCCATGGCTAGACG | TCTGTCTCTGCAGCTGTGTG | 433 |
| | (SEQ ID NO: 49) | (SEQ ID NO: 50) | |
| CNPase | CTCTGAGACCCTCCGCAAAG | CTAAGAGGTCAAGGCCCGTC | 455 |
| | (SEQ ID NO: 51) | (SEQ ID NO: 52) | |
| ACTB | CACCACACCTTCTACAAT | TGATCTGGGTCATCTTCT | 109 |
| | (SEQ ID NO: 53) | (SEQ ID NO: 54) | |

### Gene Expression Microarray

Total RNAs of CB CD34+ cells, CB-iNSCs and hcx NSCs were extracted using the kits as mentioned above. RNA quantity and quality (2100 Bioanalyzer, Agilent Technologies) was determined to be optimal before further processing. The Affymetrix Human HG-U133plus2 GeneChip arrays hybridization, staining, and scanning, were performed using Affymetrix standard protocols (Affymetrix, Santa Clara, Calif.). All genes of neurogenesis and hematopoiesis according to Gene ontology (GO) terms (AmiGO, available online at the geneontology website) are analyzed and the upregulation or downregulation fold changes were normalized to CB CD34+ cells. The heat-map of gene expression levels was generated by R software.

Transplantation of CB-iNSCs into Mice

To track the CB-iNSCs in vivo, CB-iNSCs were labeled by transduction of GFP lentivirus before injection to the right striatum of NOD/SCID mice. Under anesthesia, the right sides of the mice skulls were exposed after tissue separation. A hole with a diameter of approximately 1.0 mm was drilled at center of the coordinates: AP: 0 mm; ML: 2.5 mm. Then, NOD/SCID mice were placed under stereotaxic apparatus and received 200,000 CB-iNSCs (in 2 μl PBS) at the coordinates: AP: 0 mm; ML: 2.5 mm; DV: 3.5 mm. The cell injection was finished in 5 minutes and the syringe was removed after another 5 minutes. Antibiotic (0.5% Bactrim in water) was given to the animals after surgery for two weeks. One month or three months after transplantation, the cryostat sections (10 μm) of animals' brain were prepared after intracardiac perfusion, fixation and dehydration. The density of migrated cells in contralateral hemisphere (left brain) were analyzed by counting GFP+ cells in six random fields of the coronal sections (AP: 0 mm) under 10× lens of the fluorescent microscope.

### Immunostaining

Cells were fixed in 4% paraformaldehyde for 15 minutes at room temperature and washed with PBS. Nonspecific antibody binding was blocked using 1% BSA for 30 minutes, and cells were permeabilized with 0.3% Triton X-100 (Sigma) in PBS (PBS-T) for 30 minutes at room temperature. Cells were rinsed and then incubated in primary antibody containing 0.1% overnight at 4° C. After washing in PBS, cells were incubated in secondary antibody 1 hour at room temperature. Cells were immunostained with the following anti-human primary antibodies: anti-Nestin, βIII tubulin (Tuj1), anti-MAP2 anti-glial fibrillary acidic protein (GFAP), and anti-MBP. Primary antibodies were detected with the PE conjugated secondary antibody. Stained cells with were preserved in anti-fading mount solution that contained DAPI. Stained cells were examined and photographed under EOVS fluorescent microscope.

### Electrophysiology

Glass coverslips containing differentiated cells derived from CD34+-iNSC cells were transferred to a Zeiss microscope with DIC and phase-contrast optics. In the whole-cell patch clamp, cells with a relatively large cell body and neurite like structures were chosen for recording. Cells were perfused with a standard bathing medium (140 mM NaCl, 5 mM KCl, 1.5 mM CaCl2, 1 mM MgCl2, 10 mM HEPES, pH 7.2, 37 1 C). Electrodes were pulled from borosilicate glass and filled with intracellular recording solution (100 mM KCH3SO3, 40 mM KCl, 0.2 mM EGTA, 0.02 mM CaCl2, 1 mM MgCl2, 2 mM ATP, 300 mM GTP, 10 mM HEPES buffer) for voltage clamp measures. Potassium currents were elicited by step depolarization of the membrane in 20-mV increments from −110 to +110 mV.

### Results

Reprogramming of Cord Blood CD34⁺ Cells into Hemangioblast-Like Cells Using a Feeder-Free System

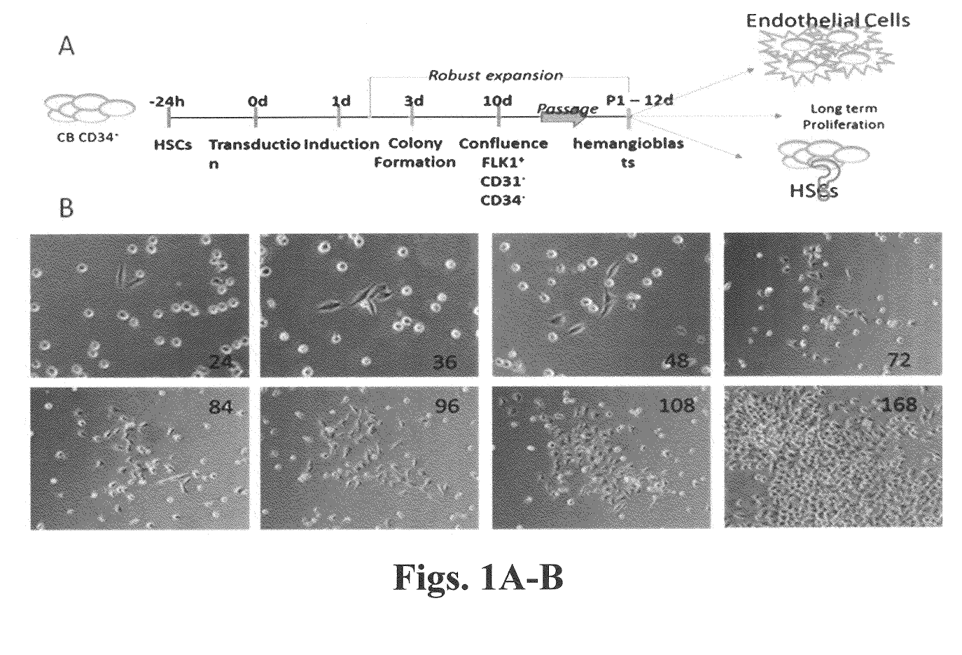

Hemangioblast-like cells, termed Multi-Potent Stem Cells (MPSCs) hereafter, were derived according to the timeline shown in FIG. 1A. We transduced cord blood CD34⁺ cells with lentivirus containing OCT4 or control GFP gene under the SFFV promoter. Adherent cells with a round and spindle-like morphology appeared as early as 24 hours (FIG. 1B). These adherent cells exhibit a high proliferative potential expanding at a rapid doubling rate of 20 hours and developed into colony like groups that possessed similar morphology to hemangioblasts (Kennedy et al, Nature, 1997, 383:488-493). In contrast, we never obtained such colonies from cord blood cells transduced with control GFP vector. Although some adherent cells were observed, none had the proliferative potential of those transduced with OCT4, which typically reached confluence around 10-12 days after transduction and continue to grow at a doubling rate of 14-16 hours.

### Characterization of Hemangioblast-Like Cells

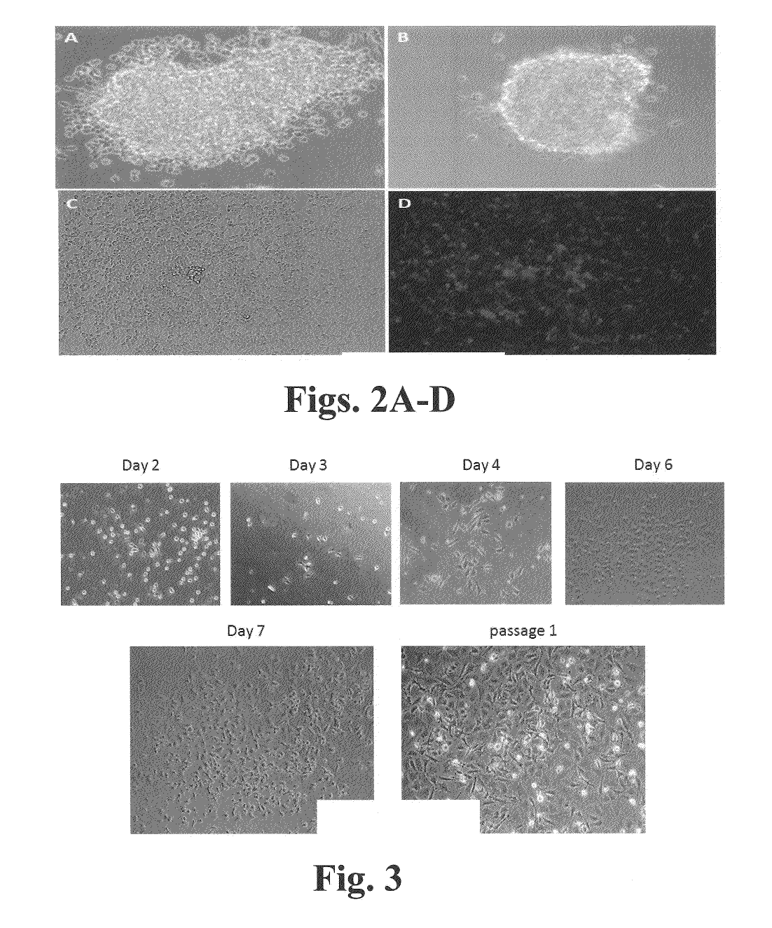

One of the defining characteristics of hemangioblasts is their blast-like colony forming cells (BL-CFCs) (Kennedy et al, Nature, 1997, 383:488-493). In the absence of ECM proteins, we observed the formation of these blast-like colonies within hours of plating and continued growth in that state for weeks (FIGS. 2A and 2B). Upon plating onto ECM treated plates, they were able to re-attach and continue to proliferate.

We found immunofluorescence of the developing colonies on Day 4, 8 and 12 after transduction with OCT4. The colonies were found to be negative for CD31 and CD34 and positive for FLK1 as early as day 4. FLK1 signal appeared weak initially, but significantly increased in intensity and quantity, reaching approximately 95% by day 8. These cells continue to maintain their FLK1⁺ signals for 4 passages without any loss, after which they begin to slowly lose their signal reaching approximately 50% by passage 8. In addition, the cells beginning on Day 4 are negative for CD31 and CD34 and remain thus throughout culture.

### Generation of Hemangioblast-Like Cells Via Non-Integrating System

Using an episome vector (pCEB) containing SFFV-OCT4, we were able to generate hemangioblast-like cells with almost identical morphology under hypoxic conditions (FIG. 3) using a non-integrating DNA system. Cord blood CD34⁺ cells were electroporated with our episome construct and the appearance of attachment cells appeared in roughly the same timeframe as the lentiviral counterparts with a delay of 1-2 days. We observed similar morphological behaviors and even similar differentiation capabilities as the lentiviral counterparts. These episome-derived cells began to lose their OCT4 expression around day 21 and lost OCT4 expression almost entirely by 28 days after transduction.

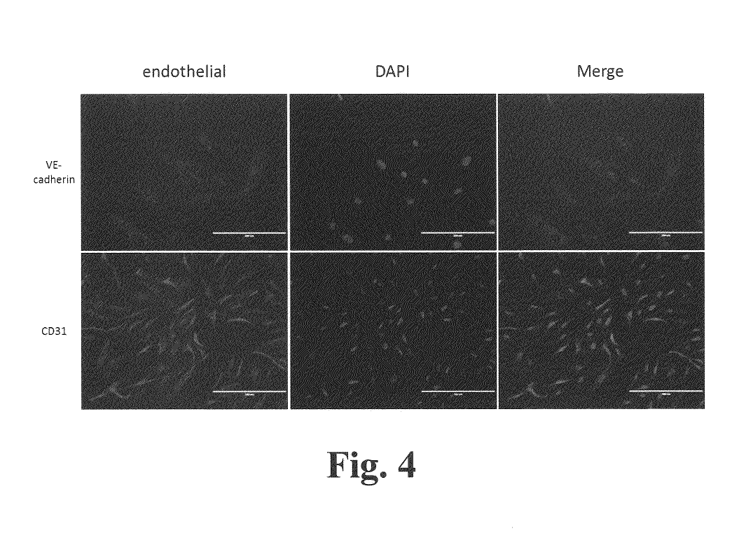

One of the defining characteristics of hemangioblasts/MPSCs is their ability to differentiate into endothelial and hematopoietic stem cells. We induced endothelial cell differentiation by culturing MPSCs with an endothelial cell growth medium supplemented with TGF-13 inhibitor and 8-Br-cAMP to push the cells toward endothelial cell differentiation. Cells began to look morphologically different after 2 days of culture, becoming larger in size and resembling that of HUVEC cells. Through immunofluorescence, we observed CD31 and VE-Cadherin expression as early as day 6 into culture (FIG. 4).

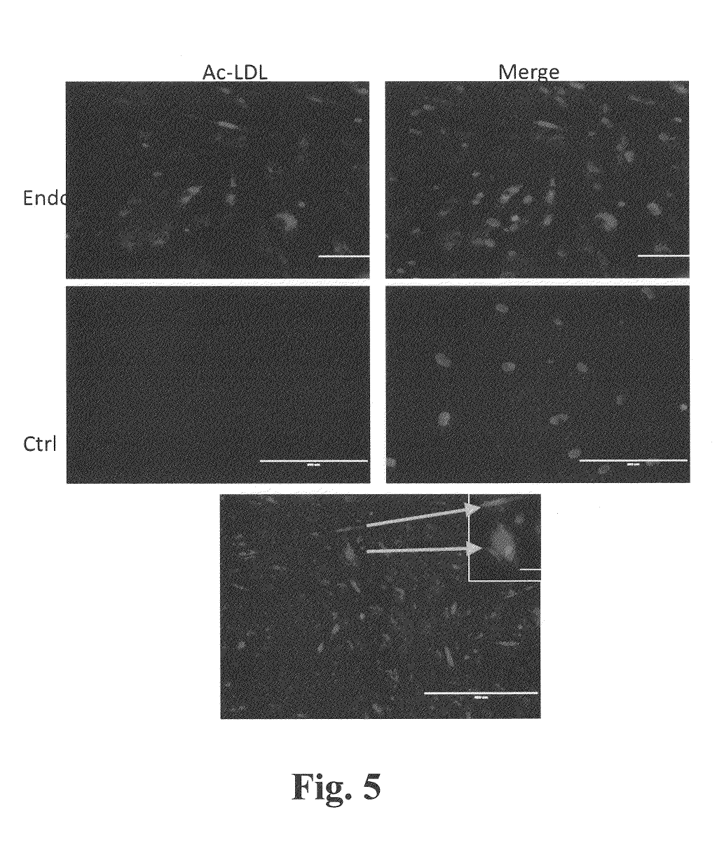

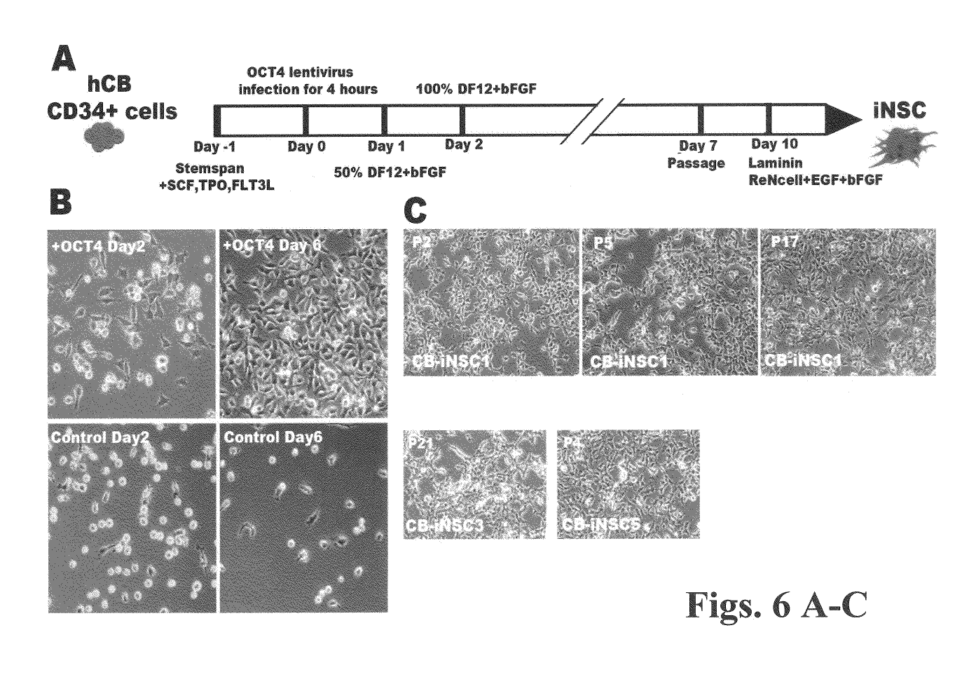

We further characterized our endothelial cells by testing the “scavenger-cell pathway” to observe the Ac-LDL uptake that is limited to only endothelial cells and macrophages. We observed an 80% uptake of Ac-LDL by all the cells, 10-15% of which were observed to absorb a significantly greater amount (FIG. 5). The control cells or undifferentiated MPSCs did not show any uptake in Ac-LDL.

### Differentiation of Hemangioblast Like-Cells to Hematopoietic Cells

To further characterize hemangioblast-like cells dedifferentiated from CD34+ cells, we differentiated this population to hematopoietic cells. The hemangioblast-like cells were cultured with bone morphogenetic protein 4 (BMP-4) and minimal cytokines (SCF, TPO, Flit3) for four days. When BMP-4 was removed for 6 days, numerous clusters of hematopoietic-like cells in structures similar in appearance to embryonic blood islands were present. The cultures were first analyzed in situ for a key marker, CD45, with the live cell stain using a fluorescent CD45 antibody, and blood island clusters were confirmed to be positive for CD45. We then isolated and expanded these clusters. Live cell stains with CD45 and CD34 antibodies demonstrated that they were CD45 and CD34 positive, indicating hematopoietic stem/progenitor cells.

Plasticity of the Hemangioblast Like Cells—Differentiation into Neural Stem Cells

Hemangioblast-like cells/MPSCs were capable of differentiating into neural progenitors demonstrating plasticity in its capability to cross from mesoderm state into the ectoderm lineage. We initiated the differentiation by plating the freshly passaged cells directly on laminin-treated tissue culture plates in ReNcell medium in the presence of human EGF and bFGF, a well-established culture condition for human neural progenitor cells. The cells, which we will term induced neural stem cells (iNSCs) hereafter, were found to express neural precursor markers such as Nestin and Musashi-1 (MSI-1) which were absent in the MPSCs. The gene expression patterns by real-time PCR found Nestin, Musashi −1 and Pax6 were significantly upregulated compared to control CB cells, while hematological markers CD34, CD38 and CD45 were dramatically downregulated.

When the growth factors were removed from the basal ReNcell medium, the growth speed of the iNSCs significantly slowed down as more and longer neuritis appeared, an indication of the differentiation of these neural progenitors into the three lineages of neural cells: oligodendrocytes, astrocytes and neurons. By immunofluorescence, we found these iNSCs were capable of differentiating into these three lineages as characterized by their positive markers for GFAP (an astrocyte marker), MBP (an oligodendrocyte marker), and Tuj1 (a neuron marker). Additionally, iNSCs were able to generate functional neurons as characterized by their electrophysiology.

Reprogramming of Cord Blood CD34+ Cells into Neural Stem Cells in a Feeder-Free System

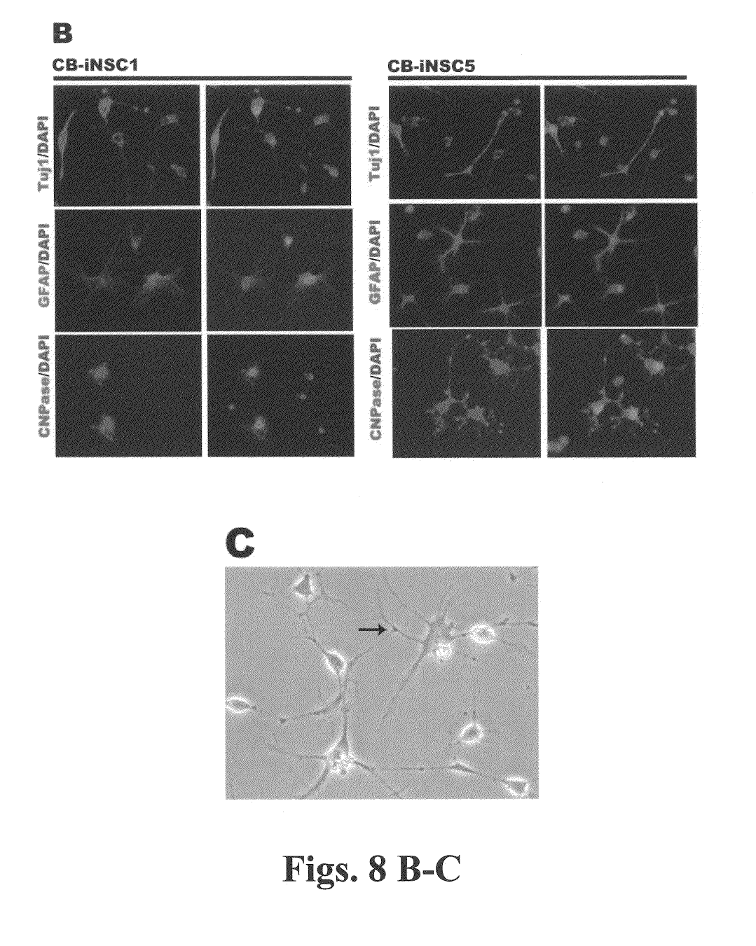

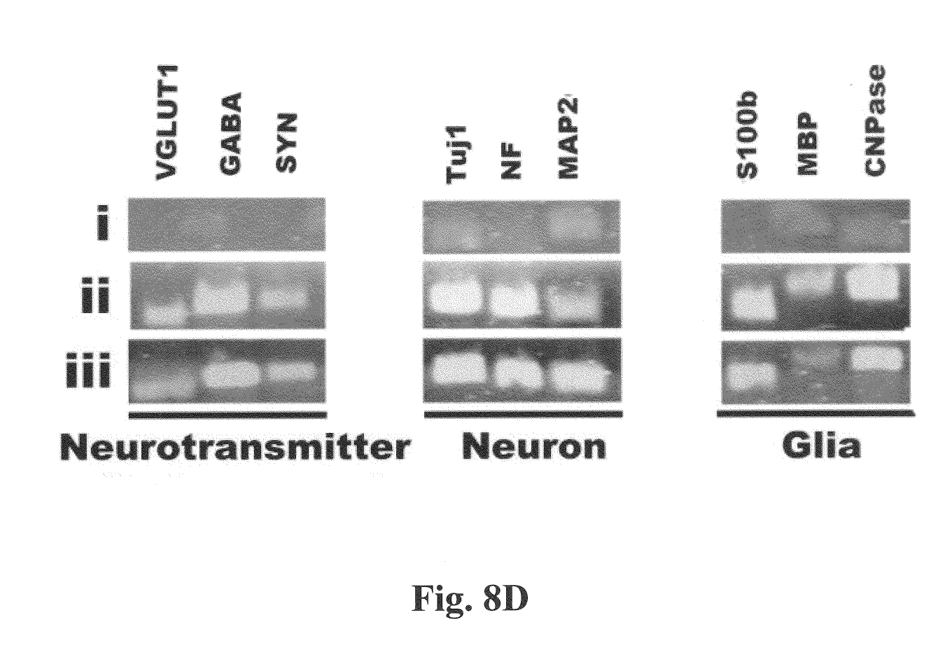

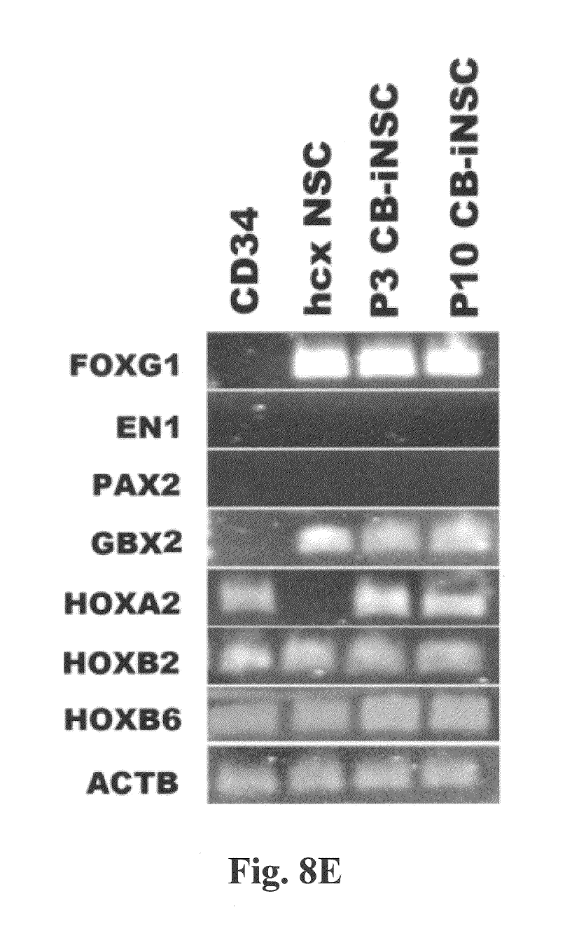

We then transduced cord blood CD34+ with lentiviruses carrying OCT4 or control GFP gene under the SFFV promoter (FIG. 8A). Adherent spindle-like cells could be observed as early as 24 hours after SFFV-Oct4 transduction in human cord blood CD34+ cells, and these adherent cells exhibited high proliferation potency in the medium containing human bFGF supplement (FIG. 8B). In contrast, we never obtained proliferative adherent cells in cord blood cells with control SFFV-GFP vector, although some adherent cells could be detected in the culture, nor did we get proliferative cells from CMV-Oct4 transduced cord blood cells. Additional attempts to generate proliferative adherent cells from cord blood CD34− cells were not successful. Routinely in our system, the adherent cells in Oct4 groups reach confluence 7 to 9 days after transduction. These cells were dissociated into single cells and cultured in NSC medium. The cells showed obvious morphological change in NSC medium; they became more uniform with higher light reflection of the cell bodies. More importantly, these cord blood cell induced NSCs (CB-iNSCs), were highly expandable (>20 passages, >2 months in culture) and maintained the morphology and doubling times (24-36 hours) in vitro (FIG. 8 C).

In order to detect if there are any neural precursor cells in the fraction of cord blood CD34+ cells, we plated CD34+ cells directly on laminin-coated tissue culture plates in ReNcell medium in the presence of human EGF and bFGF. As a control, human Cortex Neural Stem Cells (hcx NSC) were cultured under the same conditions. We did not obtain any adherent cells resembling NSC from non-transduced cells. In addition, we failed to detect any expression of neural stem cell markers in the cord blood cells. These results ruled out the possible existence of neural precursor cells in the original CD34+ cell population.

### Characterization of CB-iNSCs

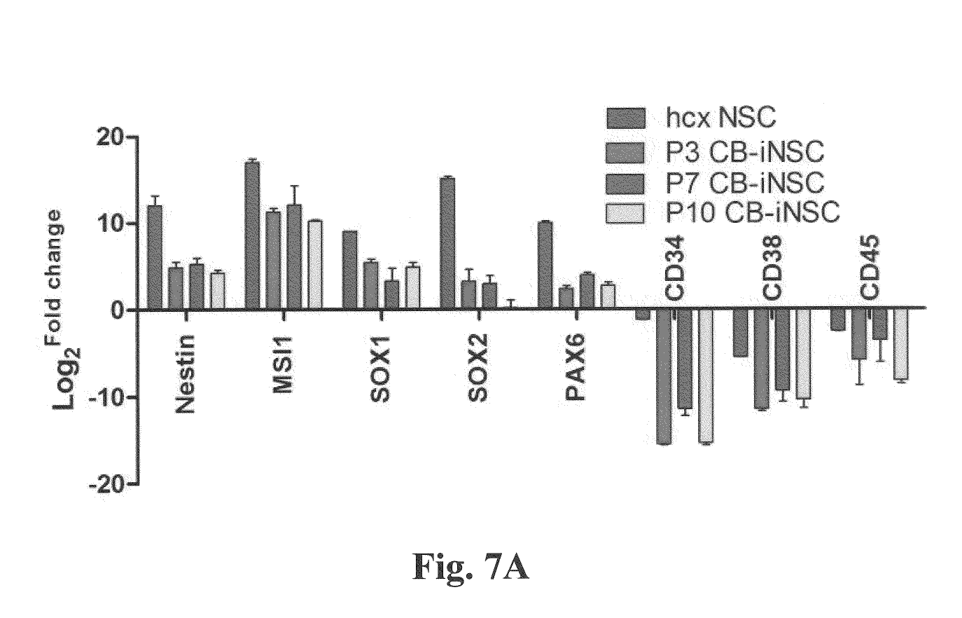

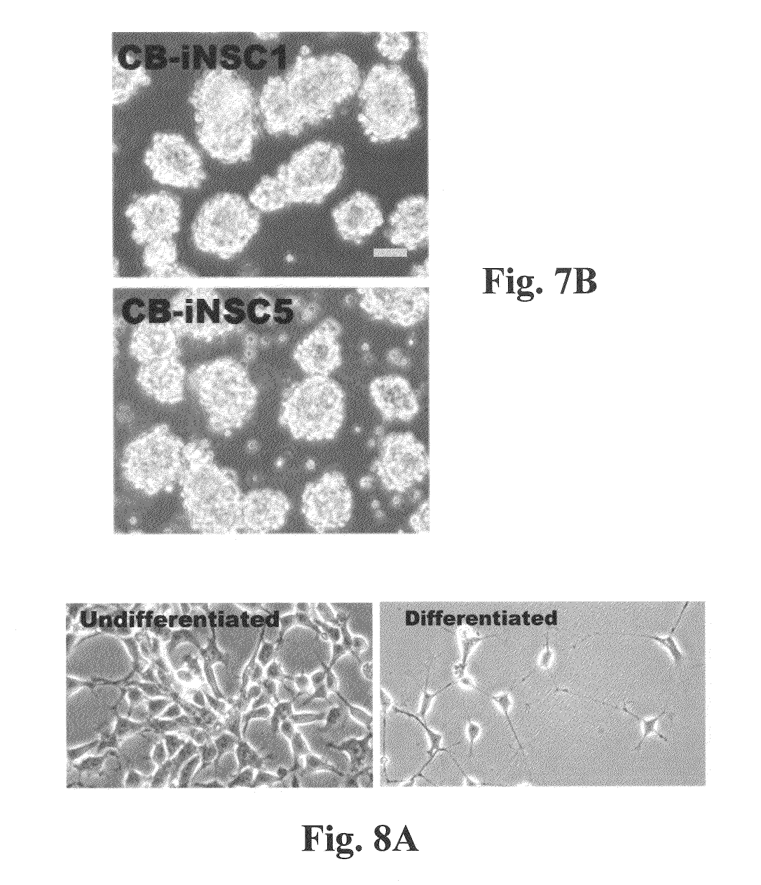

In addition to the typical NSC-like morphology of CB-iNSCs, by immunostaining, we found that they expressed neural precursor markers such as Nestin, Musashi-1 (MSI-1) and Pax6, at comparable levels to expression of these markers in hcx NSCs. We also analyzed the gene expression patterns of CB-iNSCs by real-time PCR (FIG. 7A). Compared to the original cord blood cells, the neural precursor markers Nestin, Mushasi-1 (MSI-1), Sox1 and Pax6 were significantly upregulated, while the hematological markers CD34, CD38 and CD45 were dramatically downregulated. The gene expression pattern of neural stem cells and hematopoietic markers is similar in CB-iNSCs and hcx NSCs as compared to CB cells. Of note is that the overall gene expression levels were elevated in hcx NSCs with regard to both neural stem cells and hematopoietic markers. This may be due to the ectopic expression of the oncogene C-myc in hcx NSCs line. Further analysis using gene expression arrays confirm that CB-iNSCs are similar to hcx NSC cells in gene expression patterns in neurogenesis and hematopoiesis (data not shown). We also compared the gene expression pattern of early and late passages CB-iNSCs and found no obvious differences between the results of passage 3 and passage 10 CB-iNSCs, suggesting they a maintained stem cell signature during prolonged in vitro culture. An interesting observation is that Sox2 expression is only slightly elevated in CB-iNSCs compared to CB CD34+ cells. Additionally, CB-iNSCs could form neurospheres in low-adherent culture dishes (FIG. 7B). Therefore, CB-iNSCs have the characteristics of neural precursor cells not seen in CB originating cells.

### Regional Pattern of CB-iNSCs

In the development of the neural system, there are distinct expressions of transcriptional factors in the precursors for forebrain, midbrain and hindbrain regions. We detected forebrain (FOXG1), hindbrain (HOXA2, GBX1, EGR2 and HOXB2) and also spinal cord (HoxB6) markers, but not midbrain markers (PAX2 and EN1) in our CB-iNSCs (FIG. 8E), and the forebrain and hindbrain regional specificities did not change in the late passage as we see the same expression in Passage 3 and Passage 10 CB-iNSCs. The control hcx NSCs also showed positive expression of forebrain (FOXG1) and hindbrain (GBX1, EGR2 and HOXB2) markers, and negative for midbrain markers (PAX2 and EN1). The original CB CD34+ cells are negative for all of the midbrain and forebrain markers we tested, and also negative for hindbrain GBX1.

### Differentiation of CB-iNSCs In Vitro

When the growth factors EGF and bFGF were removed from the ReNcell medium, the growth of CB-iNSCs largely slowed down and went to differentiation, an indication of which is that more and longer neurites developed from the cells as the culture continued (FIG. 8A). Synapse-like structures between cells could be seen after 2 weeks in vitro (FIG. 8B-C), consistent with the positive synapsin1 expression detected by PCR assay. By immunostaining, we found some of the cells expressed Tuj1, MAP2, GFAP or MBP, indicating the multi-lineage differentiation capacity of CB-iNSCs (FIG. 8C). This was confirmed by PCR test results (FIG. 8D) in which markers of all three lineages of neural cells were detected: Tuj1, MAP2, Neurofilament (NF) for neurons; S100B and MBP (Myelin basic protein) for astrocytes; and CNPase for oligodendrocytes. In addition, we saw expression of both vesicular glutamate transporter 1 (VGLUT1) and γ-aminobutyric acid (GABA) receptor in the differentiated cells, suggesting both excitatory (glutamatergic) and inhibitory (GABAergic) neurons were able to be generated from CB-iNSCs (FIG. 8D). In addition, electrophysiological assays showed that the differentiated neural cells exhibited dramatic response in current changes as evoked by voltage steps in whole-cell patch-clamp. In contrast, the undifferentiated CB-iNSCs only had slight response in the assay.

### CB-iNSCs Engraftment in Mouse Brain

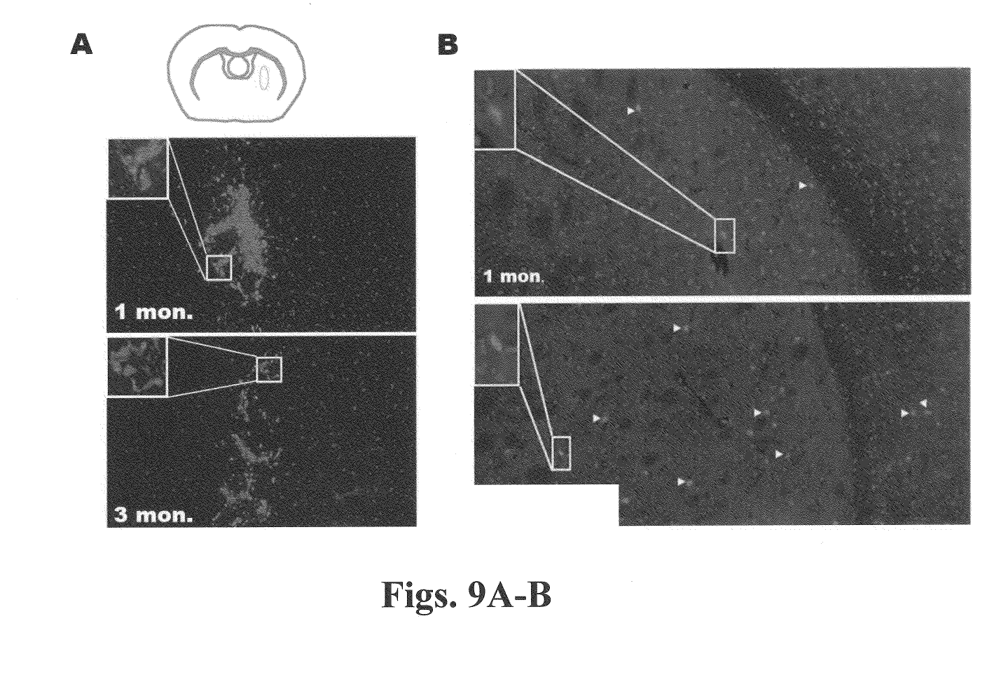

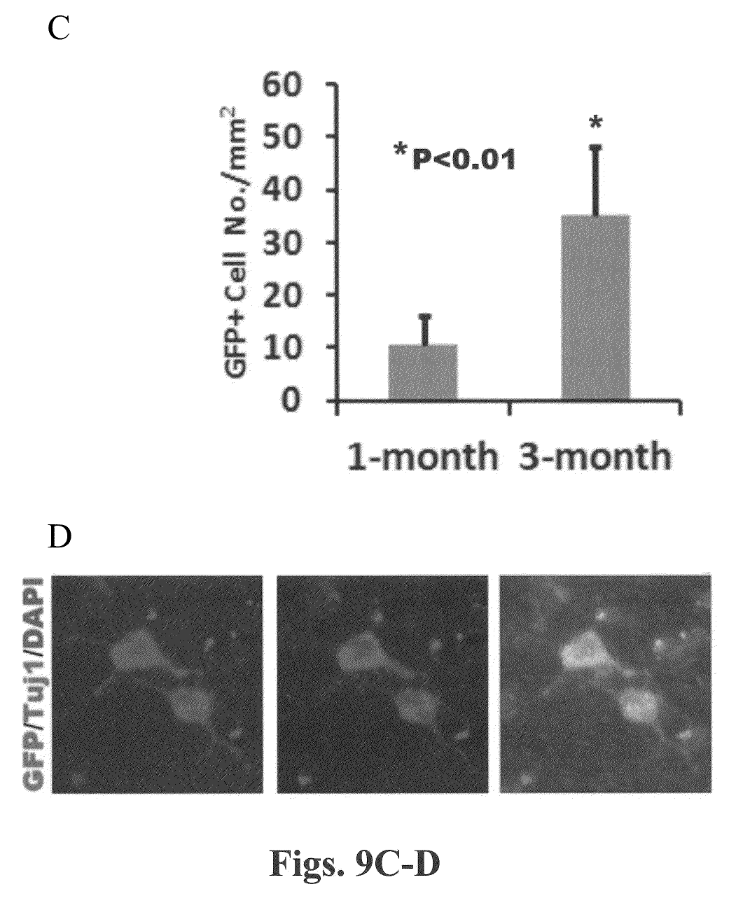

We labeled the CB-iNSCs with GFP marker and injected them into NOD/SCID mouse striatum (FIGS. 9A-B). We found a large proportion of these cells still survived in the brain one month and three months after transplant (FIG. 9C). Long neurites could be detected from some of the cells. By immunostaining, we found Tuj1 or GFAP expressing cells from the GFP positive injected cells (FIG. 9D), suggesting the maturation of CB-iNSCs. We also monitored some of the mice after receiving CB-iNSCs for long term safety. There was no abnormality observed more than 3 months after injection.

### Generation of Integration-Free CB-iNSCs

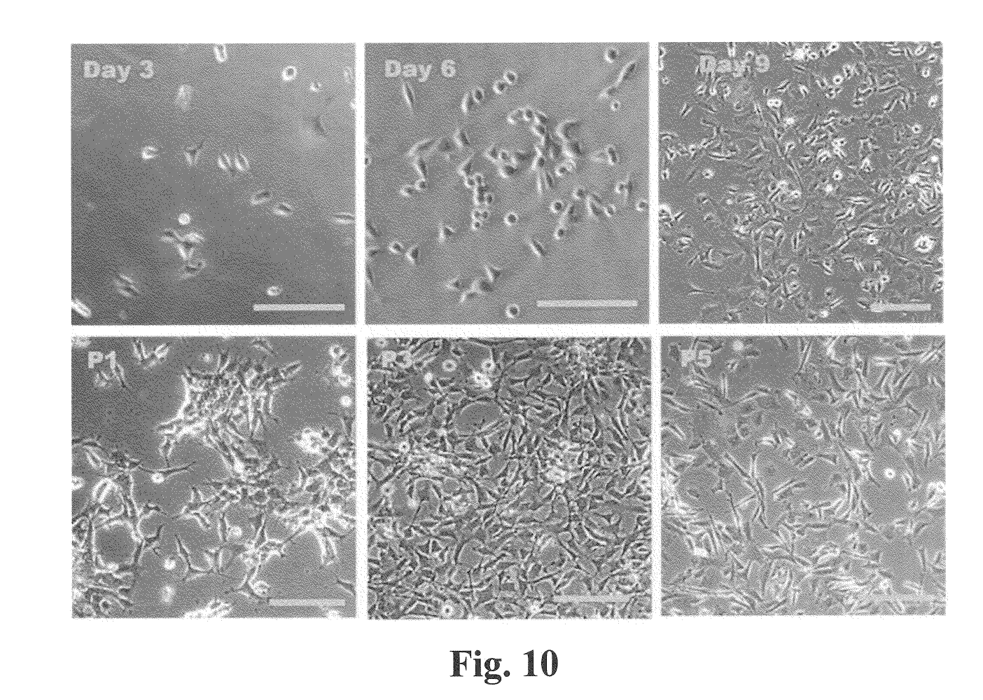

While direct reprogramming using lentivirus does provide an increasingly popular approach that bypasses the iPS cell state, the issue of random integration of foreign DNA into the host's genome still remains. We attempted to generate iNSCs from CB by an integration-free method. CB CD34⁺ were electroporated with an episomal vector containing a high expression OCT4 gene. After nucleofection, we successfully generated neural stem cells within 2-3 weeks (FIG. 10), which we hereafter term as eNSCs for brevity. iNSCs and eiNSCs are identical morphologically, and eiNSCs did not differ in the expression of typical NSC markers of Nestin and Musashi. In addition, eiNSCs are able to differentiate into neural and glial cells as neuron and astrocyte markers were observed after random differentiation. However, eiNSCs are slower in growth rate with a doubling time of approximately 36 hours where iNSCs have a doubling time of approximately 24 hours. Also, eiNSCs failed to maintain long-term expansion in vitro as no growth was observed after three to four passages. When Dzep, a compound that was recently reported to increase the efficiency of iPS generation was added to the culture, eiNSCs grew for two more passages.

### Reprogramming of Pancreatic Islet Beta Cells

The experimental studies described below show the ability of SFFV or EF1-OCT4 transduction to drive insulin-producing β-cells (beta cells) to return to more primitive developmental state, stem-like cells by a high level of OCT4 expression. The embryonic state is “skipped”. The process allows generation of pancreatic beta cells on a large scale for therapy.

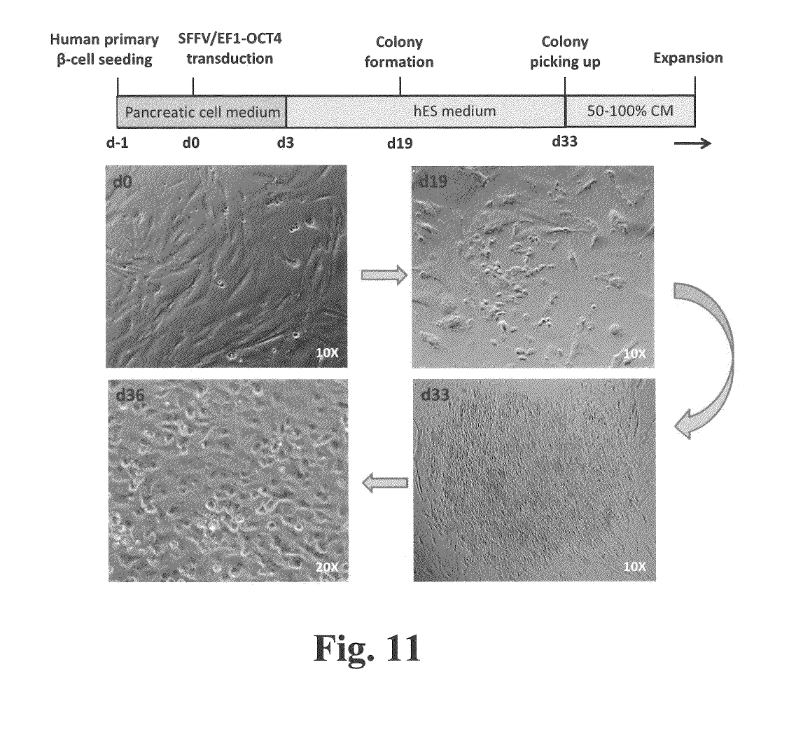

Beta cells were transduced with SFFV or EF1-OCT4 and seeded in multi-well plates at 5×10³ beta cells per well. About 20 small reprogrammed colonies were observed in each well at about day 17 to 19 after SFFV or EF1-OCT4 transduction (FIG. 11). By day 28-30 after transduction, a specific surface marker for PDX1-positive pancreatic progenitors, CD24, became positive. These cells were also negative for specific markers for ES cells (TRA-1-60). By day 30-33 post transduction cells could be selected for further differentiation expansion.

### Dedifferentiating or Reprogramming of Fresh Isolated Human Islet Cells to Stem-Like Cells

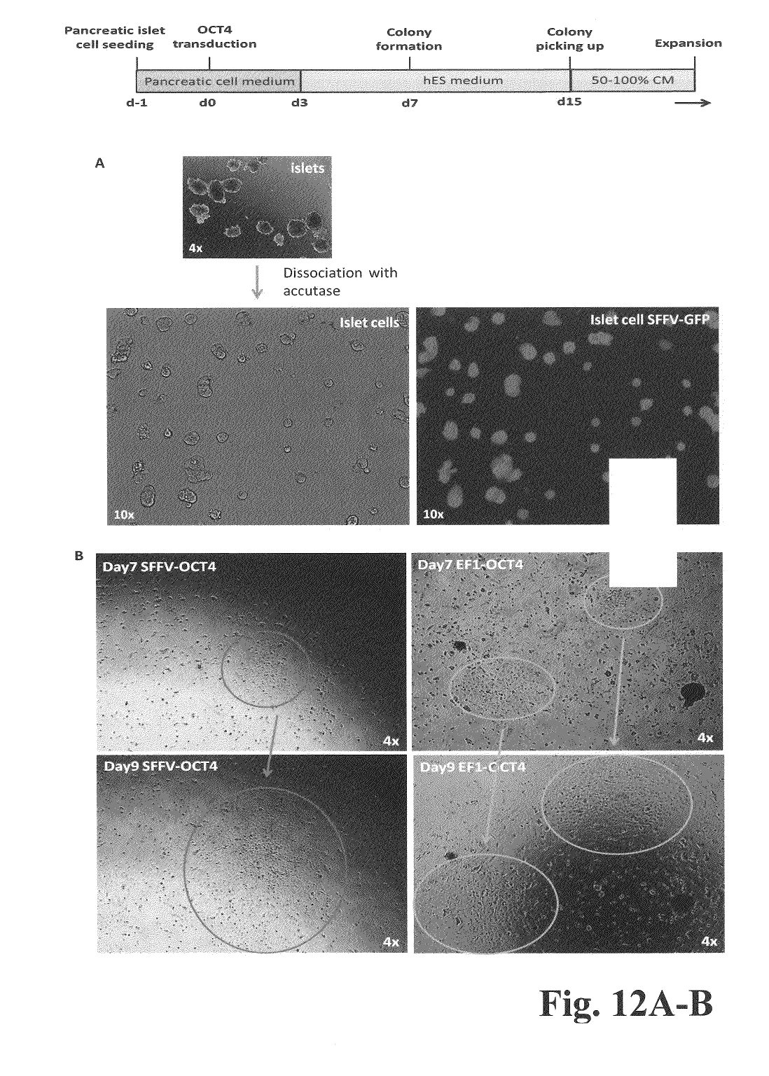

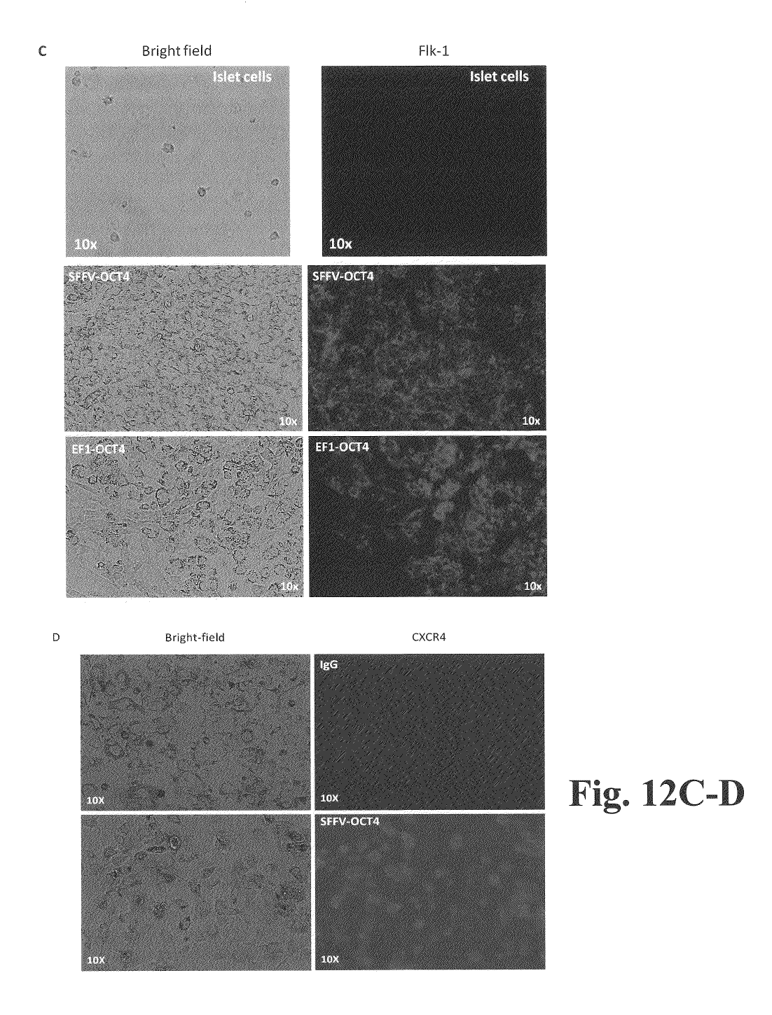

Fresh human pancreatic islet cells were obtained (Prodo Laoratories LLC) and dissociated (FIG. 12A). The dissociated islet cells, 1×10³ were then transduced with either SFFV-OCT4 or EF1-OCT4 lentiviruses. Approximately 20 colonies were observed after 7 day viral transduction (FIG. 12B). By day 10, all colonies became strongly positive for FLK1. We also examined the endoderm marker CXCR4 (Thermo Fisher Scientific Inc.), and FLK1 positive colonies were also positive for CXCR4 indicative of endoderm derivatives.

### Small Molecules Related to Stem Cell Expansion and Reprogramming

Recent studies show that, in a mouse model, pluripotent stem cells can be generated from mouse somatic cells using a combination of six to seven small-molecule compounds (Hou et al, Science, 341(6146):651-654). These small molecules include: valproic acid (“V”); CHIR99021 (“C”), a glycogen synthase kinase 3 inhibitor; 616452, a transforming growth factor-beta receptor1 kinase inhibitor II (“6”); tranylcypromine hydrochloride (“T”), an inhibitor of the histone lysine demethylase LSD1; forskolin/FSK (“F”), a cAMP agonist; and DZNep/3-deazaneplanocin A (“Z”), a lysine methyltransferase EZH2 (KMT6) inhibitor. These compounds are collectively referred to as “VC6TFZ”. However, it remains to be determined if these compounds are able to function in a similar fashion in humans. We now identify that some of molecules are able to expand stem cells or maintain stem cell properties. These molecules, in combinations we identified, can reprogram human somatic cells.

Valproic acid (“V”) is sodium 2-propylpentanoate, with the structure:

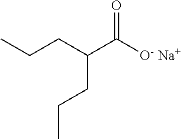

CHIR99021 (“C”) is 6-[[2-[[4-(2,4-dichlorophenyl)-5-(5-methyl-1H-imidazol-2-yl)-2 pyrimidinyl]amino]ethyl]amino]-3-pyridinecarbonitrile, with the structure:

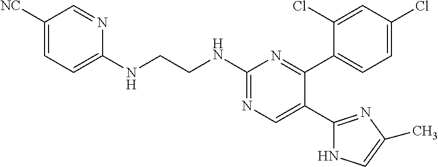 [SMILES: Cc1c[nH]c(-c2cnc(NCCNc3ccc(C#N)cn3)nc2-c2ccc(Cl)cc2Cl)n1]

616452 (“6”) is 2-[3-(6-methyl-2-pyridinyl)-1H-pyrazol-4-yl]-1,5-naphthyridine, with the structure:

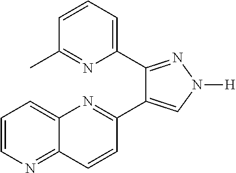 [SMILES: Cc1cccc(-c2n[nH]cc2-c2ccc3ncccc3n2)n1]

Tranylcypromine hydrochloride (“T”) is (±)-trans-2-Phenylcyclopropylamine hydrochloride, with the structure:

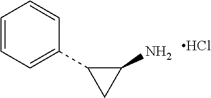

Forskolin/FSK (“F”) is 7β-acetoxy-8,13-epoxy-1α,6β,9α-trihydroxylabd-14-en-11-one, with the structure:

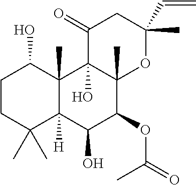

3-deazaneplanocin A/DZNep (“Z”) is 5R-(4-amino-1H-imidazo[4,5-c]pyridin-1-yl)-3-(hydroxymethyl)-3-cyclopentene-1S,2R-diol, with the structure:

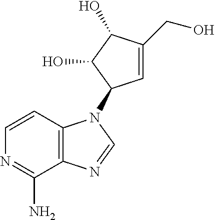 [SMILES: Nc1nccc2c1ncn2[C@@H]1C=C(CO)[C@@H](O)[C@H]1O]

616454 (“4”) is 3-(6-Methylpyridin-2-yl)-4-(4-quinolyl)-1-phenylthiocarbamoyl-1H-pyrazole, with the structure:

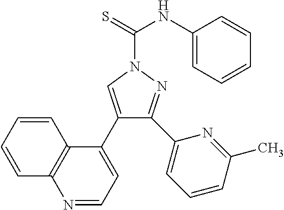 [SMILES: Cc1cccc(-c2nn(C(=S)Nc3ccccc3)cc2-c2ccnc3ccccc23)n1]

SB 431452 (“5”) is 4-[4-(1,3-benzodioxol-5-yl)-5-(2-pyridinyl)-1H-imidazol-2-yl]benzamide, with the structure:

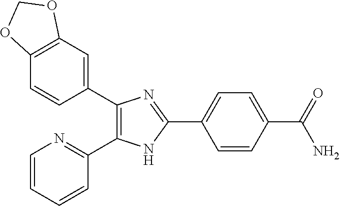 [SMILES: NC(=O)c1ccc(-c2nc(-c3ccc4c(c3)OCO4)c(-c3ccccn3)[nH]2)cc1]

CD34+ cells isolated from peripheral blood of G-CSF-mobilized donors were cultured for 11 days with minimal cytokines and the 4 chemical cocktail (C6FZ). There was a significant impact and enhancement of the HSPC (hematopoietic stem/progenitor cells) population on ex vivo cultures using C6FZ. Within 11 days of culture, we observed that there was a significant increase the proportion of population with a marker, CD34⁺/CD38⁻ (39% in 4 compounds treated vs 0.8% in the minimal cytokines alone) by flow cytometry analysis. The population bearing CD34+/CD38− is associated with long-term repopulating HSCs. However, this came at the cost of significant reduced cell growth. By day 5, we noticed an 18-fold growth in the control cells compared to the 5-fold growth of the cells induced with the 4 chemicals. The slow growth in the treated cells was further tested with the removal of single chemicals from the cocktail.

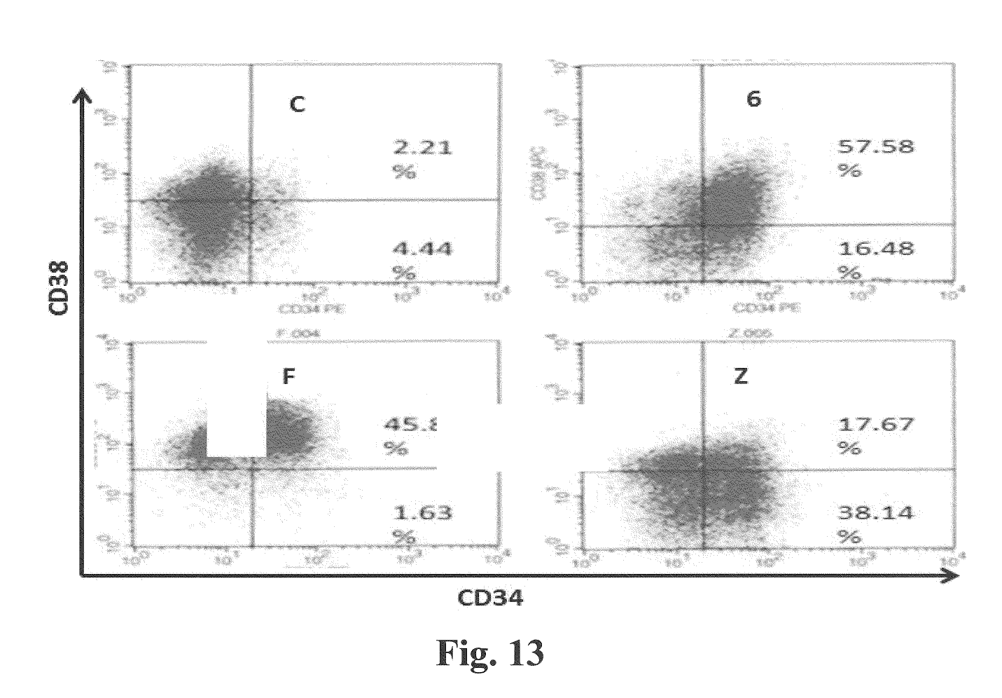

CD34+ cells isolated from the peripheral blood of G-CSF-mobilized donors were next cultured for 11 days with minimal cytokines and the individual compounds, C, 6, F and Z (FIG. 13). Chemical C/CHIR99021 alone rapidly proliferated the bone marrow CD34+ cells, leading to their maturation at a significantly rapid rate compared to control, but did not have much effect on either CD34+CD38+ or CD34+CD38− populations. Chemical F appeared to be able to induce a significant proportion of CD34+CD38+ cells while chemical 6 and Z each individually showed ability to enhance both the population of single positive (CD34+CD38−) and double positive (CD34+CD38+) cells.

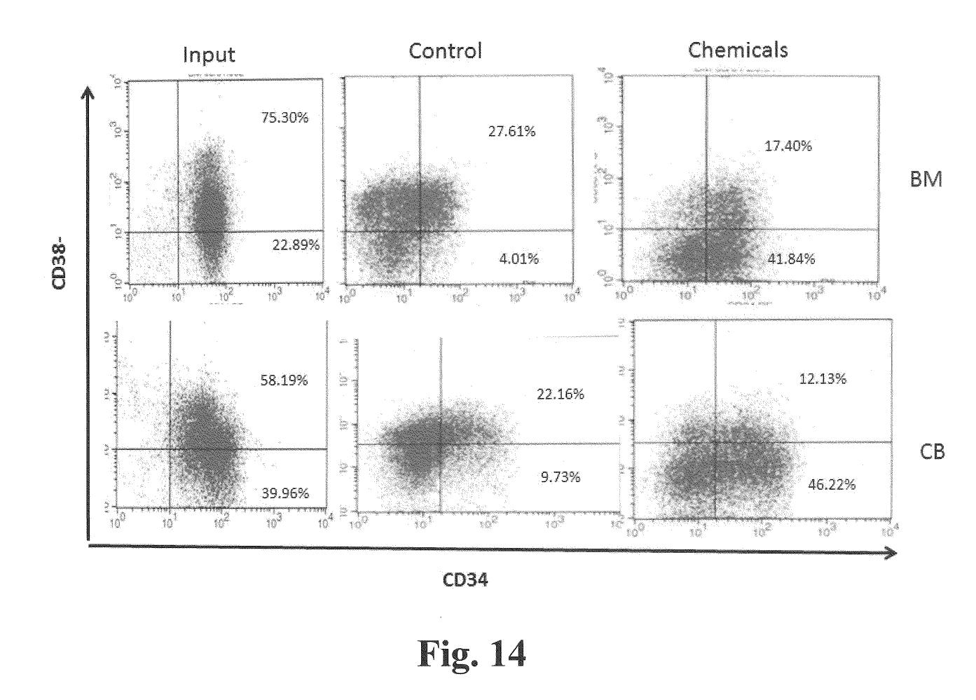

Further analysis of a combination of chemical compounds 6 and Z was performed on the CD34+ cells isolated from bone marrow. After a 6 day culture under chemicals 6Z, the population of CD34+CD38− rose to approximately 28-fold of the input of this cell type, compared to a 10-fold increase in the untreated cells. In addition, 6Z-treated cells better retained CD34+CD38− markers (˜41% in the treated cells vs ˜4% in the untreated cells) relative to untreated and input cells (FIG. 14). A similar observation was seen when chemicals 6 and Z were used to treat human umbilical cord blood cells. Culture of human CB CD34+ cells for 6 days with chemicals 6 and Z resulted in a 28-fold increase in CD34+CD38− cells compared with input cells while this population seen in untreated cells was only increased by 16 fold.

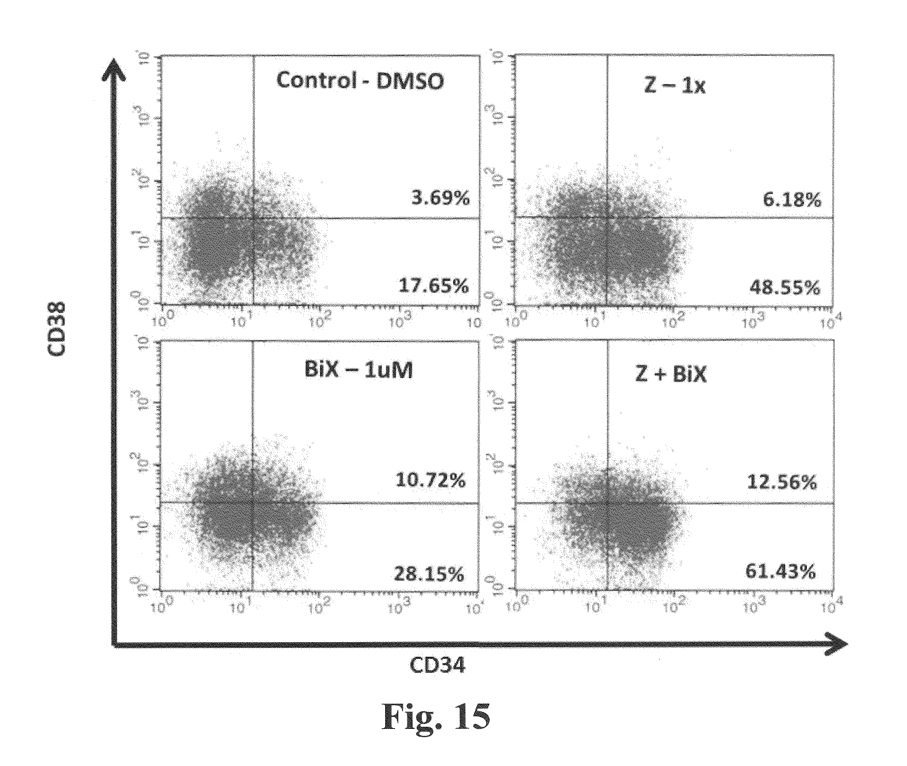

Recent studies in iPS cell reprogramming have discovered numerous small molecules modulating iPS cell reprogramming through different cellular mechanisms. These could include cellular signaling pathways (such as Wnt signaling and TGF beta) and epigenetic mechanisms, such as DNA methyltransferases, histone methylation and histone deacetylation. We have screened small molecule modulators involving these cellular mechanisms for hematopoietic stem/progenitor cell expansion. Only a very small subset of modulators appeared to expand or retain a significant proportion of CD34+CD38− population ex vivo (FIG. 13), which is associated with long-term engraftment of bone marrow stem cell transplantation. CB CD34+ cells were cultured for 5 days in HSC media containing SCF, TPO, and Flt-3 ligand at 100 ng/ml each. As shown in FIG. 15, cells were analyzed by flow cytometry on Day 6. Z at 1× (100 nM) or Bix at 1 μM retained a large portion of CD34+CD38− stem/progenitor cells as compared to control. When combined both together, there was a synergistic effect on retaining a significant proportion of CD34+CD38− population of nearly 62% after 5 day culture. Bix (BIX01294), trihydrochloride hydrate is a histone methyltransferase (HMTase) G9a inhibitor (FIG. 15).

Bix 01294 (“Bix”) is 2-(Hexahydro-4-methyl-1H-1,4-diazepin-1-yl)-6,7-dimethoxy-N-[1-(phenylmethyl)-4-piperidinyl]-4-quinazolinamine trihydrochloride hydrate, with the structure:

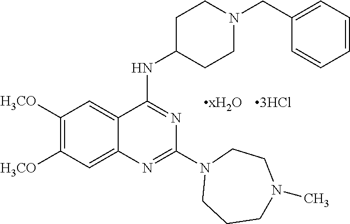 [SMILES: COc1cc2nc(N3CCCN(C)CC3)nc(NC3CCN(Cc4ccccc4)CC3)c2cc1OC]

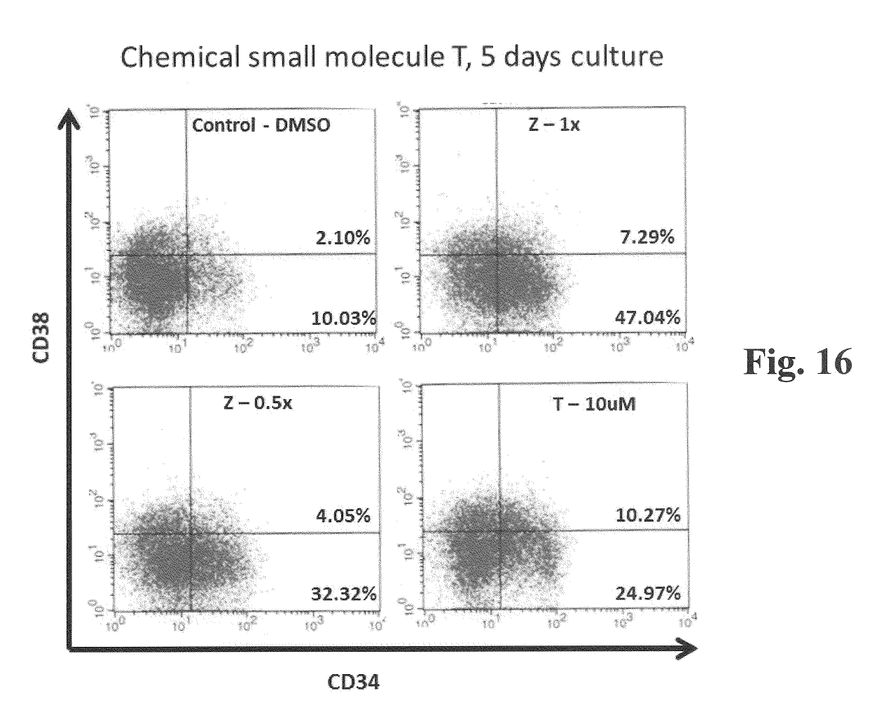

Previously we have shown that stem cell protein is a robust stimulator for hematopoietic stem/progenitor cell expansion via recruitment of various epigenetic factors such as histone deacylases and DNA methyltransferases (Yang et al, J Biol Chem. 2012, 13; 287(3): 1996-2005). We have also demonstrated that LSD1, a histone lysine demethylase plays an important role in the repressive effects of SALL4 on the hematopoiesis (Liu et al, J Bio Chem, 288:34719-28). Reduction of LSD1 in hematopoietic precursor cells by shRNA-mediated knockdown results in the expansion of this population. Based on this finding, we screened the LSD1 inhibitors for the expansion of hematopoietic precursor cells. hUCB CD34+ cells were cultured for 5 days in HSC media containing SCF, TPO, and Flt-3 ligand at 100 ng/ml each. As seen in FIG. 16, tranylcypromine hydrochloride (T), LSD1 inhibitor V at 10 μM retained a significant fraction of CD34+CD38− cells and expanded this population, as compared to that of the control. When compared to the input, T small molecule treatment resulted in an approximately 60- and 45-fold increase in CD34+CD38− cells and total cell counts, respectively, after 5 day culture.

### Megakaryocyte Differentiation

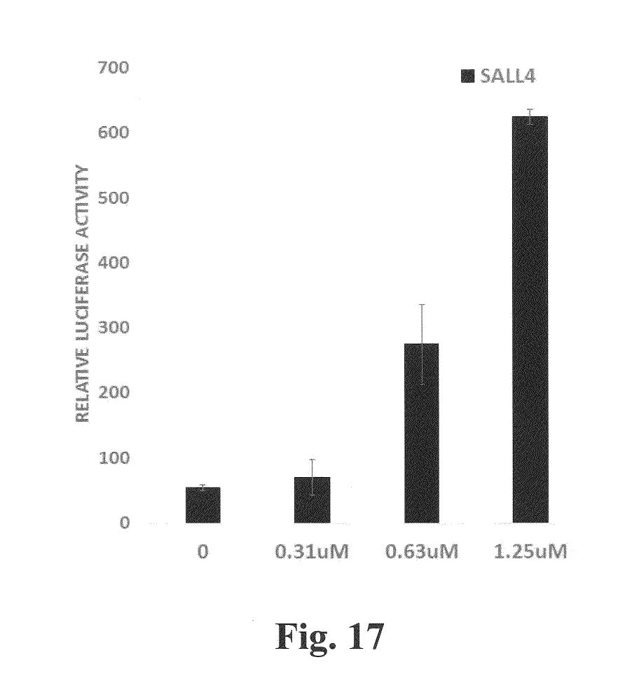

Previously we have shown that the OCT4 regulatory protein, SALL4 is a robust stimulator for ex vivo HSC/HPC expansion (Aguila et al, Blood, 2011, 118:576-85). We have screened small chemical compounds in order to replace SALL4 functions in the hematopoietic stem cell expansion. Our initial screen revealed that the TGF beta receptor-1 kinase inhibitor named 616452 was able to dramatically activate the SALL4 promoter (FIG. 17) and this activation was associated with enhancement of the HSC/HPC population on ex vivo cultures. CD34+ cells isolated from peripheral blood of G-CSF-mobilized donors were cultured for 5 days under minimal cytokines and the small molecule (10 μM), which we call SALL4 inducer. This compound rapidly expanded the CD34+ cells.

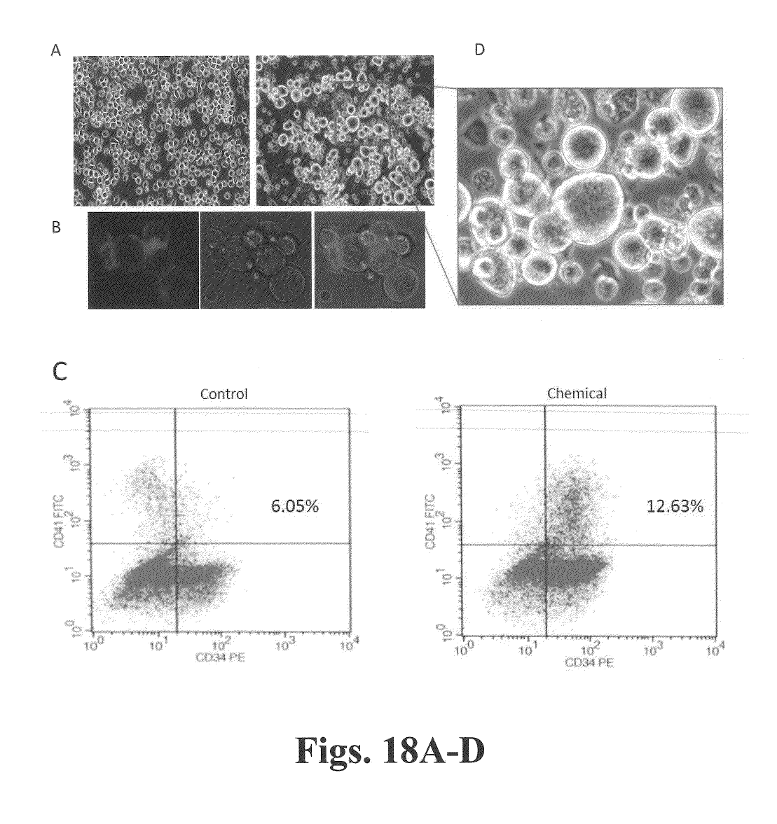

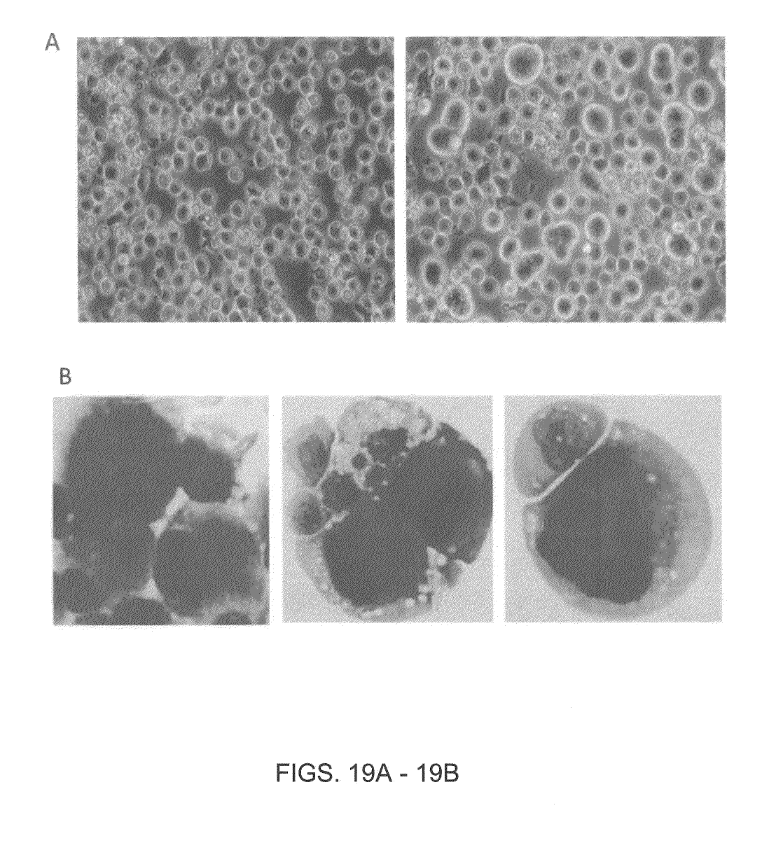

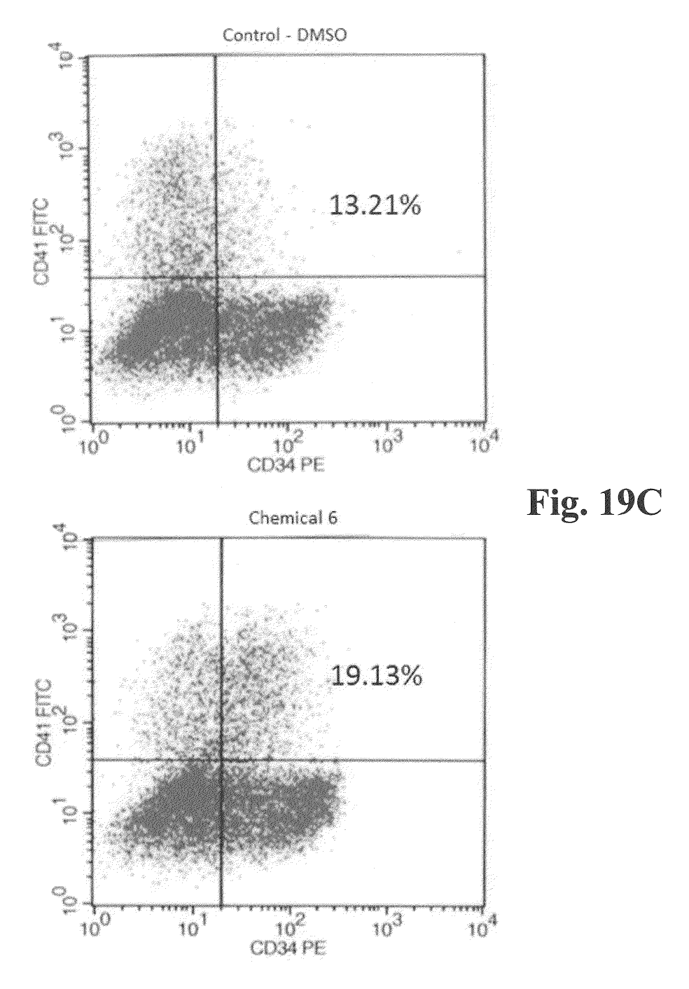

We also noticed a significant increase in large megakaryocyte-like cells when culturing CD34+ in the presence of the small molecule 616452 (10 μM) on day 5 compared to untreated cells (FIGS. 18-19).

By 10 days of culture, the large cells had taken over the culture (FIG. 18A). While still significant, the control still had the appearance of a few medium-large megakaryocytes that were around 4× the size of a typical CD34+ cells. Giemsa-Wright staining revealed these chemically treated cells to have a cell sizes as large as 90 μm with the average size being around 50 um (FIG. 19B). Live cell staining revealed all the large cells were positive for CD41, a marker unique to megakaryocytes throughout their development (FIG. 18B). Flow cytometry study was used to analyze the immunophenotype of cultured cells. As mature, large megakaryocytes may not be detectable by flow cytometry, bone marrow CD34+ cells were cultured for 4 days in order to detect younger, small megakaryocytes. The presence of 616452 in the culture yielded a 2-fold increase of CD34+CD41+ cells associated with the progenitor population of megakaryocytes (FIG. 18C). However, flow analysis of megakaryocytes is known to be misleading due to the sheer size of the megakaryocytes, which can have a diameter ranging from 20 um to 120 um. Analysis by flow cytometry revealed an increase of nearly 100% in the CD41+ population within 4 days of culture (FIG. 18C). Furthermore, the control cells had the loss of CD34 expression on the CD41+ cells while the chemically induced had a larger population of CD34+, indicating the capability of this chemical to also expand HSCs.

### Induction of Megakaryocyte Differentiation and Maturation in Cord Blood CD34+ Cells

Megakaryocytes derived from CB in vitro are usually smaller than megakaryocytes derived from bone marrow or mobilized peripheral blood from adults. Small megakaryocyte size may contribute to delayed platelet engraftment when cord blood is used for bone marrow transplantation. To test whether 616452 is able to increase fetal/neonatal megakaryocyte size and ploidy, CD34+ cells isolated from human umbilical CB were cultured in StemSpan SFEM containing SCF, TPO and Flt-3 Ligand. CB CD34+ cells in the presence of TPO after 4 or 8 day culture, were morphologically consistent of a relative homogenous population of small cells (FIG. 19A) and megakaryocytic features such as large nuclear size and cytoplasmic mass were hardly detectable. As compared to chemically induced CD34+ cells, megakaryocytes with these features were easily observed (FIGS. 19A and 19B). The finding is consistent with previous studies that TPO induction does not shorten megakaryocyte maturation time, while chemical small molecules could shorten this period.

The quantity of large cells and the size of the cells increased over time. By 8 days, the culture dishes were predominately composed of megakaryocytes and the formation of megakaryocytic clusters began. A Giemsa-Wright stain of these cells revealed a lobular multi-nucleated structure and a granular cytoplasmic nature characteristic of megakaryocytes (FIG. 19B). Flow analysis revealed an increase of 25-40% of the CD41⁺ population as well as an increase in the CD34⁺ CD41⁺ population (FIG. 19C) similar to that of bone marrow.

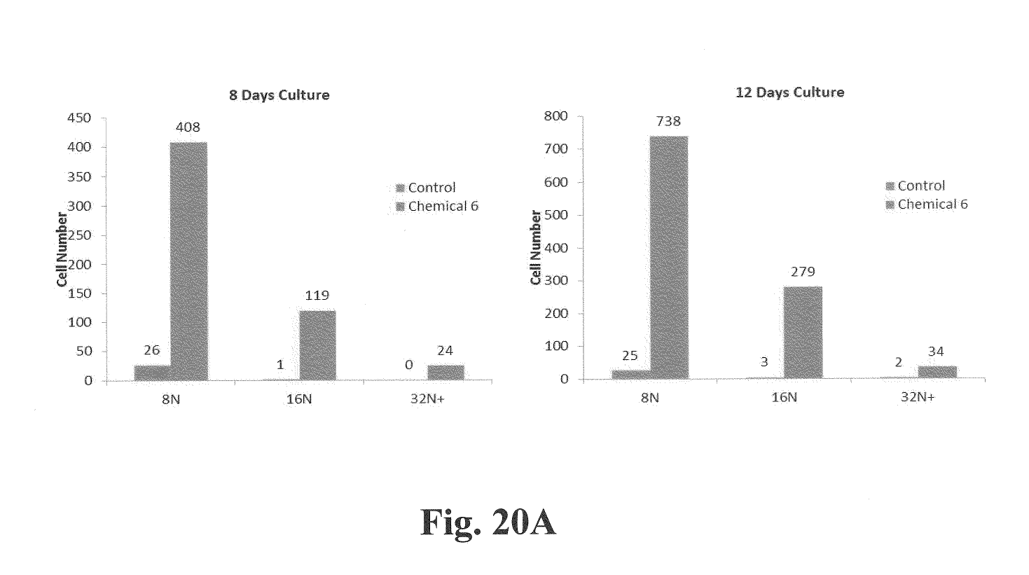

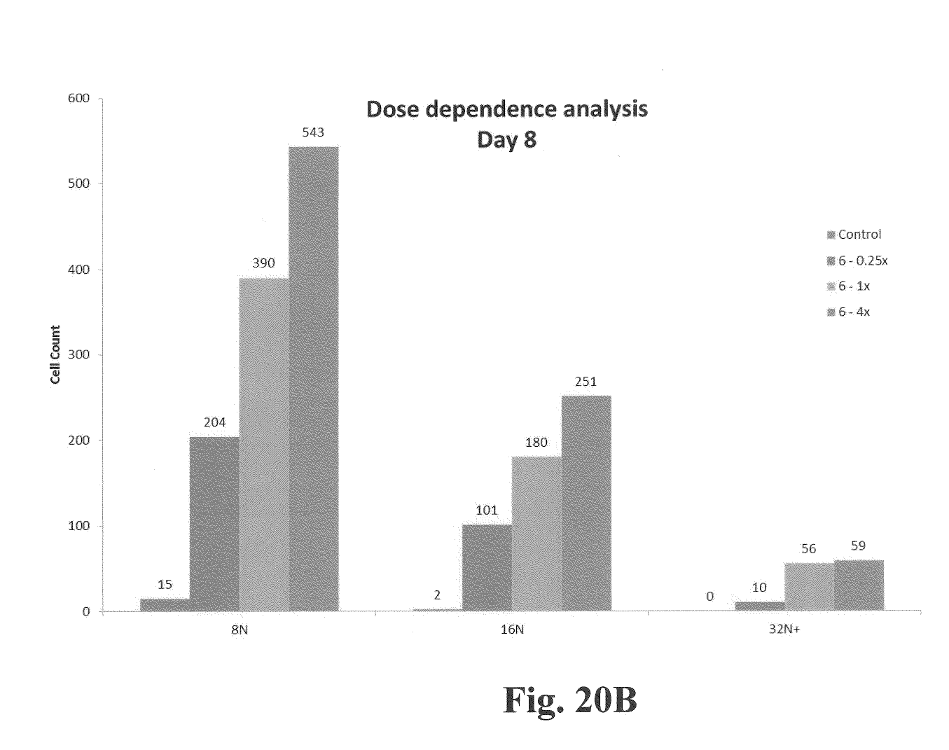

One of the unique characteristics of megakaryocytes is the ploidy development of their nuclei, which can be easily measured using propidium iodide (PI), a fluorescent intercalating agent that binds to nucleic acids. Megakaryocyte (Mk) ploidy correlates well with their maturation and platelet production. Cells induced with 616452 for 8 days revealed a drastically increased number of cells with greater ploidy. Out of 10,000 events, the control cells only elicited only 1 cell with a ploidy of 16N and nothing thereon greater (FIG. 20A). The chemically induced cells meanwhile produced over 100 cells of 16N ploidy and registered ploidy numbers as high as 64N. 616452 consistently induced greater number of cells in each ploidy category 4N and above.

A time dependence study of the chemical induction revealed that the longer the cells were induced, the greater the ploidy development. Between 8 and 12 days of induction, the number of 8N, 16N and 32N cells nearly doubled (FIG. 20A), while the control remains almost unchanged. However, if the chemical is removed at certain time points and cultured in the presence of only TPO, as the control is, the ploidy development is essentially halted. For every 24 hours the cells were induced, the ploidy numbers doubled, up until after 4 days of culture. This is likely due to the aggregation of the megakaryocytes into clusters. This aggregation begins with small clusters by day 6 and significantly larger clusters by day 8 of induction. The aggregation impedes on the accuracy of the flow results. Once clustered, the cells become hard to dissociate without the accidental lysis of the larger megakaryocytes, greatly affecting the analysis of the larger cells of 32N or higher after day 6. Regardless, the difference of megakaryocyte maturation is drastic with fold increases of 5 for 4N cells, 30 for 8N and 80 for 16N.

The next question was to address whether the dose affected the maturation rate. The small chemical had noticeable inhibition on cell growth and at concentrations of 100 μM, all the cells died within 48 hours. The previously published concentration of 10 μM was used as the reference standard of 1× where the growth of the cells was slowed to 25-50% of the control. Additional concentrations were tested (FIG. 20B) and as confirmed in multiple experiments with different patients, there was essentially little to no ploidy development past 16N in the control. In nearly all the categories, the greater the dose, the greater the ploidy development with a 1.68 fold increase over the standard in 8N cells, 2.5 fold in 16N and 6 fold in 32N. It was also observed that at the higher ploidies, there was a lack of cluster formation, with no clusters in 4× concentrations or higher.

### Extent of CD41+ Megakaryocytic Population in Response to the Induction of 616452

We tested 616452 on CD34⁺ cells isolated from hUCB, BM and mobilized PB. While the chemically induced samples increased the presence and ploidy of megakaryocytes in all samples, the quantity differed drastically between patients. While the time frame was the same for the appearance of the changes, some patients produced a high number of large cells after a week in culture. All patients were healthy donors free of any disease, leading us to question the possibility that this chemical has specificity for megakaryocytic cells which can differ vastly dependent on the number of megakaryocytic stem/progenitor cells present in the samples.

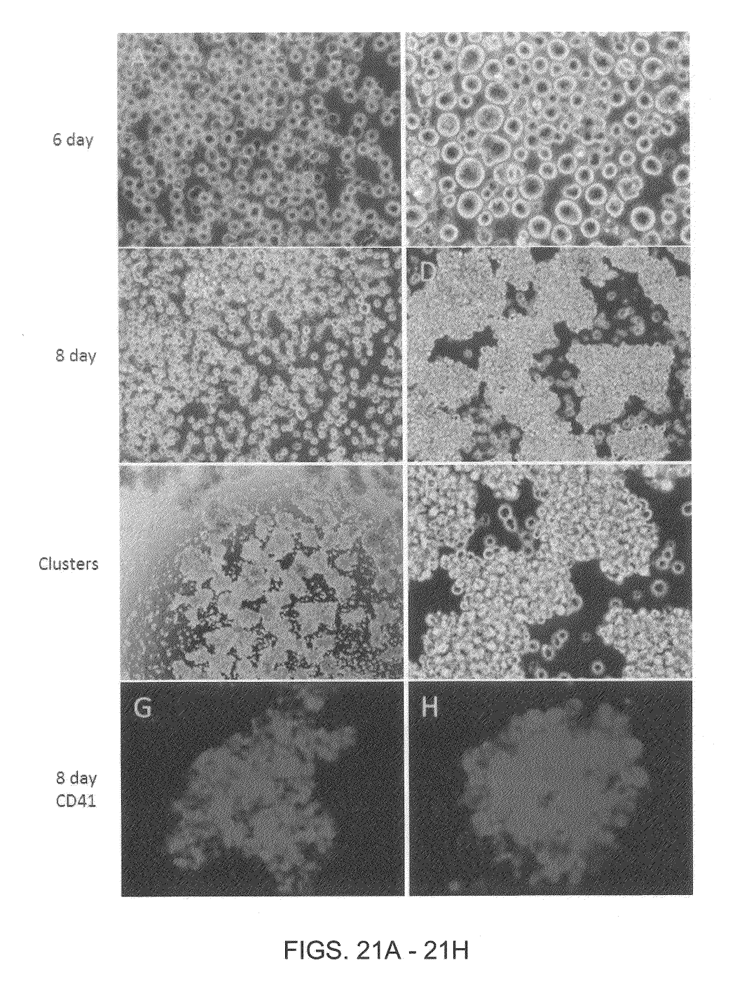

To test this issue and the extent of megakaryocytic population in response to the chemical induction, we cultured HUBC CD34⁺ cells for 12-14 days in a media specific to megakaryocyte expansion (Nikougoftar Zarif, M., et al., Cell journal 13:173-178 (2011)). The cells were labeled with a conjugated CD41 antibody and sorted by flow cytometry sorting. The resulting CD41⁺cells were induced with the small chemical and cells began to increase their size within 2 days. By day 6, approximately 80% of the cells appeared to be large in size by eye while the control had little to none. Aggregation of the cells began around day 4 with clusters of about 5-15 cells, 25-50 cells by day 6 and by day 8 the majority of cells were in cluster of hundreds of cells. The control meanwhile had little clusters, but none that compared in size and numbers as the induced cells (FIGS. 21C-F).

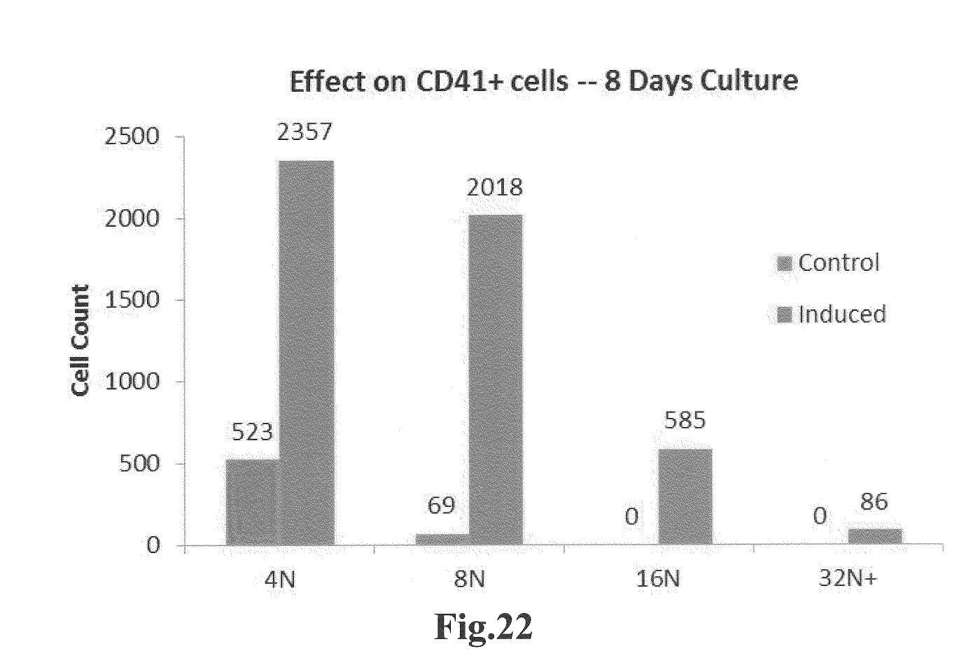

A live cell stain revealed that 95% of the cells that were present after 8 days of induction with the small chemical were positive. The live cell stain also gave some insight into the structure of the large cell clusters that were only becoming larger and larger (FIGS. 21 G-H). The clusters appeared to be specifically attracting only the large cells. The majority of the cells inside the clusters appeared to be at least 4× the size of a typical hematopoietic stem cell. Furthermore a ploidy analysis of the cells after 8 days of induction revealed that approximately 50% of the cells had a ploidy number of 4N or greater while the control cells induced with only TPO had only 6% of the cells with a ploidy of 4N or greater. Out of 10,000 events, there were 69 cells with 8N ploidy and nothing greater in the control cells. Meanwhile, in our small chemical induced cells, we observed 2018 cells of 8N, 585 cells of 16N and 86 cells of 32N or greater (FIG. 22). CD41− cells were also cultured under the same conditions with the control being induced using TPO and DMSO. There was little to no ploidy development in the control and a slight increase in ploidy of the 616452 induced cells.

### Synergistic Effect of Different TGF-Beta Inhibitors on CB Megakaryocyte Maturation

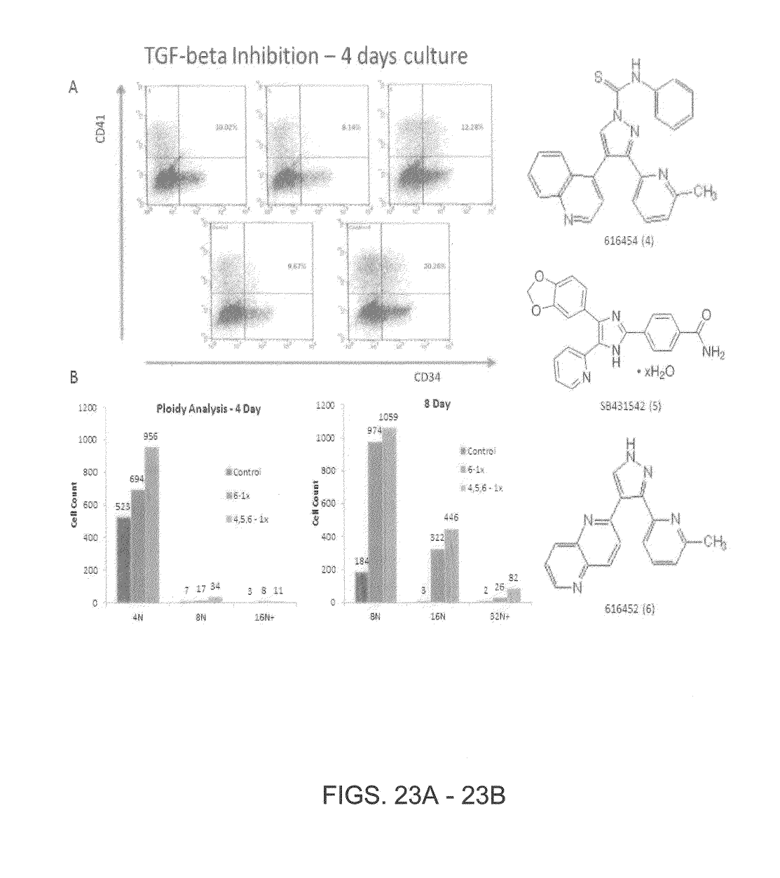

We next investigated some other TGF-beta receptor I inhibitors, 616454, SB431542 in the induction of megakaryocyte maturation. When induced with 616454, the cells experienced no increase in ploidy number and flow analysis of CD41 revealed no any significant difference from control after 4 days of culture (FIGS. 23A and 23B). Meanwhile SB431542 revealed a marginal decrease in the CD41 population with also no significant deviation in ploidy development. However in combination with 616452, these three chemicals exhibited a synergistic effect that significantly enhanced both the enrichment of the CD41 population and the maturation of the megakaryocytes. Compared to control, the combined chemical cocktail increased CD41 expression by 109% compared to the 27% by 616452 alone. Ploidy development exhibited greater ploidy numbers in every category compared to 616452 alone (FIG. 23B). An additional TGF-beta receptor kinase inhibitor, LY364947 was also tested and there was no significant increase in the number of large megakaryocytes compared to that of control when this compound was added to the CB CD34+ differentiation medium for 4 day culture.

### Gene Expression Profile to Study the Action of 616452

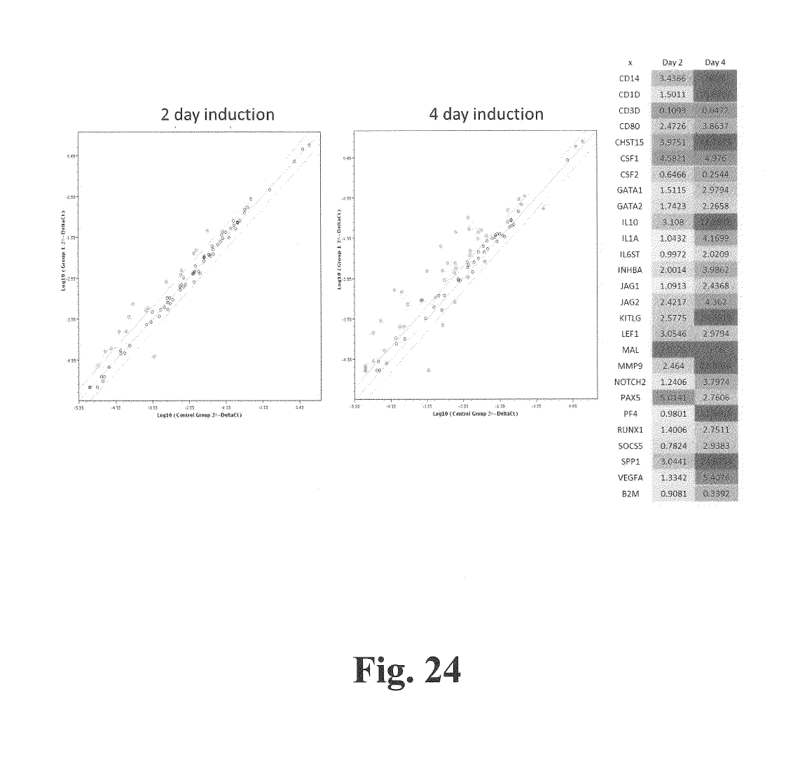

Cells were analyzed via Qiagen's PAHS-054Z hematopoiesis array containing 81 genes. Since maturation does not visually occur until day 4 into induction and genes activated by chemicals are typically expressed within hours, cells induced for 2 and 4 days were compared to un-induced controls. There is a significant difference between 2 and 4 day induced cells with about fifteen genes up-regulated and one down-regulated on day 2 and thirty eight genes up-regulated and four genes down-regulated as of day 4 (FIG. 24). Most noticeable is the up-regulation of genes that include GATA1, GATA2, JAG1, JAG2, PF4, RUNX1, CD14, CD1D, CD3D, CD80, CHST15, CSF1, CSF2, IL10, 1L1A, IL6ST, INHBA, KITLG, LEF1, PF4, SOCS5, SPP1, VEGFA, and NOTCH2. In many of these genes, the expression is nearly doubled between day 2 and day 4. Furthermore, the expression of PF4, platelet factor 4, a chemokine released from activated platelets during platelet aggregation was found to be identical compared to control on day 2, when there were no visual indicators of megakaryocyte maturation. However, on day 4 alongside the appearance of large megakaryocytes, the small cytokine was found to be expressed more than 11-fold compared to that on day 2.

### Robust Enhancement of Bone Marrow Recovery by 616452 In Vivo.

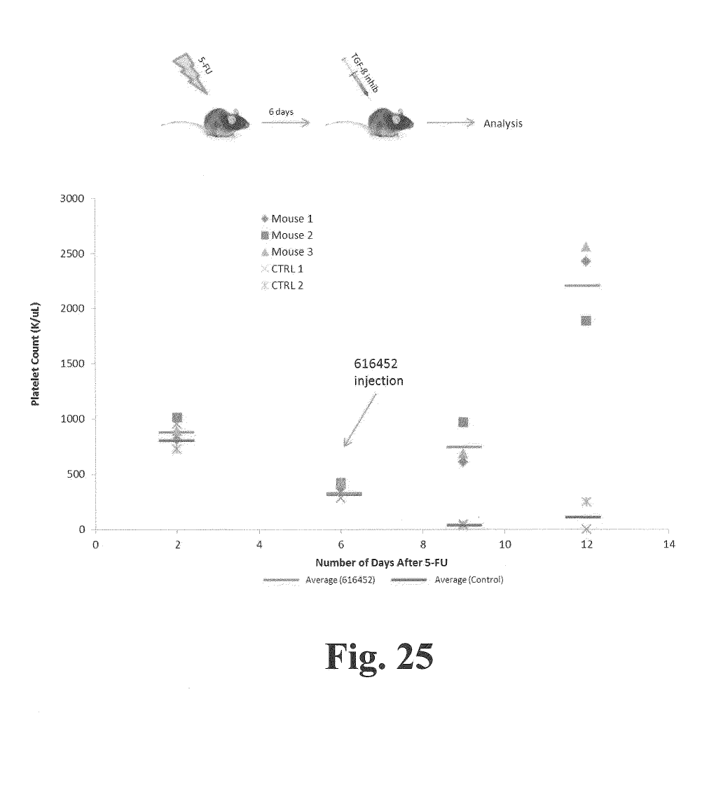

Fifteen 9.5 week old C57/B6 male mice were given chemotherapy and analyzed for platelet and white counts over a period of 2 weeks in the presence and absence of 616452 (10 mg/kg) (FIG. 25). The complete blood counts (CBCs) all looked normal without any change within the first 4 days. By day 6, the CBCs began to show changes to the mice as some mice became lethargic. 616452 was introduced to eight of the mice at this point and was injected every other day for a total of three doses. The next analysis revealed that many of the mice showed increases in platelet counts while the control mice, injected with DMSO/PBS solution, continued to decrease in platelet counts. By day 12, the control mice began to recover, while the chemical-induced mice had platelet counts through the roof, with almost twice as many platelets as normal mice. The mice that were not given 5-FU, but were injected on the chemical showed little to no change with platelet counts alternating around the normal range.

### Directly Dedifferentiating or Reprogramming of AP Cells to Multiple Stem Cells or Immature Cells

We adopted a hypothesis driven approach by screening small molecules that target cellular mechanisms that are known to influence ESC pluripotency and OCT4 functions. We narrowed down a combination of eight chemical compounds (8-chemical) or six chemical compounds (6-chemical) that directly reprogram AF cells to multipotent stem cells or immature cells. We found that 6-Chemical was toxic to AF cells at 1× and 0.5× concentrations, 8-chemical was toxic at 1×, 0.5× and 0.25× concentrations. While AF cells could keep proliferating in 0.25×, for 6-chemical and 0.125× for 8-chemical. AF cells were seeded at a density of 10,000/well in a 24-well tissue culture plate and allowed to grow overnight in MSC medium before chemical treatment.

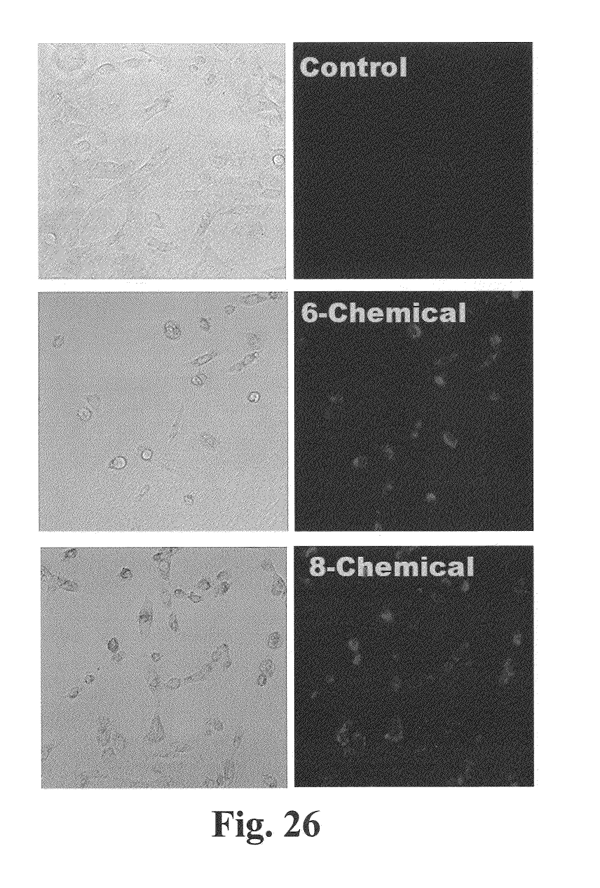

On day 4, the non-toxic groups including in 0.25× 6-chemical and 0.125× 8-chemical wells were replated at a 1:4 ratio and maintained under same conditions. Cells in both groups kept proliferating though relatively slower than those of the control group. On day 7, almost all of the cells became positive for FLK1 expression in both 6-chemical and 8-chemical groups, as compared the negative staining in cells of control group (FIG. 26). Of note is that there was no obvious difference regarding to FLK1 expression between 6-chemical and 8-chemical group.

### FLK1 Expression is Dependent on the Presence of Chemicals

The FLK1 expressed cells were re-plated after they reached confluence and cultured in EGM2 media with or without the chemicals. Four days later, FLK1 expression was decreased in the culture without chemicals in both 6-chemical and 8-chemical groups as compared to the maintained FLK1 expression of cells under the presence of chemicals.

FLK1+ Cells Induced by Chemicals Differentiate into Endothelial Cells

Ten days after 6-chemical or 8-chemical treatment, the FLK1+ cells were allowed to differentiate after removal of the chemicals. As early as 3 days in differentiation media (EGM2), CD31 positive cells could be detected in cells from 8-chemical group, while no cells were CD31 positive in the 6-chemical group at this point. Until day 7 after differentiation, CD31 positive cells were observed in both 6-chemical and 8-chemical groups, with the intensity and percentage of CD31+ cells higher in the 8-chemical group. Another specific marker, VE-cadherin was seen to express in cells from both 6- and 8-chemical groups. Ac-LDL uptake assay was also used to evaluate the functional feature of the differentiated cells. Consistent with endothelial functions, cells in both two chemical groups were able to take up ac-LDL as compared to the inability of control cells.

What is claimed is:

1. A method for expanding a stem cell or immature cell, comprising contacting said cell with one or more compounds selected from: a cyclic AMP (cAMP) agonist, a lysine-specific demethylase 1 (LSD1) inhibitor, a transforming growth factor-beta receptor (TGF-βR) inhibitor, a lysine methyltransferase EZH2 (KMT6) inhibitor, a GLP histone lysine methyltransferase inhibitor, a G9a histone lysine methyltransferase inhibitor, and a histone methyltransferase (HMTase) G91 inhibitor.

2. The method of claim 1, wherein the stem cell or immature cell is a stem cell or immature endothelial cell, amniotic fluid cell, bone marrow cell, or a stem cell or immature cell of the brain, liver, skin, heart, kidney, pancreas, gall bladder, intestine, skeletal muscle, or lung.

3. The method of claim 1, wherein the stem cell is a hematopoietic stem cell.

4. The method of claim 3, wherein the hematopoietic stem cell is a hematopoietic stem cell of the bone marrow, umbilical cord blood, peripheral blood, placenta, or spleen.

5. The method of claim 1, where the LSD1 inhibitor is selected from 2-(1R,2S)-2-(4-(Benzyloxy)phenyl)cyclopropylamino)-1-(4-methylpiperazin-1-yl)ethanone, tranylcypromine hydrochloride, and functional derivatives thereof.

6. The method of claim 1, wherein the TGF-βR inhibitor is selected from 2-[3-(6-methyl-2-pyridinyl)-1H-pyrazol-4-yl]-1,5-naphthyridine, and functional derivatives thereof.

7. The method of claim 1, wherein the EZH2 inhibitor is selected from 5R-(4-amino-1H-imidazo[4,5-c]pyridin-1-yl)-3-(hydroxymethyl)-3-cyclopentene-1 S,2R-diol, and functional derivatives thereof.

8. The method of claim 1, wherein the HMTase G91 inhibitor is selected from 2-(Hexahydro-4-methyl-1H-1,4-diazepin-1-yl)-6,7-dimethoxy-N-[1-(phenylmethyl)-4-piperidinyl]-4-quinazolinamine trihydrochloride hydrate, and functional derivatives thereof.

9. The method of claim 1, wherein the inhibitor of G9a or GLP histone lysine methytransferase is selected from 2-cyclohexyl-6-methoxy-N-[1-(1-methylethyl)-4-piperidinyl]-7-[3-(1-pyrrolidinyl)propoxy]-4-quinazolinamine, and functional derivatives thereof.

10. The method of claim 1, wherein the cell population is cultured in media comprising Iscove's modified Dulbecco's medium with bovine serum albumin, human insulin, human transferrin, 2-mercaptoethanol, and supplemented with one or more of fetal bovine serum, thrombopoietin, Flt-3 ligand, stem cell factor, interleukin-3 (IL-3), interleukin-6 (IL-6), interleukin-9 (IL-9), granulocyte colony-stimulating factor, and nerve growth factor.

11. The method of claim 1, wherein a population of stem cells or immature cells is expanded 10-fold.

12. The method of claim 1, wherein said stem cell or immature cell is expanded in vivo.

13. The method of claim 1, wherein said stem cell or immature cell is expanded ex vivo or in vitro and administered to a subject.

14. The method of claim 13, wherein the stem cell or immature cell is autologous to said subject.

15. A kit for carrying out a method according to claim 1.

16. An ex vivo expanded cell preparation obtained by the method of claim 1.

17. A method according to claim 13, wherein the subject has a condition or disease treatable by administration of expanded hematopoietic cells.

18. A therapeutic method comprising administration of one or more compounds selected from a cAMP agonist, a lysine-specific demethylase 1 (LSD1) inhibitor, a transforming growth factor-beta receptor (TGF-βR) inhibitor, a lysine methyltransferase EZH2 inhibitor, a GLP histone lysine methyltransferase inhibitor, a G9a histone lysine methyltransferase inhibitor, and a histone methyltransferase (HMTase) G91 inhibitor, to a subject.

19. A method to generate a multipotent or immature cell from a mature somatic cell, comprising contacting said mature somatic cell with one or more compounds selected from: an HDAC inhibitor; a transforming growth factor-beta receptor (TGF-βR) inhibitor II; an ALK4, ALK5 and ALK7 inhibitor; a glycogen synthase kinase 3 (GSK3) inhibitor; a lysine methyltransferase EZH2 inhibitor; a histone-lysine methyltransferase (HMTase) inhibitor; an inhibitor of the histone lysine demethylase LSD1; and a histone methyltransferase G9a/GLP inhibitor.

20. The method of claim 19, wherein the mature somatic cell is selected from an umbilical cord blood cell, an amniotic fluid cell, a bone marrow cell, a blood cell, a myocardial cell, a dermal or epidermal cell, a pancreatic cell, an endothelial cell or a fibroblast.

21. The method of claim 19, wherein:
a. said HDAC inhibitor is valproic acid, or a functional derivative thereof;
b. said TGF-βR inhibitor II is 2-[3-(6-methyl-2-pyridinyl)-1H-pyrazol-4-yl]-1,5-naphthyridine, or a functional derivative thereof;
c. said ALK4, ALK5 and ALK7 inhibitor is 3-(6-Methyl-2-pyridinyl)-N-phenyl-4-(4-quinolinyl)-1H-pyrazole-1-carbothioamide, or a functional derivative thereof;
d. said GSK3 inhibitor is 6-[[2-[[4-(2,4-dichlorophenyl)-5-(5-methyl-1H-imidazol-2-yl)-2 pyrimidinyl]amino]ethyl]amino]-3-pyridinecarbonitrile, or a functional derivative thereof;
e. said lysine methyltransferase EZH2 inhibitor is 5R-(4-amino-1H-imidazo[4,5-c]pyridin-1-yl)-3-(hydroxymethyl)-3-cyclopentene-1S,2R-diol, or a functional derivative thereof;
f. said HMTase inhibitor is 2-(Hexahydro-4-methyl-1H-1,4-diazepin-1-yl)-6,7-dimethoxy-N-[1-(phenylmethyl)-4-piperidinyl]-4-quinazolinamine trihydrochloride hydrate, or a functional derivative thereof;
g. said LSD1 inhibitor is tranylcypromine hydrochloride, or a functional derivative thereof; and/or
h. said histone methyltransferase G9a/GLP inhibitor is 2-Cyclohexyl-6-methoxy-N-[1-(1-methylethyl)-4-piperidinyl]-7-[3-(1-pyrrolidinyl)propoxy]-4-quinazolinamine, or a functional derivative thereof.

22. The method of claim 19, wherein the generated cells are cultured in media comprising DF12, 15% FBS and 10 ng/ml bFGF.

23. The method of claim 19, wherein the generated multipotent or immature cell is an ALK+ cell.

24. The method of claim 23, wherein the generated ALK+ cell can be differentiated into a cell of the brain, liver, skin, heart, kidney, pancreas, gall bladder, intestine, skeletal muscle or lung.

25. The method of claim 24, wherein the generated ALK+ cell is differentiated into an endothelial cell by culturing said ALK+ cell in endothelial growth medium.

26. An ALK+ cell produced by the method of claim 19.

27. An endothelial cell produced by the method of claim 25.

28. The endothelial cell of claim 27, wherein said endothelial cell expresses CD31 and/or VE-cadherin.

29. The endothelial cell of claim 27, wherein said endothelial cell is capable of uptake of acetylated-low density lipoprotein (Ac-LDL).

30. The endothelial cell of claim 27, wherein said endothelial cell is autologous to a post-natal individual.

31. A method for treating a genetic disorder or regenerating an organ or tissue, comprising administering the endothelial cell of claim 27 to a subject in need thereof.

32. A method of generating an insulin-producing pancreatic beta cell from a pancreatic islet cell, comprising expressing OCT4 or an OCT4 functional homolog or derivative, under the control of a high expressing promoter, in said pancreatic islet cell.

33. The method of claim 32, wherein the high expressing promoter is the spleen focus forming virus (SFFV) promoter or the human elongation factor 1α (EF) promoter.

34. An insulin-producing pancreatic beta cell produced by the method of claim 32.


SEQUENCE LISTING (54 sequences)

SEQ ID NO: 1 - DNA, 423 bp, Spleen focus-forming virus
agctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggtacc

SEQ ID NO: 2 - DNA, 1178 bp, Homo sapiens
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 3 - DNA, 18 bp, Artificial Sequence
  sense primer
aagacttccctcagcttt

SEQ ID NO: 4 - DNA, 18 bp, Artificial Sequence
  anti-sense primer
ggagcaaagatccaagac

SEQ ID NO: 5 - DNA, 18 bp, Artificial Sequence
  sense primer
cgagtggaaacttttgtc

SEQ ID NO: 6 - DNA, 18 bp, Artificial Sequence
  anti-sense primer
cagcgtgtacttatcctt

SEQ ID NO: 7 - DNA, 18 bp, Artificial Sequence
  sense primer
gactcgaacgaagaagat

SEQ ID NO: 8 - DNA, 18 bp, Artificial Sequence
  anti-sense primer
atgtcctcactctcaaac

SEQ ID NO: 9 - DNA, 18 bp, Artificial Sequence
  sense primer
gtagttgttaccgctctt

SEQ ID NO: 10 - DNA, 22 bp, Artificial Sequence
  anti-sense primer
gaaatgctcagatacataaagt

SEQ ID NO: 11 - DNA, 20 bp, Artificial Sequence
  sense primer
tgaagcaagaatacaggtat

SEQ ID NO: 12 - DNA, 18 bp, Artificial Sequence
  anti-sense primer
ggaattggttggtagaca

SEQ ID NO: 13 - DNA, 19 bp, Artificial Sequence
  sense primer
tcccactaaaccctataca

SEQ ID NO: 14 - DNA, 18 bp, Artificial Sequence
  anti-sense primer
ctctgatgcctgaacatt

SEQ ID NO: 15 - DNA, 18 bp, Artificial Sequence
  sense primer
atgtgatgctcaatggat

SEQ ID NO: 16 - DNA, 19 bp, Artificial Sequence
  anti-sense primer
agtctctggaatcttctct

SEQ ID NO: 17 - DNA, 19 bp, Artificial Sequence
  sense primer
gcttaaactcttggcattt

SEQ ID NO: 18 - DNA, 18 bp, Artificial Sequence
  anti-sense primer
tttgaggtttggtgactt

SEQ ID NO: 19 - DNA, 19 bp, Artificial Sequence
  sense primer
ggttctatttgggaaggta

SEQ ID NO: 20 - DNA, 18 bp, Artificial Sequence
  anti-sense primer
atactggttcgctttctc

SEQ ID NO: 21 - DNA, 20 bp, Artificial Sequence
  sense primer
ggcaagggaaagacgagtca

SEQ ID NO: 22 - DNA, 20 bp, Artificial Sequence
  anti-sense primer
gggtcttcctccttgtgagc

SEQ ID NO: 23 - DNA, 20 bp, Artificial Sequence
  sense primer
acccagtgcaaggaaaacca

SEQ ID NO: 24 - DNA, 20 bp, Artificial Sequence
  anti-sense primer
acctggcaaactgggtgaaa

SEQ ID NO: 25 - DNA, 20 bp, Artificial Sequence
  sense primer
gacccgctgagacattaccc

SEQ ID NO: 26 - DNA, 20 bp, Artificial Sequence
  anti-sense primer
tgttgcacgaattcatccgc

SEQ ID NO: 27 - DNA, 20 bp, Artificial Sequence
  sense primer
cgccaggattcacctttcct

SEQ ID NO: 28 - DNA, 20 bp, Artificial Sequence
  anti-sense primer
ttcctcggaaaaagggaccg

SEQ ID NO: 29 - DNA, 20 bp, Artificial Sequence
  sense primer
ggcaagggcaactactggat

SEQ ID NO: 30 - DNA, 20 bp, Artificial Sequence
  anti-sense primer
ctgagtcaacacggagctgt

SEQ ID NO: 31 - DNA, 20 bp, Artificial Sequence
  sense primer
tgtgactggtcgtgacatgg

SEQ ID NO: 32 - DNA, 20 bp, Artificial Sequence
  anti-sense primer
ctagtggcggtcataggcag

SEQ ID NO: 33 - DNA, 20 bp, Artificial Sequence
  sense primer
acagcagccggaacctaaaa

SEQ ID NO: 34 - DNA, 20 bp, Artificial Sequence
  anti-sense primer
cctttttgcagccgaagtcc

SEQ ID NO: 35 - DNA, 20 bp, Artificial Sequence
  sense primer
tctccttcctggtcctagcc

SEQ ID NO: 36 - DNA, 20 bp, Artificial Sequence
  anit-sense primer
tgcaccagggaggcaattag

SEQ ID NO: 37 - DNA, 20 bp, Artificial Sequence
  sense primer
cgctcagtggttgtagcaga

SEQ ID NO: 38 - DNA, 20 bp, Artificial Sequence
  anti-sense primer
agctgttgcataagccacct

SEQ ID NO: 39 - DNA, 20 bp, Artificial Sequence
  sense primer
gcagtttggtcattgggctg

SEQ ID NO: 40 - DNA, 20 bp, Artificial Sequence
  anti-sense primer
tttggcatcgatgaagggct

SEQ ID NO: 41 - DNA, 20 bp, Artificial Sequence
  sense primer
ctggccatccagagcaagaa

SEQ ID NO: 42 - DNA, 20 bp, Artificial Sequence
  anti-sense primer
cgtacatctcgccctcttcc

SEQ ID NO: 43 - DNA, 20 bp, Artificial Sequence
  sense primer
cagatccagtacgcgcagat

SEQ ID NO: 44 - DNA, 20 bp, Artificial Sequence
  anti-sense primer
cggcatgcttcgatttccag

SEQ ID NO: 45 - DNA, 20 bp, Artificial Sequence
  sense primer
gcacactcacatccacctga

SEQ ID NO: 46 - DNA, 20 bp, Artificial Sequence
  anti-sense primer
ccttgcagacacctcctctg

SEQ ID NO: 47 - DNA, 20 bp, Artificial Sequence
  sense primer
tgcagcctagtaggagctga

SEQ ID NO: 48 - DNA, 20 bp, Artificial Sequence
  anti-sense primer
cctccgggttagggtctaca

SEQ ID NO: 49 - DNA, 20 bp, Artificial Sequence
  sense primer
ggatcacccatggctagacg

SEQ ID NO: 50 - DNA, 20 bp, Artificial Sequence
  anti-sense primer
tctgtctctgcagctgtgtg

SEQ ID NO: 51 - DNA, 20 bp, Artificial Sequence
  sense primer
ctctgagaccctccgcaaag

SEQ ID NO: 52 - DNA, 20 bp, Artificial Sequence
  anti-sense primer
ctaagaggtcaaggcccgtc

SEQ ID NO: 53 - DNA, 18 bp, Artificial Sequence
  sense primer
caccacaccttctacaat

SEQ ID NO: 54 - DNA, 18 bp, Artificial Sequence
  anti-sense primer
tgatctgggtcatcttct